(19) United States
(12) Patent Application Publication
(10) Pub. No.: US 2021/0177951 A1
(43) Pub. Date: Jun. 17, 2021

(54) ENGINEERED CELLS & METHODS

(71) Applicant: Novoscope IP Limited, London (GB)

(72) Inventor: Jasper CLUBE, London (GB)

(73) Assignee: Novoscope IP Limited, London (GB)

(21) Appl. No.: 17/179,815

(22) Filed: Feb. 19, 2021

Related U.S. Application Data
(62) Division of application No. 16/076,479, filed on Aug. 8, 2018, now abandoned.

(30) Foreign Application Priority Data
Feb. 19, 2016 (GB) 1602974.6

(51) Int. Cl. A61K 39/00; C07K 14/725; C07K 16/28; A61K 39/395; C07K 14/705

(52) U.S. Cl. CPC A61K 39/0011; C07K 14/7051; C07K 16/2803; C07K 16/2809; A61K 39/3955; A61K 39/39558; C07K 2319/02; C07K 14/70578; C07K 2319/03; C07K 2317/31; A61K 2039/505; A61K 2039/5156; C07K 14/70521

(57) ABSTRACT

The present invention relates to engineered cells, engineered chimaeric antigen ligands (CALs), and novel uses of multi-specific binding agents for bridging cells. The invention also provides variation-matched engineered cells.

### CROSS-REFERENCE TO RELATED APPLICATIONS

This application is divisional under 35 U.S.C. § 121 of co-pending U.S. application Ser. No. 16/076,479 filed Aug. 8, 2018, which is a 35 U.S.C. § 371 National Phase Entry Application of International Application No. PCT/EP2017/053185 filed Feb. 13, 2017, which designates the U.S. and claims benefit under 35 U.S.C. § 119(a) of GB Provisional Application No. 1602974.6 filed Feb. 19, 2016, the contents of which are incorporated herein by reference in their entireties.

### FIELD OF THE INVENTION

The present invention relates to novel uses of multi-specific binding agents for bridging engineered immune cells (eg, CAR- or CAL T-cells) to target cells, engineered immune cells, engineered chimaeric antigen ligands (CALs) and methods of immunotherapy, eg, adoptive CAR- or CAL T-cell therapy of humans. The invention also provides human variation-matched CAR- and CAL-cells for carrying out Precision Immunotherapy.

### SEQUENCE LISTING

The instant application contains a Sequence Listing which has been submitted electronically in ASCII format and is hereby incorporated by reference in its entirety. Said ASCII copy, created on Aug. 8, 2018, is named 2018-08-08-SEQUENCE-LISTING-083720-093140USPX.txt and is 27,414 bytes in size.

### BACKGROUND

One approach to immunotherapy involves engineering patients' own (or a donor's) immune cells to express cell-surface antigen receptors (CARs) that recognise and attack tumours. Although this approach, called adoptive cell transfer (ACT), has been restricted to small clinical trials so far, treatments using these engineered immune cells have generated some remarkable responses in patients with advanced cancer.

The Chimeric Antigen Receptor (CAR) consists of an antibody-derived targeting domain fused with T-cell signaling domains that, when expressed by a T-cell, endows the T-cell with antigen specificity determined by the targeting domain of the CAR. CARs can potentially redirect the effector functions of a T-cell towards any protein and non-protein target expressed on the cell surface as long as an antibody-based targeting domain is available. This strategy thereby avoids the requirement of antigen processing and presentation by the target cell and is applicable to non-classical T-cell targets like carbohydrates. This circumvention of HLA-restriction means that the CAR T-cell approach can be used as a generic tool broadening the potential of applicability of adoptive T-cell therapy. See, eg, Methods Mol Biol. 2012; 907:645-66. doi: 10.1007/978-1-61779-974-7_36, “Chimeric antigen receptors for T-cell based therapy”, Cheadle E J et al;

The first CAR-T construct was described in a 1989 paper by immunotherapy pioneer Zelig Eshhar in PNAS. The structure of the CAR now comprises a transmembrane polypeptide chain which is a chimaera of different domains from different cellular proteins. For example, the CAR has an extracellular part joined (often by a linker and/or a hinge region) to an intracellular part, with a transmembrane portion of the CAR embedding the receptor in the membrane of an immune cell, normally a T-cell. The extracellular moiety includes an antibody binding site (usually in the form of an scFv, such as derived from a mouse mAb) that recognizes a target antigen, that commonly is a tumour associated antigen (TAA) on the surface of cancer cells. Antigen recognition in this way dispenses with the need to rely on TCRs that require MHC-restricted antigen presentation, and where binding affinities may be relatively low. The intracellular moiety of the CAR typically includes a CD3-zeta (CD3ζ) domain for intracellular signaling when antigen is bound to the extracellular binding site. Later generation CARs also include a further domain that enhances T-cell mediated responses, which often is a 4-1BB (CD137) or CD28 intracellular domain. On encountering the cognate antigen ligand for the CAR binding site, the CAR can activate intracellular signaling and thus activation of the CAR T-cell to enhance tumour cell killing.

Most CAR-Ts expand in vivo so dose titration in a conventional sense is difficult, and in many cases the engineered T-cells appear to be active “forever”—i.e., the observation of ongoing B-cell aplasia seen in most of the CD19 CAR-T clinical studies to date. This poses a serious problem for CAR T-cell approaches. Some observed risks are discussed in Discov Med. 2014 November; 18(100):265-71, “Challenges to chimeric antigen receptor (CAR)-T cell therapy for cancer”, Magee M S & Snook A E, which explains that the first serious adverse event following CAR-T cell treatment occurred in a patient with colorectal cancer metastatic to the lung and liver (Morgan et al., 2010). This patient was treated with T cells expressing a third-generation CAR targeting epidermal growth factor receptor 2 (ERBB2, HER2). The CAR contained an scFv derived from the 4D5 antibody (trastuzumab) that is FDA approved for the treatment of HER2-positive breast cancers (Zhao et al., 2009). The patient developed respiratory distress within 15 minutes of receiving a single dose of 10¹⁰ CAR-T cells, followed by multiple cardiac arrests over the course of 5 days, eventually leading to death. Serum analysis four hours after treatment revealed marked increases in the cytokines IFNγ, GM-CSF, TNFα, IL-6, and IL-10. CAR-T cells were found in the lung and abdominal and mediastinal lymph nodes, but not in tumour metastases. The investigators attributed toxicity to recognition of HER2 in lung epithelium resulting in inflammatory cytokine release producing pulmonary toxicity and cytokine release syndrome (CRS) causing multi-organ failure (Morgan et al., 2010). Trials utilizing second-generation HER2-targeted CARs derived from a different antibody (FRP5) following conservative dose-escalation strategies are currently underway for a variety of HER2+ malignancies by other investigators (clinicaltrials.gov identifiers NCT01109095, NCT00889954, and NCT00902044).

A variation on the CAR T-cell theme are antibody-coupled T-cell receptor (ACTR) therapeutics, which use CD16A (FCγRIIIA) to bind to Fc regions of tumour-specific IgG (see eg, WO2015/058018). The aim is to enable more control of CAR T-cell activity in vivo by titrating IgG administered to patients. The CD16 binding sites of the CAR-T-cells may be free, however, to also bind to endogenous IgG of the patients and this reduces the attractiveness of the approach. The approach also needs to consider the inherently long half-life of IgG in the body (around 20 days for IgG in man), which may limit control of CAR-cell activity. Ongoing studies may assess the risk of this. It would be desirable to provide an alternative way to control immune cell-based therapies, like CAR-T-cell approaches, in order to avoid potential complications of using IgG to control activity.

### STATEMENT OF INVENTION

The invention provides a solution by using novel engineered immune cells with small multi-specific fragment-based approaches as switches that do not rely on Fc engagement and which provide for flexible tailoring by readily adapting switch half-lives. One type of this novel approach uses constructs that we call “Chimaeric Antigen Ligands” (CALs) and these are carried on CAL T-cells and other immune cells.

The invention also provides embodiments that, contrary to the art, see benefits in the relatively short serum half-lives of binding fragment approaches. The invention thus improves upon existing T-cell engager antibody approaches in the art (such as BiTEs™ from Amgen) and improves upon the use of Ig for CAR-cell control. Several other advantages are provided by the approaches of the invention, as explained below.

The invention also provides immune cells, CARs, CALs, transplants and methods for Precision Immunotherapy that is tailored to humans and human cells by matching natural human genotypic and phenotypic variation.

To this end, the invention provides the following configurations.

In a first configuration:—
A method of targeting an immune cell to a target cell, the method comprising
A. Providing a bridging agent, wherein the agent is a multi-specific antigen binding fragment comprising
   i. a first antigen binding site that specifically binds a first target antigen; and
   ii. a second antigen binding site that specifically binds a second target antigen;
B. Providing a chimaeric antigen ligand (CAL)-immune cell, wherein the immune cell comprises a transmembrane ligand, the ligand comprising an engineered combination of
   iii. an extracellular moiety comprising the second antigen, wherein the second antigen is linked to a transmembrane domain; and
   iv. an intracellular moiety comprising a first signaling domain for intracellular signaling when the agent binds to the second antigen;
C. Combining the CAL-immune cell and bridging agent with the target cell, the target cell comprising said first target antigen, wherein the first antigen is an extracellular antigen,
   v. whereby the bridging agent binds to the first and second antigens to target the immune cell to the target cell,
   vi. thereby triggering intracellular signaling in the immune cell to regulate immune cell activity.

This enables embodiments wherein the bridging agent has a human serum half-life that is less than the human serum half-life of IgG, thereby enabling finer control than hitherto been possible with previous CAR-T approaches and enabling the possibility to avoid reliance on Fc interaction (which may not readily distinguish from the patient's own antibody Fc regions).

In a second configuration:—
A chimaeric antigen ligand (CAL)-immune cell for targeted binding to an antigen-specific agent,
A. wherein the agent is a multi-specific antigen binding fragment comprising
   i. a first antigen binding site that specifically binds a first target antigen; and
   ii. a second antigen binding site that specifically binds a second target antigen;
B. wherein the CAL-immune cell comprises a transmembrane ligand, the ligand comprising an engineered combination of
   iii. an extracellular moiety comprising the second antigen, wherein the second antigen is linked to a transmembrane domain; and
   iv. an intracellular moiety comprising a first signalling domain for intracellular signalling when the agent binds to the second antigen;
C. wherein when the CAL-immune cell and bridging agent are combined with a target cell, the target cell comprising said first target antigen, wherein the first antigen is an extracellular antigen,
   v. the bridging agent binds to the first and second antigens to target the immune cell to the target cell,
   vi. thereby triggering intracellular signalling in the immune cell to regulate immune cell activity.
      In a third configuration:—

The CAL-immune cell or a transplant comprising a plurality of such cells, for use in a method of treating or reducing the risk of a disease or condition (eg, a cancer) in a human, wherein the method comprises administering the CAL-cell and said bridging agent to the human; wherein the CAL-cell and a target cell of the human are combined and bridged by the bridging agent, thereby up-regulating signalling in the CAL-cell to enhance target cell cytoxicity (eg, ADCC-mediated killing activity) of the CAL-cell, thereby treating or reducing the risk of said disease or condition in the human.

In a fourth configuration:—

The CAL-immune cell or a transplant comprising a plurality of such cells, for use in a method of treating or reducing the risk of a disease or condition (eg, an autoimmune disease, GvHD or allogenic transplant rejection) in a human, wherein the method comprises administering the CAL-cell and said bridging agent to the human; wherein the CAL-cell and a target cell of the human are combined and bridged by the bridging agent, thereby up-regulating signalling in the CAL-cell to reduce cytoxicity (eg, ADCC-mediated killing activity) of the CAL-cell, thereby treating or reducing the risk of said disease or condition in the human.

In a fifth configuration:—
A method of targeting an immune cell to a target cell, the method comprising
A. Providing a bridging agent, wherein the agent is a multi-specific binding fragment comprising
   i. a first binding moiety; and
   ii. a second binding moiety;
B. Providing an immune cell, wherein the immune cell expresses a transmembrane protein comprising an engineered combination of
   iii. ‘an extracellular part comprising a third binding moiety that is linked to a transmembrane domain; wherein the second and third moieties form a specific binding pair (SBP1) wherein one moiety specifically binds to the other moiety; and
   iv. an intracellular part comprising a first signaling domain for intracellular signaling when the second and third moieties bind together;
C. Combining the immune cell and bridging agent with the target cell, the target cell comprising a fourth binding moiety, wherein the fourth moiety is extracellular,
   v. whereby the first and fourth moieties form a specific binding pair (SBP2) wherein one moiety specifically binds to the other moiety to target the immune cell to the target cell,
   vi. wherein the second and third moieties bind together thereby triggering intracellular signaling in the immune cell to regulate immune cell activity; and
D. wherein the molecular weight of the bridging agent is no more than 125 kDa.

This is beneficial to harness serum half-lives for the bridging agents that are less than the half-life of Ig (having a size of 150 kDa).

In a sixth configuration:—
An immune cell for targeted binding to an antigen-specific agent,
A. wherein the agent is a multi-specific binding fragment comprising
   i. a first binding moiety; and
   ii. a second binding moiety;
B. wherein the immune cell expresses a transmembrane protein comprising an engineered combination of
   iii. an extracellular part comprising a third binding moiety that is linked to a transmembrane domain; wherein the second and third moieties form a specific binding pair (SBP1) wherein one moiety specifically binds to the other moiety; and
   iv. an intracellular part comprising a first signaling domain for intracellular signaling when the second and third moieties bind together;
C. wherein when the immune cell and bridging agent are combined with a target cell (the target cell comprising a fourth binding moiety, wherein the fourth moiety is extracellular),
   v. the first and fourth moieties form a specific binding pair (SBP2) wherein one moiety specifically binds to the other moiety to target the immune cell to the target cell;
   vi. the second and third moieties bind together thereby triggering intracellular signaling in the immune cell to regulate immune cell activity; and
D. wherein the molecular weight of the bridging agent is no more than 125 kDa.
   In a seventh configuration:—
   A human immune cell comprising an engineered transmembrane protein,

wherein the protein comprises
A. an extracellular moiety comprising one or more ligand binding domains or one or more ligand domains;
B. a transmembrane moiety; and
C. an intracellular moiety comprising a first signaling domain (SD1);
wherein
D. the SD1 of the engineered protein is encoded in the cell by a first nucleotide sequence (S1) comprising a human single nucleotide polymorphism (SNP1) that encodes an amino acid residue (R1) of SD1;
E. the genome of the cell comprises a second nucleotide sequence (S2) comprising SNP1 and encoding a second signaling domain (SD2), wherein the second signaling domain is (i) identical to SD1 and comprises R1 or (ii) a naturally-occurring variant of SD1 and comprises R1; and
F. wherein S2 is an endogenous genomic sequence of the cell and SNP1 is a non-synonymous SNP.
   A human immune cell comprising an engineered transmembrane protein,
   wherein the protein comprises
A. an extracellular moiety comprising a first antigen or ligand domain;
B. a transmembrane moiety; and
C. an intracellular moiety comprising a first signaling domain;
   wherein
D. the first antigen or ligand domain of the engineered protein is encoded in the cell by a first nucleotide sequence (S1) comprising a human single nucleotide polymorphism (SNP1) that encodes an amino acid residue (R1) of the antigen or ligand domain;
E. the genome of the cell comprises a second nucleotide sequence (S2) comprising SNP1 and encoding a second antigen or ligand domain, wherein the second antigen or ligand domain is (i) identical to the first antigen or ligand domain respectively and comprises R1 or (ii) a naturally-occurring variant of the first antigen or ligand domain respectively and comprises R1; and
F. wherein S2 is an endogenous genomic sequence of the cell and SNP1 is a non-synonymous SNP.
   A human immune cell for use used in a method of treating or reducing the risk of a disease or condition (eg, as disclosed herein, eg, a cancer or autoimmune disease), wherein the method comprises administering the immune cell to a human patient, the immune cell comprising an engineered transmembrane protein,
   wherein the protein comprises
A. an extracellular moiety comprising one or more ligand binding domains or one or more ligand domains;
B. a transmembrane moiety; and
C. an intracellular moiety comprising a first signaling domain (SD1);
   wherein
D. SD1 of the engineered protein is encoded in the cell by a first nucleotide sequence (S1) comprising a human single nucleotide polymorphism (SNP1) that encodes an amino acid residue (R1) of SD1;
E. the genome of the human comprises a second nucleotide sequence (S2) comprising SNP1 and encoding a second signaling domain (SD2), wherein SD2 is (i) identical to SD1 and comprises R1 or (ii) a naturally-occurring variant of SD1 and comprises R1;
F. wherein S2 is an endogenous genomic sequence of the human and SNP1 is a non-synonymous SNP; and
G. wherein the human genome comprises S2 before said administration of the immune cell; and
H. wherein the method treats or the risk of the disease or condition in the human.
   A human immune cell for use used in a method of treating or reducing the risk of a disease or condition (eg, as disclosed herein, eg, a cancer or autoimmune disease), wherein the method comprises administering the immune cell to a human patient, the immune cell comprising an engineered transmembrane protein,

wherein the protein comprises
A. an extracellular moiety comprising a first antigen or ligand domain;
B. a transmembrane moiety; and
C. an intracellular moiety comprising a first signaling domain;
   wherein
D. the first antigen or ligand domain of the engineered protein is encoded in the cell by a first nucleotide sequence (51) comprising a human single nucleotide polymorphism (SNP1) that encodes an amino acid residue (R1) of the antigen or ligand domain;
E. the genome of the human comprises a second nucleotide sequence (S2) comprising SNP1 and encoding a second antigen or ligand domain, wherein the second antigen or ligand domain is (i) identical to the first antigen or ligand domain respectively and comprises R1 or (ii) a naturally-occurring variant of the first antigen or ligand domain respectively and comprises R1;
F. wherein S2 is an endogenous genomic sequence of the human and SNP1 is a non-synonymous SNP; and
G. wherein the human genome comprises S2 before said administration of the immune cell; and
H. wherein the method treats or the risk of the disease or condition in the human.
   In an eighth configuration:—
   A human immune cell comprising an engineered transmembrane protein,

wherein the protein comprises
A. an extracellular moiety comprising one or more ligand binding domains or one or more ligand domains;
B. a transmembrane moiety; and
C. an intracellular moiety comprising a first signaling domain;
D. wherein the first signaling domain is a CD3 intracellular domain selected from a CD3ζ (CD3-zeta) domain and a CD3n (CD3-eta) domain, and comprises at least 50 amino acid residues selected from the group consisting of V53, K54, F55, R57, S58, D60, Y64, Q65, Q68, L71, E74, L75, N76, L77, G78, R80, E81, Y83, L86, R89, G91, P94, E95, G98, K99, R102, Q107, G109, Y111, N112, E113, L114, Q115, K116, D117, K118, M119, E121, A122, Y123, 5124, E125, 1126, G127, G130, R134, G135, H138, D139, L141, Y142, Q143, G144, S146, T147, T149, K150, D151, D154, H157, M158, Q159, L161 and P162 (position numbers correspond to positions of SEQ ID NO: 1); and
E. wherein the genome of the cell comprises an endogenous nucleotide sequence encoding a second signaling domain, wherein the second domain is a CD3ζ (CD3-zeta) domain or a CD3η (CD3-eta) domain comprising at least 40 (eg, 45 or all) of said selected residues.
   A human immune cell for use used in a method of treating or reducing the risk of a disease or condition (eg, as disclosed herein, eg, a cancer or autoimmune disease), wherein the method comprises administering the immune cell to a human patient, the immune cell comprising an engineered transmembrane protein,
   wherein the protein comprises
A. an extracellular moiety comprising a first antigen or ligand domain;
B. a transmembrane moiety; and
C. an intracellular moiety comprising a first signaling domain;
   wherein
D. wherein the first signaling domain is a CD3 intracellular domain selected from a CD3ζ (CD3-zeta) domain and a CD3η (CD3-eta) domain, and comprises at least 50 amino acid residues selected from the group consisting of V53, K54, F55, R57, S58, D60, Y64, Q65, Q68, L71, E74, L75, N76, L77, G78, R80, E81, Y83, L86, R89, G91, P94, E95, G98, K99, R102, Q107, G109, Y111, N112, E113, L114, Q115, K116, D117, K118, M119, E121, A122, Y123, 5124, E125, 1126, G127, G130, R134, G135, H138, D139, L141, Y142, Q143, G144, S146, T147, T149, K150, D151, D154, H157, M158, Q159, L161 and P162 (position numbers correspond to positions of SEQ ID NO: 1); and
E. wherein the genome of the human comprises an endogenous nucleotide sequence encoding a second signaling domain, wherein the second domain is a CD3ζ (CD3-zeta) domain or a CD3η (CD3-eta) domain comprising at least 40 (eg, 45 or all) of said selected residues;
F. wherein the method treats or the risk of the disease or condition in the human.
   In a ninth configuration:—
   A human immune cell comprising an engineered transmembrane protein,

wherein the protein comprises
A. an extracellular moiety comprising a first antigen or ligand domain;
B. a transmembrane moiety; and
C. an intracellular moiety comprising a first signaling domain (SD1);
D. wherein SD1 is a CD28 intracellular domain comprising at least 13, 14, 15, 16, 17 or 18 amino acid residues selected from the group consisting of R180, 5181, K182, R183, 5184, R185, L186, D190, Y191, N193, P196, P199, T202, K204, Q207, F215, A217 and Y218 (position numbers correspond to positions of SEQ ID NO: 13); and
E. wherein the genome of the cell comprises an endogenous nucleotide sequence encoding a second signaling domain (SD2), wherein SD2 is a CD28 intracellular domain comprising at least 10 (or 11, 12 or 13) of said selected residues.

A human immune cell for use used in a method of treating or reducing the risk of a disease or condition (eg, as disclosed herein, eg, a cancer or autoimmune disease), wherein the method comprises administering the immune cell to a human patient, the immune cell comprising an engineered transmembrane protein,

wherein the protein comprises
A. an extracellular moiety comprising a first antigen or ligand domain;
B. a transmembrane moiety; and
C. an intracellular moiety comprising a first signaling domain (SD1);
   wherein
D. wherein SD1 is a CD28 intracellular domain comprising at least 13, 14, 15, 16, 17 or 18 amino acid residues selected from the group consisting of R180, 5181, K182, R183, 5184, R185, L186, D190, Y191, N193, P196, P199, T202, K204, Q207, F215, A217 and Y218 (position numbers correspond to positions of SEQ ID NO: 13);
E. wherein the genome of the human comprises an endogenous nucleotide sequence encoding a second signaling domain (SD2), wherein SD2 is a CD28 intracellular domain comprising at least 10 (or 11, 12 or 13) of said selected residues; and
F. wherein the method treats or the risk of the disease or condition in the human.
   In a tenth configuration:—
   A human immune cell comprising an engineered transmembrane protein,

wherein the protein comprises
A. an extracellular moiety comprising a first antigen or ligand domain;
B. a transmembrane moiety; and
C. an intracellular moiety comprising a first signaling domain (SD1);
D. wherein SD1 is a 4-1BB intracellular domain comprising at least 10, 11, 12, 13, 14 or all of the residues selected from the group consisting of R215, R217, K218, Y222, P227, M229, V232, Q236, D239, C241, R244, E247, E250, G252 and C253 (position numbers correspond to positions of SEQ ID NO: 16); and
E. wherein the genome of the cell comprises an endogenous nucleotide sequence encoding a second signaling domain (SD2), wherein SD2 is a 4-1BB intracellular domain comprising at least 8 (or 9 or 10) of said selected residues.
   A human immune cell for use used in a method of treating or reducing the risk of a disease or condition (eg, as disclosed herein, eg, a cancer or autoimmune disease), wherein the method comprises administering the immune cell to a human patient, the immune cell comprising an engineered transmembrane protein,
   wherein the protein comprises
A. an extracellular moiety comprising a first antigen or ligand domain;
B. a transmembrane moiety; and
C. an intracellular moiety comprising a first signaling domain (SD1);
   wherein
D. wherein SD1 is a 4-1BB intracellular domain comprising at least 10, 11, 12, 13, 14 or all of the residues selected from the group consisting of R215, R217, K218, Y222, P227, M229, V232, Q236, D239, C241, R244, E247, E250, G252 and C253 (position numbers correspond to positions of SEQ ID NO: 16); and
E. wherein the genome of the human comprises an endogenous nucleotide sequence encoding a second signaling domain (SD2), wherein SD2 is a 4-1BB intracellular domain comprising at least 8 (or 9 or 10) of said selected residues.
F. wherein the method treats or the risk of the disease or condition in the human.

The invention also provides transplants, cell populations, kits and devices comprising CAL-immune cells and/or bridging agents of the invention

### DETAILED DESCRIPTION

The invention in its various aspects is based on the following considerations—
Use of relatively small, multi-specific ligand binding fragments for switching and controlling immunotherapy (with applicability to CAL- and CAR-immune cells);
The possibility to re-purpose existing mAb and Ig or non-Ig antigen binding fragments by combination with immune cell-mediated activity, for example enabling new modalities based on FDA or EMA-approved mAbs;
The provision of CAL-cells as novel types of immune effector cells in vivo and ex vivo;
The utility of CAR- and CAL-cell expansion in vivo to reduce reliance on administration of high, toxic levels of multi-specific fragments as seen in the art;
The utility of CAR- and CAL-cell expansion in vivo to reduce reliance on prolonged (eg, continuously pumped) administration of multi-specific fragments as seen in the art;
The possibility of better tumour penetration of fragments as opposed to mAbs for addressing solid tumours;
The possibility to harness tumour infiltrating lymphocytes (TILs) in combination with fragment approaches to address solid tumours, including the possibility to use lower dose co-administration of immune up-regulators such as IL-2; and
The observation of natural human polymorphic variation in CAR and CAL binding domains and matching to human and human cell genotypic and phenotypic variation to provide what we have called “Precision Immunotherapy”.

### Utility of Multi-Specific Fragments

Use of a bridging agent with multiple ligand binding sites according to the invention enables the use of bi-, tri- and multi-specific fragments (eg, antibody-based fragments as well known in the art) of relatively small size that have half-lives that are relatively short (much shorter than IgG, which has an average half-life in human serum of around 20 days). In this way, the invention enables ready titration of the bridging agent to act as a switch for triggering signaling changes in the immune cells of the invention. This provides, for example, a titratable way of readily changing the activity of immune cells in immunotherapy, such as CAR-cells (eg, CAR T-cells, CAR NK cells and CAR TIL cells) or CAL-cells (eg, CAL T-cells, CAL NK cells and CAL TIL cells) that are administered to a patient, thereby enabling a convenient way of controlling potent the therapy. This helps to address concerns in the art of how to avoid unwanted over-activity of CAR-cell therapies in patients. It may also be advantageous to use relatively small fragments as bridging agents to enable closer proximity between target and immune cells that have been bridged according to the invention.
Existing bispecific fragment technologies, such as BiTEs™ (Amgen) or other bispecific T-cell engagers, can be readily used as bridging agents according to the invention, thereby making the invention convenient (especially where the agent has already been FDA or EMA approved). An example is blinatumomab (Blincyto™ from Amgen), which has an scFv binding site for human CD3δ linked to an scFv binding site for human CD19 (a tumour associated antigen, TAA). Blinatumomab has been approved for certain cancer treatments where CD3 engagement activates T-cell killing of tumour cells.

Reference is made to: Front Oncol. 2014; 4: 63; Published online 2014 Mar. 31; doi: 10.3389/fonc.2014.00063; PMCID: PMC3978294; “Blinatumomab, a Bi-Specific Anti-CD19/CD3 BiTE® Antibody for the Treatment of Acute Lymphoblastic Leukemia: Perspectives and Current Pediatric Applications”; Lindsey M. Hoffman and Lia Gore, which describes the relatively short half-life of this BiTE™ (approximately 2 hours) and administration of the drug by continuous infusion by pump, requiring hospital admission. As exemplified by blinatumomab, therapy using such small fragments is perceived to be hampered by the relatively short half-lives of the agents; in the case of blinatumomab to address this large doses are administered to patients by an implanted pump that continuously pumps the agent over 4 weeks. This is inconvenient as it requires installation of a pump by medical staff and the patient is inconveniently hooked up to this 24 hours a day during the treatment period. Furthermore, there can be adverse side effects of administering relatively large doses of multi-specific fragments generally.

Contrary to the long-held attitude in the art, the invention instead actually sees utility in the relatively short half-lives of bi-, tri- and multi-specific fragment approaches; the inventor has realized that such agents can be used as a readily controllable switch for more fine control of activated T-cell, NK-cell, TIL etc killing of target cells. In particular, in an example this allows for more fine-tuning of engineered immune cell activity, such as ADCC-like activity, in patients, which prior has not been possible with earlier CAR-T approaches. In doing so, benefits of CAR-cell-mediated and bispecific T-cell engager (eg, BiTE™)-mediated treatment of disease (eg, cancer) can be realized in a more controlled fashion than previously possible. An advantage, therefore, is that immune cell-based therapies can be more readily regulated, unlocking even greater potential for such ground-breaking strategies. Also, perceived undesirable consequences of small multi-specific fragment treatments are lessened, thereby enabling the potential of lower dosing of engager agents and reduced dependence on continual drug pumping over extended periods as presently seen, eg, with blinatumomab and other small multi-specific fragment approaches.

Furthermore, rather than relying on large enough amounts of multi-specific fragment in the patient to promote target cell killing, as with blinatumomab, the invention instead is able to harness the expansion capability of engineered immune cells to provide an amplified killing or regulation of target cells. For this reason too, the amount of bridging agent may be reduced in certain settings, thereby reducing the risk of side effects and off-target killing or undesirable regulation of normal cells. The ability to use reduced amounts of the bridging agent of the invention may be advantageous in reducing unwanted targeting of normal cells also expressing low levels of the first target antigen. Thus, the invention may be particularly useful when the first antigen is present at higher levels on target (eg, tumour) cells than on normal cells, as stringency of targeting may be controllable to a certain extent by lower titration of bridging agent. With fragments such as blinatumomab, whose activity has been suggested to be dependent upon serial lysis (necessitating continuous drug administration), it may be possible instead with the present invention to break reliance on such mechanisms, as the invention builds in the possibility for cell-mediated cytotoxicity (eg, ADCC-like activity) by the immune cells according to the invention, which effect may benefit from the ability of immune cell expansion in the patient.

### Chimaeric Antigen Ligand Receptors (CALs) & CAL-Immune Cells

It will be realised that the present invention deviates from prior approaches—not only in seeing utility in the short half-lives of fragment approaches—but also in certain configurations by swapping antigen ligand/binding site pairings to provide CAL-immune cells and uses of these. Prior art CAR-T and CAR-NK approaches rely on the provision of the antigen binding site as an extracellular feature of the immune cell's “chimaeric antigen receptor” (CAR); in contrast the present invention relies upon the provision of the antigen ligand itself as the cell-surface feature of the immune cell, the binding site instead being located on the bridging agent. This not only conveniently provides the advantage of using off-the-shelf multi-specific fragments (as explained above), but advantageously it is possible to use self-antigen as the extracellular antigen of the immune cell signalling complex (eg, self-version of a CD3 extracellular domain or other domain normally found on T-, NK or other relevant immune cells of humans). We call these “Chimaeric Antigen Ligands” (CAL) to distinguish them from CARs. Example immune cells of the invention are CAL T-cells, CAL NK cells and CAL TIL cells. The ability to use self-antigen reduces the risk of the CAL-cells being targeted and cleared by the patient's immune system, which has utility for autologous or allogeneic cell transplants; with CAR-cells there is the risk that the antigen binding site of the receptor may comprise immunogenic epitopes and thus may be a target for the patient's own immune system, thereby reducing efficacy. The use of CALs enables the extracellular antigen to be provided by a protein type (eg, CD3γ, δ or ε) that naturally occurs on the surface of immune cells (eg, T-cells) of the patient, which may be useful for compatibility with the patient. Knock-out of nucleotide sequences in the CAL-cell can be used to prevent expression of one or more endogenous domains or proteins of the TCR-CD3 signalling complex, thereby directing signalling instead to the CAL of the invention (or CAR-cell for other configurations of the invention that use CAR-cells with the bridging agent).

### Bridging Agents

There is a further advantage of the invention: it is possible to adjust the affinity of binding of the bridging agent to the CAL in order to tune cytotoxicity or other regulation caused by the engineered immune cells. With prior CAR-T approaches, the binding interaction is dictated by a need to produce relatively high binding affinity of the extracellular binding site of the CAR to its target antigen on a tumour cell. With the approach of the present invention, however, this consideration is transferred to the first binding site of the bridging agent (first binding moiety in the 5^{th} and 6^{th} configurations of the invention) and one is thus free to choose the appropriate affinity to bind strongly to tumour cells, for example. This allows for flexibility in the binding interaction between the second binding site of the bridging agent (second binding moiety in the 5^{th} and 6th configurations of the invention) and the second target antigen comprised by the CAL (third binding moiety in the 5^{th} and 6^{th} configurations of the invention comprised by the transmembrane protein). Thus, it is possible, for example, to choose a binding strength (eg, medium KD and/or medium Koff) to dial down or up the activity of the engineered immune cells as they expand and are activated when bridged to the target cells. The invention, therefore, allows for more fine tuning than has been possible with prior CAR-T approaches, since the invention enables the skilled addressee to purposely balance half-life and binding affinities to the situation at hand. Binding affinities at two binding sites of the agent can be balanced, which allows for finer tuning than choosing the affinity of one binding site only (as with CARs). The use of fragments according to the invention (such as those comprising antibody or non-Ig scaffold domains, which are advantageously modular and readily and cheaply produced by E coli and other systems) provides a straightforward way to tune the binding affinities, as this allows one to use repertoire selection approaches such as phage or yeast display of binding members which is conveniently routine and well developed in the art. The invention is also amenable to using binding sites of well-established, existing monoclonal antibody therapeutics that have been approved and shown to be tolerated in patients. By combining one or more of such binding sites (eg, binding sites from first and second, different antibodies) with a CAL binding site (or with a ligand to which a CAR specifically binds), an appropriate bi- or tri-specific bridging agent can be made where specific, affinities have been purposely designed for the treatment setting at hand. Tri-specificity (or higher order multi-specificity) is useful, for example, for targeting at least 2 different cell surface targets on target cells (eg, tumour cells), where those targets are not comprised on normal cells or are present together at lower levels than on tumour cells. The invention, therefore, provides such a bridging agent in combination with the engineered immune cells, and use for treating or reducing the risk of a disease or condition as described herein.

Furthermore, unlike ACTR approaches which rely on bulky IgG co-administration, the present invention enables use of much smaller binding fragments and thus, the molecular weight of the bridging agent can be chosen to be much smaller than that of IgG (which is 150 kDa). This provides the possibility of lower amounts for patient dosing and also lower potential cost of goods to produce the bridging agent. Closer proximity of bridged cells and ease of manufacture by bacterial (eg, E coli) or yeast (eg, Picchia) as discussed above are further benefits over ACTR approaches using IgG.

Sizes of example binding fragments are shown in the following table. The binding agent of the invention can be any multi-specific binding fragment shown in that table or comprising any such fragment, wherein the agent has a size of less than an Ig. For example, the molecular weight of the agent is less than 125, 120, 115, 110, 100, 90, 80, 70, 60, 50 or 40 kDa.

| | Molecular Weight Size Range | |
|---|---|---|
| Antigen Binding Fragment | (kDa)* | Valence |
| ZIP miniantibody | Up to 70 | 2 |
| Diabody | | |
| (scFv)₂/BITE ™ | | |
| Sc-Diabody | | |
| Barnase-barstar dimer | 70-90 | |
| Minibody | | |
| (Fab)₂ | 90-120 | |
| sc(Fab)₂ | | |
| scFv-Fc | | |
| Triabody | 70-90 | 3 |
| Trimerbody | 100-130 | |
| Tribody | | |
| Tribi-minibody | | |
| Collabody | | |
| Barnase-barstar trimer | 130-140 | |
| (scFv-TNF_{α})₃ | | |
| Tandab | 110-130 | 4 |
| [sc(Fv)₂]₂ | | |
| Tetrabody | | |
| (scFv-p53)₄ | | |
| Di-diabody | | |

*indicates size range in which the fragment is found

See figure in “Multivalent antibodies: when design surpasses evolution”, Ángel M. Cuesta et al, Cell, Volume 28, Issue 7, p 355-362, July 2010., which is incorporated herein by reference.

### Treating Solid Tumours & Tumour Infiltrating Lymphocytes (TILs)

In an embodiment, a benefit of the invention harnesses tumour penetrative capacities of small multi-specific binding fragments, which find utility for example for treating solid tumours. Such fragments, such as ScFv-based fragments retain the binding specificity of the parent antibody and offer several advantages compared to full-length mAbs. For instance, these fragments can penetrate more rapidly into tumours compared to an intact antibody (see, eg, Chowdhury, P. S.; Viner, J. L.; Beers, R.; Pastan, 1. “Isolation of a high-affinity stable single-chain Fv specific for mesothelin from DNA-immunized mice by phage display and construction of a recombinant immunotoxin with anti-tumour activity”, Proc. National. Acad. Sci. USA 1998, 95, 669-674; and Deckert, P. M. “Current constructs and targets in clinical development for antibody-based cancer therapy”, Curr. Drug Targets 2009, 10, 158-175). It has been argued that the optimal tumour-targeting fragment would be a diabody (55-60 kDa) combining high tissue penetration, target retention and rapid blood clearance (Robinson, M. K. et al, “Quantitative immuno-positron emission tomography imaging of HER2-positive tumour xenografts with an iodine-124 labeled anti-HER2 diabody”, Cancer Res. 2005, 65, 1471-1478; and Sundaresan, G et al, “1241-labeled engineered anti-CEAcea minibodies and diabodies allow high-contrast, antigen-specific small-animal PETimaging of xenografts in athymic mice”, J. Nucl. Med. 2003, 44, 1962-1969). The small sizes of fragments, though a desirable property for tissue penetration, such as in cancer therapy, also leads to a short in vivo half-life, limiting the exposure of the target molecule to the fragment. In the present invention, the relatively short half-life is advantageously used to enable finer switching of immunotherapy. Whilst not wishing to be bound by any particular theory, the capture of bridging agent by immune-cells according to the invention in vivo at the site of cancer cells may prolong the persistence of the agent in the microenvironment of the cancer, thus compensating for low general systemic half-life. For similar reasons, it is useful in some embodiments to use TILs as the basis of immune cells of the invention as these types of cells have been shown to infiltrate solid tumours, and this together with captured bridging agent can be beneficial for treating solid tumours using the present invention. Thus, in one embodiment, the immune cell of the invention is a TIL for treating or preventing a solid tumour in a patient (eg, a human). Example agents for use with such a TILs (or a transplant comprising a plurality of such TILs) are bi- and tri-specific antigen binding fragments comprising two or three scFv binding sites. As discussed further below, the benefit of existing mAb solid tumour therapies can be re-deployed in the present invention by using an antibody VH/VL binding site of such a mAb as the first binding site (or first binding moiety) in the bridging agent of the invention (eg, provided as an scFv). Thus, the invention provides an immune cell (eg, CAL-TIL or CAR-TIL) of the invention in combination with a bridging agent of the invention (either mixed together; or separately and comprised by a kit) for treating or preventing a solid tumour in a patient (eg, a human), wherein the binding sites of the agent are optionally scFv binding sites linked by a linker.
In an example of these aspects of the invention (or any other aspects herein), the agent is a diabody (eg, size=55-65 kDa), triabody (eg, size=85-95 KDa) or tetrabody (eg, size=115-125 kDa). Diabodies outperform monomeric scFvs with a better tumour blood ratio (Appl Microbiol Biotechnol. 2013 May; 97(9):3855-63. doi: 10.1007/s00253-012-4632-9. Epub 2012 Dec. 19, “Production and characterization of a CD25-specific scFv-Fc antibody secreted from Pichia pastoris”, Wan L et al). Thus, recombinant antibodies of 60-100 kDa have been found to display efficient tumour penetration and fast circulation clearance compared to the intact antibody and are thus better suited for in vivo tumour targeting. Thus, in an example of any aspect herein the size of the agent is from 60 to 100 kDa.
In an alternative, the CAL-cell(s) are CAL T-cells or CAL NK cells or a mixture of two or three of CAL TIL, CAR T-cells and CAR NK cells. In an alternative, the CAR-cell(s) are CAR T-cells or CAR NK cells or a mixture of two or three of CAR TIL, CAR T-cells and CAR NK cells.
Solid tumours are made up of a variety of components, including malignant cells and endothelial, structural and immune cells. Cancer cells are able to shape the microenvironment to satisfy their own metabolic and immunological needs. In opposition to this, tumour-infiltrating lymphocytes (TILs) are recruited into the tumour in an attempt to control its growth. Evidence is accumulating to show that the quantity of TILs at diagnosis is associated with prognosis. TILs from a patient can be manipulated to be used as treatment for that patient's cancer.
Adoptive cell therapy (ACT) with TILs is an effective strategy for the treatment of cancers, such as metastatic melanoma. The technique involves the generation of TIL cultures from a patient's melanoma biopsy and the rapid expansion in an interleukin-2 (IL-2)-containing medium of lymphocytes displaying high antitumour activity. The TILs are subsequently reintroduced into the same patient following lymphodepletion and in the presence of high-dose IL-2. Despite having been described over a decade ago, ACT with TILs using lymphodepletion has not been as widely adopted as might be expected given its apparent efficacy. A contributing factor to this may be the toxicity associated with high-dose IL-2 which, although generally transient, can be severe.
Thus, in one embodiment the invention the immune cell (eg, CAL-immune cell or CAR-immune cell) is a TIL. In an example, the invention provides a method of treating or reducing the risk of cancer (eg, a solid tumour or melanoma) in a patient (eg, a human), wherein the method comprises administering to the human a CAL- or CAR-TIL and bridging agent of the invention and optionally IL-2. In an example, the method comprises administering an autologous or allogeneic TIL transplant to the human, wherein the transplant comprises a plurality of CAL-TILs or CAR-TILs of the invention. By using such TIL therapy, with its potential for cell expansion in vivo and cytotoxicity directed to cancer cells, the invention provides in an example the use of a lower dose of IL-2 than used previously. Thus, in an example, low or intermediate dose IL-2 (as understood by the skilled person in the field of trastuzumab trials) is administered to the human. In an example, low-dose IL-2 is 1 million IU/m(2) or less daily. In an example, the IL-2 is administered subcutaneously (SC)). In an alternative or in addition to IL-2 administration, one, two or all of IL-12, IL-15 and IL-21 is administered to the human (eg, as a low dose). See, for example, previous trials with low and intermediate IL-2 doses: Breast Cancer Res Treat. 2009 September; 117(1):83-9. doi: 10.1007/s10549-008-0251-7. Epub 2008 Dec. 3, “A phase II trial of trastuzumab in combination with low-dose interleukin-2 (IL-2) in patients (PTS) with metastatic breast cancer (MBC) who have previously failed trastuzumab”, Mani A et al; and J Immunother Cancer. 2014; 2 (Suppl 3): Published online 2014 Nov. 6. doi: 10.1186/2051-1426-2-53-P1, PMCID: PMC4288376, “Adoptive cell therapy with tumour infiltrating lymphocytes and intermediate dose IL-2 for metastatic melanoma”, Rikke Andersen et al. In an embodiment of the TIL-based method of the invention, the method treats or reduces the risk of cancer in a patient (eg, a human), wherein the patient has undergone lymphodepletion before administration of the immune cell(s) of the invention to the patient.

### Triple-Negative Breast Cancer (TNBC) & HER2 Targeting by the Bridging Agent

Several studies support the suggestion that breast cancer is immunogenic. Data from an adjuvant trial in triple-negative breast cancer (TNBC) were used to investigate the prognostic implications of TILs in TNBC and associations with trastuzumab benefit in HER2-overexpressing disease. There was a positive association between the amount of TILs present at diagnosis and prognosis in TNBC. There was also an interaction between higher levels of TILs and increased benefit from trastuzumab. Thus, in one example, the first antigen is HER2 (or this is the fourth binding moiety in the 5^{th} and 6^{th} configuration). In an example, the first binding site (first binding moiety in the 5^{th} and 6^{th} configurations) comprises a VH/VL binding site of trastuzumab or Herceptin™, eg, wherein the binding agent comprises a first scFv, wherein the scFv binds HER2 (the first antigen, or the fourth binding moiety) and comprises VH-linker-VL, wherein the VH and VL are variable domains of trastuzumab or Herceptin™. In an example, the linker of any scFv herein is a (G₄S)ₙ linker, wherein n=2 or more (eg, 3, 4, 5 or 6). In an example, the immune cell (eg, CAL-cell of the invention or CAR-TIL or CAL-TIL) or method of the invention is for treating or reducing the risk of breast cancer in a human (eg, triple negative breast cancer). In an example, the method comprises administering a cell transplant to the human, wherein the transplant is autologous or allogeneic and comprises a plurality of TILs of the invention and a bridging agent of the invention, wherein the first binding site (first binding moiety) of the bridging agent specifically binds HER2, eg, wherein the first binding site (first binding moiety) comprises a VH/VL binding site of trastuzumab or Herceptin™, eg, provided as an scFv as described above.

In an example, an aspect provides the CAL-TIL (or CAL-T or CAL-N K) of the invention for use in a method of treating or reducing the risk of breast cancer in a human (eg, triple negative breast cancer), wherein the first antigen is HER2 and optionally the first binding site of the bridging agent comprises a VH/VL binding site of trastuzumab or Herceptin™, wherein the method comprises administering the CAL-cell and bridging agent to the human, wherein breast cancer is treated or the risk of breast cancer is reduced.

In an example, the binding agent comprises an scFv anti-HER2 binding site, optionally wherein the scFv comprises a VH-linker-VL wherein the VH and VL are variable domains of a binding site of trastuzumab or Herceptin™. In an example, the second binding site of the binding agent specifically binds a human CD3 extracellular domain or a CD16 (eg, CD16A) extracellular domain.

In an example, the CAL-cell of the invention is comprised by a transplant comprising a plurality of CAL-TILs (or CAL-T, or CAL-NK cells) of the invention and the transplant is administered to the human to treat or prevent a disease (eg, a cancer, autoimmune disease, transplant rejection of GvHD) or the cell or transplant is for such use.

In an example, the human is a woman; or a man.

In an example, the patient or human has undergone lymphodepletion before administration of the immune cell (eg, CAL-cell) of the invention.

Techniques for producing CARs and CAR T-cells are known and routine in the art, and these can be generally applied to producing CALs and CAL-cells of the invention (eg, see WO2012079000A1; J Immunother. 2009 September; 32(7): 689-702, doi: 10.1097/CJI.0b013e3181ac6138, “Construction and Pre-clinical Evaluation of an Anti-CD19 Chimeric Antigen Receptor”, James N. Kochenderfer et al; also WO 2014012001 for general methods applicable to the present invention). For example, use of electroporation, retroviral vectors or lentiviral vectors—as will be known by the skilled addressee—can be used to introduce nucleotide sequences encoding elements of the CAL of the invention into T-cells, NK cells, TILs or other immune cells to produce the CAL-cells of the invention. Cells isolated from the patient (autologous cell sample) or from another donor (allogeneic sample) can be used to provide ancestor cells that are genetically engineered to include the CAL-encoding sequences. Expansion of cells can be used in the process, as known in the art. For example, after engineering CAL-cells, the cell population can be massively expanded using routine techniques to produce a transplant that is administered (eg, transfused) into the patient. The patient can be a human on non-human animal. Nucleotide sequences for one or more of the CAL elements (eg, for the second antigen and/or first signalling domain) can be cloned or sequenced using a cell obtained from the patient or from another donor.

### Domain Variations & Matching

In an embodiment of the 5^{th} and 6^{th} configuration, the second binding moiety is a ligand (eg, antigen) and the third moiety is a ligand receptor or binding site (eg, VH/VL binding site), eg, wherein the immune cell is a CAR-cell. In an advantageous example, the second binding moiety sequence is SNP-matched for one or more non-synonymous SNPs naturally found in humans in the coding sequence of the second moiety.

In an advantageous example of the 1^{st} to 4^{th} configurations relating to CALs, the second antigen sequence is SNP-matched for one or more non-synonymous SNPs found in humans in the coding sequence of the second antigen (eg, with reference to one or more SNPs found naturally in human CD3 extracellular domain).

Suitable databases for assessing and identifying SNPs are known to the skilled person, such as Ensembl and the 1000 Genomes database. With reference to variation at a particular nucleotide position in an expressible sequence, the skilled person will know that a “non-synonymous SNP” at a particular nucleotide position is a single nucleotide polymorphism at that position wherein natural variation produces different amino acid residue consequences in the encoded protein sequence. Consideration of one or more SNP variations or the corresponding amino acid changes in the bridging agent's second binding moiety (5^{th} and 6^{th} configurations of the invention) or CAL extracellular domain's second antigen (1^{st} to 4^{th} configurations) is useful to match this element of the agent or CAL to mirror the natural SNP variation in equivalent protein encoded by the immune cell of the invention (or an ancestor cell of which this is a progeny) and/or encoded by the genome of the patient. This is particular useful when the genome of the patient receiving the immune cell (or cell transplant) of the invention encodes such matched SNP(s), in which case this increases compatibility of the bridging agent (in the 5^{th} and 6^{th} configurations) or CAL extracellular moiety (in the 1^{st} to 4^{th} configurations) with the immune system of the patient (and this can be useful to reduce immune response against the agent or CAL that otherwise may reduce utility). In an advantageous embodiment, the patient expresses the second binding moiety (5^{th} and 6^{th} configuration) or the second antigen (1^{st} to 4^{th} configurations), ie, a protein whose amino acid sequence is identical to the amino acid sequence of the second moiety of such agent or the second antigen of the CAL. Thus, for example the the second binding moiety (5^{th} and 6^{th} configuration) or the second antigen (1^{st} to 4th configurations) is a CD3γ, δ or ε domain, wherein the recipient of the agent and immune cell according to the invention expresses a matched CD3γ, 6 or ε domain respectively.

Similarly, aspects of the invention match the intracellular signalling domain(s) of the transmembrane protein (eg, CAR or CAL) to help optimise performance inside the engineered immune cell. In this instance SNP matching is used between (i) the nucleotide sequences (non-endogenous sequences, eg, introduced on a lentiviral or retroviral vector) encoding the first (or each) signalling domain of the transmembrane protein and (ii) the endogenous nucleotide sequences of the cell encoding such signalling domains. By matching in this way, the signalling domains are also matched to other components of the endogenous signalling cascades in the cell, to help optimise performance. As used herein “endogenous” refers to any naturally-occurring material in or from or produced inside an organism, cell, tissue or system, for example found in non-engineered cells of a patient that has or will be administered the immune cells (eg, CAR- or CAL-cells) according to the invention or from a donor from which the immune cells are derived.

For example, the first signalling domain is a human CD3 domain and the cell of the invention is a human cell comprising an endogenous nucleotide sequence encoding said human CD3 domain. In an example, the CD3 zeta signaling domain comprises the amino acid sequence of SEQ ID NO: 24 as disclosed in WO2012079000A1, which sequence is explicitly incorporated herein for use in the present invention and possible inclusion in one or more claims herein. In an example, the CD3 zeta signaling domain is encoded by the nucleic acid sequence of SEQ ID NO: 18 as disclosed in WO2012079000A1, which sequence is explicitly incorporated herein for use in the present invention and possible inclusion in one or more claims herein.

For example, the first signalling domain is a human CD28 domain and the cell of the invention is a human cell comprising an endogenous nucleotide sequence encoding said human CD28 domain.

For example, the first signalling domain is a human 4-1BB domain and the cell of the invention is a human cell comprising an endogenous nucleotide sequence encoding said human 4-1BB domain.

For example, the first signalling domain is a human OX40 domain and the cell of the invention is a human cell comprising an endogenous nucleotide sequence encoding said human OX40 domain.

In an example, when the second binding moiety (5^{th} and 6^{th} configurations) or second antigen (1^{st} to 4^{th} configurations) is a CD3γ, 6 or ε domain and the first signalling domain is a CD3ζ domain, the moiety/antigen and domain do not naturally occur together in a single cell (eg, a human wild-type cell or a cell isolated from the patient). In another example, when the second binding moiety or second antigen is a CD3γ, δ or ε domain and the first signalling domain is a CD3ζ domain, the transmembrane protein (eg, CAL or CAR) comprises a further domain that is not a CD3 domain, eg, the further domain is a CD28, CD27, OX40 or 4-1BB domain.

In an example, the first intracellular domain is a CD3ζ domain, CD28 domain or 4-1BB domain disclosed in the sequence listing table herein.

(A): In an example, the CAL is an engineered single polypeptide comprising (in N- to C-terminal direction) a human CD3 extracellular domain; an optional hinge (eg, a human CD8a hinge); a transmembrane domain (eg, a human CD8a or CD28 transmembrane domain); and a human CD3ζ domain. In an example, the CAL is a complex of two or more of said polypeptides. Optionally, the CAL comprises a further intracellular signalling domain (i) between the transmembrane and CD3ζ domains. Optionally, the CAL comprises a further intracellular signalling domain, wherein the CD3ζ domain is between the further signaling domain and the transmembrane domain. In an example, the further signalling domain is a human CD27 domain, CD28 domain, ICOS domain, OX40 domain, CD40 domain, 4-1BB domain, a FcεRlγ domain, CD64 domain or CD16 domain. In an alternative, instead of a single polypeptide, the CAL comprises an engineered complex of at least 2 polypeptides comprising said domains. In an alternative, where a CAR is used in the invention, the CAR is identical to such a CAL with the exception that the CAR has an antigen binding site in place of the CD3 extracellular domain.

(B): In an example, the CAL is an engineered single polypeptide comprising (in N- to C-terminal direction) a human CD16 (eg, CD16A) extracellular domain; an optional hinge (eg, a human CD8α hinge); a transmembrane domain (eg, a human CD8α transmembrane domain); and a human CD3ζ domain. In an example, the CAL is a complex of two or more of said polypeptides. Optionally, the CAL comprises a further intracellular signalling domain (i) between the transmembrane and CD3ζ domains. Optionally, the CAL comprises a further intracellular signalling domain, wherein the CD3ζ domain is between the further signaling domain and the transmembrane domain. In an example, the further signalling domain is a human CD27 domain, CD28 domain, ICOS domain, OX40 domain, CD40 domain, 4-1BB domain, a FcεRlγ domain, CD64 domain or CD3 domain. In an alternative, instead of a single polypeptide, the CAL comprises an engineered complex of at least 2 polypeptides comprising said domains. In an alternative, where a CAR is used in the invention, the CAR is identical to such a CAL with the exception that the CAR has an antigen binding site in place of the CD16 extracellular domain.

(C): In an embodiment, the CAL is a complex of two or more of polypeptides, a first said CAL polypeptide being according to (A) and a second said polypeptide being a CAL polypeptide according to (B). In another embodiment, the CAR is a complex of two or more of polypeptides, a first said CAL polypeptide being according to (A) and a second said polypeptide being a CAR polypeptide according to (B).

In an example, the CAL-cell does not express said second antigen or a naturally-occurring variant thereof from an endogenous nucleotide sequence of the cell. For example, the endogenous sequence has been inactivated in the cell, eg, by being wholly or partially knocked out, or by mutation. For example, the mutation is a product of CRISPR/Cas-mediated genomic modification. By removing the possibility of expressing endogenous second antigen (eg, CD3 extracellular domain), all engagement of the second antigen is directed to triggering the CAL (and not to any endogenous receptor complex, such as an endogenous TCR-CD3 complex) which might otherwise compete for binding to the bridging agent.

In one embodiment, the immune cells (eg CAR- or CAL-cells) of the invention are administered in conjunction with an immunosuppressant agent. Any immunosuppressant agent known in the art may be used. For example, the immunosuppressant agent may be Cyclosporine, Azathioprine, Rapamycin, Mycophenolate mofetil, Mycophenolic acid, Prednisone, Sirolimus, Basiliximab, or Daclizumab, or any combination thereof.

Additional or alternative immunosuppressants that may be used include, but are not limited to, ORTHOCLONE OKT™ 3 (muromonab-CD3), SANDIMMUNE™, NEORAL™, SANGDYA™ (cyclosporine), PROGRAF™ (FK506, tacrolimus), CELLCEPT™ (mycophenolate motefil, of which the active metabolite is mycophenolic acid), IMURAN™ (azathioprine), glucorticosteroids, adrenocortical steroids such as DELTASONE™ (prednisone) and HYDELTRASOL™ (prednisolone), FOLEX™ and MEXATE™ (methotrxate), OXSORALEN-ULTRA™ (methoxsalen), RITUXAN™ (rituximab), and RAPAMUNE™ (sirolimus).

The immune cells of the invention can be administered to the patient before, after, or concomitant with the immunosuppressant agent. For example, the cells of the invention can be administered after the immunosuppressant agent is administered to the patient or the cells of the invention can be administered before the immunosuppressant agent is administered to the patient. Alternatively, or in addition, the cells of the invention are administered at the same time the immunosuppressant agent is administered to the patient.

The immune cells of the invention and/or the immunosuppressant agent can be administered to the patient after transplantation of an organ or tissue. Alternatively, or in addition, the immune cells of the invention and/or the immunosuppressant agent can be administered to the patient before transplantation. The immune cells of the invention and/or the immunosuppressant agent also can be administered to the patient during transplantation surgery.

In some embodiments, the method of the invention of administering immune cells to the patient is carried out once immunosuppressive therapy has been initiated. In some embodiments, the method is carried out more than once, e.g., to monitor the transplant recipient over time, and, if applicable, in different immunosuppressive therapy regimes. In some embodiments, immunosuppressive therapy is reduced if the transplant recipient is predicted to be tolerant of the transplant. In some embodiments, no immunosuppressive therapy is prescribed, e.g., immunosuppressive therapy is ceased, if the transplant recipient is predicted to be tolerant of the transplant. If the transplant recipient demonstrates a non-tolerant biomarker signature, immunosuppressive therapy can be restored to or continued at a standard level.

The organ or tissue transplant may be a heart, heart valve, lung, kidney, liver, pancreas, intestine, skin, blood vessels, bone marrow, stem cells, bone, or, islet cells.

The immune cells of the present invention may be administered either alone, or as a pharmaceutical composition in combination with diluents and/or with other components such as IL-2 or other cytokines or cell populations.

Tumour antigens (TAA) are proteins that are produced by tumour cells that elicit an immune response, particularly T-cell mediated immune responses. The selection of the first antigen binding specificity of the bridging agent of the invention will depend on the particular type of cancer to be treated. Tumour antigens are well known in the art and include, for example, a glioma-associated antigen, carcinoembryonic antigen (CEA), β-human chorionic gonadotropin, alphafetoprotein (AFP), lectin-reactive AFP, thyroglobulm, RAGE-1, MN-CA IX, human telomerase reverse transcriptase, RU1, RU2 (AS), intestinal carboxyi esterase, mut hsp70-2, M-CSF, prostase, prostate-specific antigen (PSA), PAP, NY-ESO-1, LAGE-Ia, p53, prostein, PSMA, Her2/neu, survivin and telomerase, prostate-carcinoma tumour antigen-1 (PCTA-1), MAGE, ELF2M, neutrophil elastase, ephrinB2, CD22, insulin growth factor (IGF)-I, IGF-II, IGF-I receptor and mesothelin. The first antigen (1^{st} to 4^{th} configuration of the invention) or fourth binding moiety (5^{th} or 6^{th} configuration) can be any of these TAAs or can be an antigenic sequence of any of these TAAs.

In one embodiment, the tumour antigen comprises one or more antigenic cancer epitopes associated with a malignant tumour. Malignant tumours express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu ErbB-2. Yet another group of target antigens are onco-foetal antigens such as carcinoembryonic antigen (CEA). In B-cell lymphoma the tumour-specific idiotype immunoglobulin constitutes a truly tumour-specific immunoglobulin antigen that is unique to the individual tumour. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success. The first antigen or fourth binding moiety can be any of these TAAs or can be an antigenic sequence of any of these TAAs.

Non-limiting examples of TAA antigens include the following: Differentiation antigens such as MART-I/MelanA (MART-1), g I OO (Pmel 17), tyrosinase, TRP-1, TRP-2 and tumour-specific multilineage antigens such as MAGE-1, MAGE-3, BAGE, GAGE-1, GAGE-2, pi 5; overexpressed embryonic antigens such as CEA; overexpressed oncogenes and mutated tumour-suppressor genes such as p53, Ras, HER-2/neu; unique tumour antigens resulting from chromosomal translocations; such as BCR-ABL, E2A-PRL, H4-RET, 1GH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl 85erbB2, p I 80erbB-3, c-met, nm-23H I, PSA, TAG-72, CA 19-9, CA 72-4, CAM 17.1, NuMa, K-ras, beta-Catenin, CDK4, Mum-1, p 15, p 16, 43-9F, 5T4(791Tgp72} alpha-fetoprotem, beta-HCG, BCA225, BTAA, CA 125, CA 15-3\CA 27.29\BCAA, CA 195, CA 242, CA-50, CAM43, CD68\ 1, CO-029, FGF-5, G250, Ga733VEpCAM, HTgp-175, M344, MA-50, MG7-Ag, MOV 18, NB/70K, NY-CO-1, RCAS 1, SDCCAG16, TA-90\Mac-2 binding proteiiAcyclophilin C-associated protein, TAAL6, TAG72, TLP, and TPS.

In one embodiment, the first antigen or fourth binding moiety is human CD 19 and the first antigen binding site or first binding moiety of the bridging agent is an anti-CD 19 scFV, optionally wherein the anti-CD19 scFV is encoded by SEQ ID: 14 disclosed in WO2012079000A1. In one embodiment, the anti-CD 19 scFV comprises the amino acid sequence of SEQ ID NO: 20. The sequences in this paragraph appear in WO2012079000A1 and are explicitly incorporated herein for use in the present invention in a bridging agent and for possible inclusion in one or more claims herein.

In one embodiment, the transmembrane domain that naturally is associated with one of the domains in the CAR or CAL is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CDS, CD9, CD16, CD22, CD33, CD37, CD64, CD80, CD86, CD 134, CD137 or CD 154. Alternatively the transmembrane domain may be synthetic, in which case it will comprise predominantly hydrophobic residues such as leucine and valine. Optionally, a triplet of phenylalanine, tryptophan and valine will be found at each end of a synthetic transmembrane domain.

Optionally, a short oligo- or polypeptide linker, preferably between 2 and 10 amino acids in length forms a linkage between the transmembrane domain and the intracellular part of the transmembrane protein (eg, CAL or CAR). A glycine-serine doublet provides a particularly suitable linker (eg, a (G₄S)ₙ linker as disclosed herein).

Optionally, the transmembrane domain is the CD8 transmembrane domain encoded by the nucleic acid sequence of SEQ ID NO: 16. In one embodiment, the CD8 transmembrane domain comprises the amino acid sequence of SEQ ID NO: 22. The sequences in this paragraph appear in WO2012079000A1 and are explicitly incorporated herein for use in the present invention in a bridging agent and for possible inclusion in one or more claims herein.

In some instances, the transmembrane domain comprises the CD8 hinge domain encoded by the nucleic acid sequence of SEQ ID NO: 15. In one embodiment, the CD8 hinge domain comprises the amino acid sequence of SEQ ID NO: 21. The sequences in this paragraph appear in WO2012079000A1 and are explicitly incorporated herein for use in the present invention in a bridging agent and for possible inclusion in one or more claims herein.

The intracellular part or otherwise the intracellular signaling domain(s) of the transmembrane protein of the invention is responsible for activation of at least one of the normal effector functions of the immune cell that expresses the transmembrane protein (eg, CAL or CAR). The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term “signaling domain” is thus meant to include any truncated portion of the intracellular signaling domain sufficient to transduce the effector function signal. Examples of intracellular signaling domains for use in the transmembrane protein of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling domain) and those that act in an antigen-independent manner to provide a secondary or co-stimulatory signal (secondary cytoplasmic signaling domain). Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

In an example, the first signalling domain is a primary cytoplasmic signaling domain (eg, CD3 domain). In an example, the first signalling domain is a secondary cytoplasmic signaling domain (eg, CD28 or 4-1BB domain).

In an example, the first signalling domain comprises one or more ITAMs.

Examples of suitable ITAM containing primary cytoplasmic signaling domains that are of particular use in the invention include those derived from TCR zeta, FcR gamma, FcR beta, CD3 gamma, CD3 delta, CD3 epsilon, CDS, CD22, CD79a, CD79b, and CD66d. It is particularly preferred that cytoplasmic signaling molecule in the transmembrane protein of the invention comprises a cytoplasmic signaling sequence derived from CD3 zeta.

The intracellular part optionally comprises (eg, as the first signalling domain or a further intracellular domain) a domain of a costimulatory molecule. A costimulatory molecule is a cell surface molecule other than an antigen receptor or their ligands that is required for an efficient response of lymphocytes (eg, T- or NK cells) to an antigen. Examples of such molecules include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. Thus, these and other costimulatory elements are within the scope of the invention for use in the intracellular part of the transmembrane protein.

The intracellular moiety domains may be linked together by one or more linkers, eg, a (G₄S), linker as disclosed herein.

In one embodiment, the intracellular part comprises the signaling domain of CD3-zeta and the signaling domain of CD28. In another embodiment, the intracellular part comprises the signaling domain of CD3-zeta and the signaling domain of 4-1BB. In yet another embodiment, the intracellular part comprises the signaling domain of CD3-zeta and the signaling domain of CD28 and 4-1BB.

In one embodiment, the intracellular v comprises the signaling domain of 4-1BB and the signaling domain of CD3-zeta, wherein the signaling domain of 4-1BB is encoded by the nucleic acid sequence set forth in SEQ ID NO: 17 and the signaling domain of CD3-zeta is encoded by the nucleic acid sequence set forth in SEQ ID NO: 18. The sequences in this paragraph appear in WO2012079000A1 and are explicitly incorporated herein for use in the present invention in a bridging agent and for possible inclusion in one or more claims herein.

In one embodiment, the intracellular part comprises the signaling domain of 4-1BB and the signaling domain of CD3-zeta, wherein the signaling domain of 4-1BB comprises the amino acid sequence of SEQ ID NO: 23 and the signaling domain of CD3-zeta comprises the amino acid sequence of SEQ ID NO: 24. The sequences in this paragraph appear in WO2012079000A1 and are explicitly incorporated herein for use in the present invention in a bridging agent and for possible inclusion in one or more claims herein.

In one embodiment, the intracellular part comprises the signaling domain of 4-1BB and the signaling domain of CD3-zeta, wherein the signaling domain of 4-1BB comprises the amino acid sequence set forth in SEQ ID NO: 23 and the signaling domain of CD3-zeta comprises the amino acid sequence set forth in SEQ ID NO: 24. The sequences in this paragraph appear in WO2012079000A1 and are explicitly incorporated herein for use in the present invention in a bridging agent and for possible inclusion in one or more claims herein.

The invention provides a nucleic acid vector comprising an expressible nucleotide sequence encoding a transmembrane protein, eg, CAR or CAL of the invention. The invention provides a first nucleic acid vector comprising an expressible nucleotide sequence encoding the transmembrane protein of the invention and a second nucleic acid vector comprising an expressible nucleotide sequence encoding the bridging agent of the invention. In an embodiment, the invention (eg, any method disclosed herein for treating or reducing the risk of a disease or condition in a patient, such as a human) comprises administering the transmembrane protein-encoding vector to a patient, whereby the vector is introduced into one or more first cells of the patient for expression of the transmembrane protein. In an example, the transmembrane protein is expressed in progeny cells, wherein the cells are progeny of the first cells. In an example, the first cells are stem cells (eg, bone marrow cells, haematopoietic stem cells and/or T memory cells) of the patient. As discussed below, the art already includes suitable vectors and techniques for performing this embodiment, for example the vector is a lentivirus, adenovirus or retrovirus. In an example, the transmembrane protein-encoding DNA is genomically incorporated in the first cells. This avoids the need to harvest ancestor cells for ex vivo engineering to encode the transmembrane protein, followed by infusion into a patient. Instead, vector administration is the only step required and compatibility of the resultant progeny cells is maximised as these are based only on first cells of the patient, without risk of change caused by ex vivo manipulation. Furthermore, the patient's own system (optionally stimulated with an agent such as IL-2, which up-regulates immune cell expansion) can be administered to expand the progeny cell population. The bridging agent can, for example, be produced ex vivo and administered to the patient after the patient has produced the progeny cells, whereby the titratable advantages of the method of the invention can be realised.

In an example, the vector is a gene therapy vector for introduction into a human cell, eg, a human T-cell, NK cell, TIL, stem cell, bone marrow cell or progenitor cell thereof. The invention also provides such a cell comprising the transmembrane protein-encoding nucleotide sequence (eg, DNA) or vector. The invention also comprises a retrovirus, adenovirus or lentivirus comprising an expressible nucleotide sequence encoding a transmembrane protein (eg, CAL) of the invention. The sequences are expressible when comprised by an immune cell of the invention, eg, expressible in a human T-cell, NK cell or TIL.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukaemia viruses in that they can transduce non-proliferating cells. They also have the added advantage of low immunogenicity.

The constructs and vectors of the present invention may also be used for nucleic acid immunisation and gene therapy, using standard gene delivery protocols. Methods for gene delivery are known in the art. See, e.g., U.S. Pat. Nos. 5,399,346, 5,580,859, 5,589,466, incorporated by reference herein in their entireties.

The invention provides an mRNA encoding a transmembrane protein, eg, CAL or CAR of the invention. The skilled addressee will be aware of techniques to deliver mRNAs into organisms, such as humans, for expression of the encoded proteins in vivo. In an example, the invention comprises introducing the mRNA into a first immune cell or first immune cell progenitor (eg, a human T-cell, NK cell of TIL or a haematopoietic stem cell or T-memory stem cell) for expression of the transmembrane protein in the cell or a progeny thereof. Optionally the cell or progeny product is administered to a patient (eg, a human) in a method of treatment or prevention of a disease or condition as described herein. In an alternative, the first cell is comprised by a patient, eg, a human, when the mRNA is introduced into the cell.

Sources of T-cells and other immune cells are disclosed in WO2012079000A1, as well as methods of generating, activating and expanding these. These disclosures are referred to for possible use in working the present invention.

### Cancers

Cancers that may be treated include tumours that are not vascularized, or not substantially vascularized, as well as vascularized tumours. The cancers may comprise non-solid tumours (such as haematological tumours, for example, leukaemias and lymphomas) or may comprise solid tumours. Types of cancers to be treated with the CALs of the invention include, but are not limited to, carcinoma, blastoma, and sarcoma, and certain leukaemia or lymphoid malignancies, benign and malignant tumours, and malignancies e.g., sarcomas, carcinomas, and melanomas. Adult tumours/cancers and paediatric tumours/cancers are also included.

Hematologic cancers are cancers of the blood or bone marrow. Examples of haematological (or haematogenous) cancers include leukaemias, including acute leukaemias (such as acute lymphocytic leukaemia, acute myelocytic leukaemia, acute myelogenous leukaemia and myeloblasts, promyeiocytic, myelomonocytic, monocytic and erythroleukaemia), chronic leukaemias (such as chronic myelocytic (granulocytic) leukaemia, chronic myelogenous leukaemia, and chronic lymphocytic leukaemia), polycythemia vera, lymphoma, Hodgkin's disease, non-Hodgkin's lymphoma (indolent and high grade forms), multiple myeloma, Waldenstrom's macroglobulinemia, heavy chain disease, myeiodysplastic syndrome, hairy cell leukaemia and myelodysplasia.

Solid tumours are abnormal masses of tissue that usually do not contain cysts or liquid areas. Solid tumours can be benign or malignant. Different types of solid tumours are named for the type of cells that form them (such as sarcomas, carcinomas, and lymphomas). Examples of solid tumours, such as sarcomas and carcinomas, include fibrosarcoma, myxosarcoma, liposarcoma, chondrosarcoma, osteosarcoma, and other sarcomas, synovioma, mesothelioma, Ewing's tumour, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous eel! carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumour, cervical cancer, testicular tumour, seminoma, bladder carcinoma, melanoma, and CNS tumours (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pineaioma, hemangioblastoma, acoustic neuroma, oligodendroglioma, menangioma, neuroblastoma, retinoblastoma and brain metastases).

In one embodiment, the first binding moiety or the first antigen binding site of the bridging agent of the invention is designed to treat a particular cancer. For example, it specifically binds to CD19 can be used to treat cancers and disorders, eg, pre-B ALL (paediatric indication), adult ALL, mantle cell lymphoma, diffuse large B-cell lymphoma or for salvage post allogenic bone marrow transplantation. In another embodiment, the first moiety or first binding site specifically binds CD22 to treat diffuse large B-cell lymphoma.

In one embodiment, cancers and disorders include but are not limited to pre-B ALL (paediatric indication), adult ALL, mantle cell lymphoma, diffuse large B-cell lymphoma, salvage post allogenic bone marrow transplantation, and the like can be treated using a combination of bridging agents (or binding moieties or sites comprised by a single agent) that target two or three of: CD19, CD20, CD22, and ROR1 (eg, CD19 and one of the other targets).

In an example, the cell of the invention comprises first and second transmembrane proteins (eg, CALs or CARs) that are different, eg CALs that differ in their second antigens (and optionally otherwise are the same). Similarly, the invention provides a mixture of immune cells (eg, a mixture of CAL-cells) of the invention, eg comprised by a transplant of the invention, wherein the mixture comprises cells comprising different transmembrane proteins (eg, different CALs differing in their second antigen). In an example, the cell of the invention comprises first and second bridging agents that are different, eg differ in their first moiety/first antigen binding site specificities (and optionally otherwise are the same, eg, comprise the same second moiety/second antigen binding site). This may be useful for reducing resistance to treatment by cancers, for example, or more effectively targeting cell populations such as cancer cells that surface express a plurality of target antigens.

In one embodiment, the bridging agent's first moiety/first antigen binding site specifically binds to mesothelin to treat or prevent mesothelioma, pancreatic cancer or ovarian cancer.

In one embodiment, the bridging agent's first moiety/first antigen binding site specifically binds to CD33/IL3Ra to treat or prevent acute myelogenous leukaemia.

In one embodiment, the bridging agent's first moiety/first antigen binding site specifically binds to c-Met to treat or prevent triple negative breast cancer or non-small cell lung cancer.

In one embodiment, the bridging agent's first moiety/first antigen binding site specifically binds to PSMA to treat or prevent prostate cancer.

In one embodiment, the bridging agent's first moiety/first antigen binding site specifically binds to Glycolipid F77 to treat or prevent prostate cancer.

In one embodiment, the bridging agent's first moiety/first antigen binding site specifically binds to EGFRvIII to treat or prevent gliobastoma.

In one embodiment, the bridging agent's first moiety/first antigen binding binding site specifically binds to GD-2 to treat or prevent neuroblastoma or melanoma.

In one embodiment, the bridging agent's first moiety/first antigen binding site specifically binds to NY-ESO-1 TCR to treat myeloma, sarcoma or melanoma.

In one embodiment, the bridging agent's first moiety/first antigen binding site specifically binds to MAGE A3 TCR to treat myeloma, sarcoma and melanoma.

In one example, said treatment using the method reduces progression of the disease or condition or a symptom thereof. In one example, said treatment using the method reduces incidence of the disease or condition or symptom thereof, eg, for at least 1, 2, 3, 4, or 5 years.

In an example, the method of the invention is performed ex vivo to produce a transplant wherein target cells have been killed or reduced in number, wherein the transplant is for administration (eg, infusion) to a patient (eg, human) for treating or reducing the risk of a disease or condition in the human.

In an example, the method is in vitro. In another example, the method is in vivo in a mammal, eg, a human, man or woman, or male child or female child, or a human infant (eg, no more than 1, 2, 3 or 4 years of age). In an example, the patient is an adult human or a paediatric human patient.

### Specific Embodiments

The invention provides specific embodiments in the following numbered paragraphs:—
1. A method of targeting an immune cell (eg, a T-cell, NK cell or TIL) to a target cell, the method comprising
   A. Providing a bridging agent, wherein the agent is a multi-specific antigen binding fragment comprising
      i. a first antigen binding site that specifically binds a first target antigen; and
      ii. a second antigen binding site that specifically binds a second target antigen;
   B. Providing a chimaeric antigen ligand (CAL)-immune cell, wherein the immune cell comprises a transmembrane ligand, the ligand comprising an engineered combination of
      iii. an extracellular moiety comprising the second antigen, wherein the second antigen is linked to a transmembrane domain; and
      iv. an intracellular moiety comprising a first signaling domain for intracellular signaling when the agent binds to the second antigen;
   C. Combining the CAL-immune cell and bridging agent with the target cell, the target cell comprising said first target antigen, wherein the first antigen is an extracellular antigen,
      v. whereby the bridging agent binds to the first and second antigens to target the immune cell to the target cell,
      vi. thereby triggering intracellular signaling in the immune cell to regulate immune cell activity.

The CAL is engineered, ie, comprises a non-naturally-occurring combination of moieties and domains. For example, the ligand comprises a single polypeptide (ie, has only one such polypeptide) that comprises CD3 extracellular domain (second antigen), CD28 or 4-1BB domain (first signaling domain) and a CD3 zeta domain. In this case, the ligand comprises an engineered domain combination, since the CD3 extracellular domain and the CD28 or 4-1BB domains do not naturally occur in the same receptor (eg, not in a natural CD3 receptor complex). Thus, in an example the second antigen and the first signaling domain are not naturally comprised by a receptor of the cell or not naturally comprised by humans or the human that is the subject of the method of the invention. For example, the immune cell does not comprise endogenous nucleotide sequence(s) encoding a receptor comprising said combination.

By the term “specifically binds,” as used herein with respect to an antibody or binding site, is meant an antibody or binding site which recognises a specific antigen with a binding affinity of 1 mM or less as determined by SPR.

Target binding ability, specificity and affinity (KD (also termed Kd), K_{off} and/or Kₒₙ) can be determined by any routine method in the art, eg, by surface plasmon resonance (SPR). The term “KD”, as used herein, is intended to refer to the equilibrium dissociation constant of a particular binding site/ligand, receptor/ligand or antibody/antigen interaction.

In one embodiment, the surface plasmon resonance (SPR) is carried out at 25° C. In another embodiment, the SPR is carried out at 37° C.

In one embodiment, the SPR is carried out at physiological pH, such as about pH7 or at pH7.6 (eg, using Hepes buffered saline at pH7.6 (also referred to as HBS-EP)).

In one embodiment, the SPR is carried out at a physiological salt level, eg, 150 mM NaCl.

In one embodiment, the SPR is carried out at a detergent level of no greater than 0.05% by volume, eg, in the presence of P20 (polysorbate 20; eg, Tween-20™) at 0.05% and EDTA at 3 mM.

In one example, the SPR is carried out at 25° C. or 37° C. in a buffer at pH7.6, 150 mM NaCl, 0.05% detergent (eg, P20) and 3 mM EDTA. The buffer can contain 10 mM Hepes. In one example, the SPR is carried out at 25° C. or 37° C. in HBS-EP. HBS-EP is available from Teknova Inc (California; catalogue number H8022).

In an example, the affinity (eg, of an agent comprising a VH/VL binding site) is determined using SPR by using any standard SPR apparatus, such as by Biacore™ or using the ProteOn XPR36™ (Bio-Rad®). The binding data can be fitted to 1:1 model inherent using standard techniques, eg, using a model inherent to the ProteOn XPR36™ analysis software.

Between the second antigen (which can be a domain or peptide) and the transmembrane domain of the CAL, or between the first signalling domain and the transmembrane domain of the CAL, there is optionally a spacer (domain or peptide). As used herein, the term “spacer” generally means any oligo- or polypeptide that functions to link the transmembrane domain to, either the second antigen or, the signaling domain in the CAL. A spacer may comprise up to 300 amino acids, preferably from 10 to 100 amino acids and most preferably from 25 to 50 amino acids.

Optionally, each binding site of the bridging agent comprises an antibody binding site, eg, a VH/VL binding site for an antigen.

Said signaling can up- or down-regulate immune cell activity, eg, cytotoxicity or cell proliferation.

### 5^{th} & 6^{th} Configuration Examples

In an alternative, paragraph 1 provides the following alternative clauses I to VII. Hence, features of paragraphs 2 onwards below are combinable with any of clauses I to VII. Reference to “paragraph 1” below can thus in the alternative be read as any one of clauses I to VII; “first antigen binding site” in any one of paragraphs 2 onwards can be read as “first binding moiety” as recited in any of any of clauses I to VII; “second antigen binding site” in any one of paragraphs 2 onwards can be read as “second binding moiety” as recited in any of any of clauses I to VII; “first antigen” in any one of paragraphs 2 onwards can be read as “fourth binding moiety” as recited in any of any of clauses I to VII; “second antigen” in any one of paragraphs 2 onwards can be read as “third binding moiety” as recited in any of any of clauses I to VII; and CAL in any one of paragraphs 2 onwards can be read as the “transmembrane protein”, “CAR” or “CAL” of any of clauses I to VII.

I. A method of targeting an immune cell to a target cell, the method comprising

A. Providing a bridging agent, wherein the agent is a multi-specific binding fragment comprising
vii. a first binding moiety; and
viii. a second binding moiety;

B. Providing an immune cell, wherein the immune cell expresses a transmembrane protein comprising an engineered combination of
ix. ‘an extracellular part comprising a third binding moiety that is linked to a transmembrane domain; wherein the second and third moieties form a specific binding pair (SBP1) wherein one moiety specifically binds to the other moiety; and
x. an intracellular part comprising a first signaling domain for intracellular signaling when the second and third moieties bind together;

C. Combining the immune cell and bridging agent with the target cell, the target cell comprising a fourth binding moiety, wherein the fourth moiety is extracellular,
xi. whereby the first and fourth moieties form a specific binding pair (SBP2) wherein one moiety specifically binds to the other moiety to target the immune cell to the target cell,
xii. wherein the second and third moieties bind together thereby triggering intracellular signaling in the immune cell to regulate immune cell activity; and
xiii. wherein the molecular weight of the bridging agent is no more than 125 kDa.

II. The method of clause I, wherein the first and second binding moieties are ligand binding sites.

III. The method of clause II, wherein the first and second binding moieties are first and second antigen binding sites respectively (eg, VH/VL antigen binding sites).

It is well known how an antibody VH domain pairs with an antibody VL domain to form a VH/VL binding site that specifically binds an antigen.

IV. The method of any one of clause I to III, wherein each of SBP1 and SB2 is selected from the group consisting of:—

(a) an antigen and an antigen binding site (eg, an antigen and a VH/VL antigen binding site; or a superantigen and an antibody variable domain or constant domain); and

(b) a receptor and a ligand.

Examples of superantigens are protein A (eg, the ligand binding domain of protein A from S aureus), protein G and protein L ((eg, the ligand binding domain of protein L from Peptostreptococcus magnus) or a ligand binding domain of gp120.
In an example SB1 and/or SB2 each is a growth factor domain-growth factor receptor pair; or a hormone domain-hormone receptor pair. The receptor comprises a binding site for the cognate ligand, but otherwise need not be the complete receptor, ie, can be a receptor fragment.

V. The method of any one of clause I to IV, wherein the transmembrane protein is a chimaeric antigen ligand (CAL), wherein the third moiety is an antigen and the second moiety is an antigen binding site (eg, an scFv); and optionally the first moiety is an antigen binding site (eg, an scFv) and the fourth moiety is an antigen.

VI. The method of any one of clause I to IV, wherein the transmembrane protein is a chimaeric antigen receptor (CAR), wherein the third moiety is an antigen binding site and the second moiety is an antigen; and optionally the first moiety is an antigen binding site (eg, an scFv) and the fourth moiety is an antigen.

VII. The method of any one of clause I to VI, wherein the molecular weight of the bridging agent is no more than 115 kDa, eg, from 55 to 115 kDa or from 60 to 100 kDa.

The following features are applicable to all configurations.

In an example, the binding agent is a ligand trap whose molecular weight is no more than 125, 120, 115, 110, 100 or 50 kDa. In an example, the first binding moiety of the trap comprises a ligand binding site, eg, a binding site of a ligand receptor and the second binding site comprises an antibody Fc region (eg, a human IgG1 Fc region), wherein the third binding moiety comprises a binding site of an Fc receptor (eg, CD16, CD16A or CD16B) and the fourth binding moiety is comprised by said ligand. Such binding agents of the invention beneficially have shorter half-lives than antibodies, as described above. In an example, the ligand is human IL-1A, IL-1β, IL-1RN, IL-6, BLys, APRIL, activin A, TNF alpha, a BMP, BMP2, BMP7, BMP9, BMP10, GDF8, GDF11, RANKL, TRAIL, VEGFA, VEGFB or PGF. In an example, the first binding moiety comprises an IL-1R domain, a gp130 domain, an ActRIIA domain, an ActRIIB domain, an Alk1 domain, an OPG domain and a VEGFR1 and/or VEGFR2 domain. In an example, the first binding moiety comprises a ligand binding siste of human IL-1R, gp130, ActRIIA domain, ActRIIB, Alk1, OPG, VEGFR1, or VEGFR2.

In an example, the agent is selected from the group consisting of aflibercept, Zaltrap™, or Eylea™ (and the fourth binding moiety is VEGFA, VEGFB or PGF), ranibizumab or Lucentis™ (and the fourth binding moiety is VEGFA, VEGFB or PGF), etanercept or Enbrel™ (and the fourth binding moiety is TNF alpha), certolizumab (ie, the Fab of certolizumab pegol or Cimzia™ excluding PEG) (and the fourth binding moiety is TNF alpha), atacicept (and the fourth binding moiety is BLys), rilonacept or Arcalyst™ (and the fourth binding moiety is IL-1); or the first binding moiety of the bridging agent of the invention comprises a ligand binding site of an agent selected from said group.

In an example, each binding moiety is human or derived from a human moiety, eg, a human ligand or human binding site.

2. The method of paragraph 1, wherein the bridging agent has a human serum half-life that is less than the human serum half-life of IgG.

Optionally, the bridging agent has a human serum half-life that is less than the human serum half-life of IgA. Optionally, the bridging agent has a human serum half-life that is less than the human serum half-life of IgM. Optionally, the bridging agent has a human serum half-life that is less than the human serum half-life of IgD. Optionally, the bridging agent has a human serum half-life that is less than the human serum half-life of IgE.

3. The method of paragraph 2, wherein the bridging agent has a human serum half-life of no more than 15, 10 or 5 days.

The skilled addressee will know how to routinely determine such half-lives. Serum half-lives for many of the prior art fragments, including fragments disclosed herein, are known in the art.

4. The method of paragraph 3, wherein the half-life is less than 1 day.
5. The method of any preceding paragraph, wherein the first binding site is a VH/VL binding site.
6. The method of any preceding paragraph, wherein the second binding site is a VH/VL binding site.
7. The method of any preceding paragraph, wherein the binding affinity (KD) of the first binding site for the first antigen is at least 5-, 10- or 20-fold lower than the affinity of the second binding site for the second antigen.
Thus binding to the first antigen is stronger than for the second antigen. This can be useful to control the switching activity of the bridging agent.
For example, blinatumomab targets malignant B cells highly specifically (affinity of 1.6×10⁻⁹M) via CD19, a marker solely expressed by B cells. Also blinatumomab recruits and activates T cells via a lower affinity interaction with CD3 (8.7×10⁻⁸M). In an example, the bridging agent is blinatumomab or comprises blinatumomab. The sequence of blinatumomab is shown in SEQ ID NO: 19 below. In an embodiment, therefore, the agent comprises SEQ ID NO: 19.
   8. The method of any preceding paragraph, wherein the first binding site has a binding affinity (KD) for the first antigen of 10 nM or less as determined by surface plasmon resonance (SPR); and the second binding site has a binding affinity (KD) for the second antigen of 50 nM or more (eg, up to 1 mM) as determined by SPR.
This is useful to produce preferential binding to the target cell than to the immune cell, to aid moderation of immune cell activity in vivo.

The binding affinity of natural TCR-peptide/MHC interactions is around KD˜0.1-500 μM. In an example, the KD for binding of the second binding site to the second antigen (1^{st} to 4^{th} configurations)/second moiety to the third moiety (5^{th} or 6^{th} configurations) of the invention is less than 100 nM, eg, 50 nM≥KD<100 nM, eg, from 50 nM to 95, 90, 85 or 80 nM. Affinities lower than 100 nM are useful to promote preferential binding to the engineered transmembrane protein (eg, CAL or CAR) rather than natural TCR binding on the surface of immune cells of the invention. In an example, the immune cell is a CAL-T or CAR-T cell and the binding affinity of the bridging agent for the first antigen is higher than the affinity of the bridging agent for the second antigen/third moiety, wherein the affinity for the second antigen/third moiety is less than 100, 90 or 85 nM. Thus, when TCR and second antigen or third moiety are co-expressed on an immune cell of the invention, by choosing relative binding affinities in this way, the cell can be preferentially bound to the engineered transmembrane protein (eg, CAL or CAR) of the invention rather than via any endogenous TCR of the T-cell. This is useful to drive signalling via the transmembrane protein.

9. The method of any preceding paragraph, wherein the first binding site has a binding affinity (KD) for the first antigen of 2 nM or less as determined by SPR and the second binding site has a binding affinity (KD) for the second antigen of 60 nM or more (eg, up to 1 mM) as determined by SPR.
10. The method of any preceding paragraph, wherein the first binding site has a binding affinity (KD) for the first antigen of 100 nM or less as determined by surface plasmon resonance (SPR).
11. The method of any preceding paragraph, wherein the first binding site has a binding off-rate for the first antigen of K_{off}=10⁻³ sec or less as determined by SPR.
12. The method of any preceding paragraph, wherein the second binding site has a binding affinity (KD) for the second antigen of 100 nM or less as determined by surface plasmon resonance (SPR).
13. The method of any preceding paragraph, wherein the second binding site has a binding off-rate for the second antigen of K_{off}=10⁻³ sec⁻¹ or less as determined by SPR.
The invention includes the following optional embodiments in respect of the bridging agent:—

### First Binding Site Kinetics

(a) The first antigen binding site specifically binds human FA (FA=said first antigen) with a dissociation constant (KD) from (or from about) 0.1 to (or to about) 10000 nM, optionally from (or from about) 1 to (or to about) 6000 nM, as determined by surface plasmon resonance;
(b) The first antigen binding site specifically binds human FA with an off-rate constant (K_{d}) from (or from about) 1.5×10⁻⁴ to (or to about) 0.1 sec⁻¹, optionally from (or from about) 3×10⁻⁴ to (or to about) 0.1 sec⁻¹ as determined by surface plasmon resonance; and
(c) The first antigen binding site specifically binds human FA with an on-rate constant (Kₐ) from (or from about) 2×10⁶ to (or to about) 1×10⁴ M⁻¹ sec⁻¹, optionally from (or from about) 1×10⁶ to (or to about) 2×10⁴ M⁻¹ sec⁻¹ as determined by surface plasmon resonance;
optionally also:—
(d) The first antigen binding site specifically binds Cynomolgus monkey FA with a dissociation constant (KD) from (or from about) 0.1 to (or to about) 10000 nM, optionally from (or from about) 1 to (or to about) 6000 nM, as determined by surface plasmon resonance;
(e) The first antigen binding site specifically binds Cynomolgus monkey FA with an off-rate constant (K_{d}) from (or from about) 1.5×10⁻⁴ to (or to about) 0.1 sec⁻¹, optionally from (or from about) 3×10⁻⁴ to (or to about) 0.1 sec⁻¹ as determined by surface plasmon resonance; and
(f) The first antigen binding site specifically binds Cynomolgus monkey FA with an on-rate constant (Kₐ) from (or from about) 2×10⁶ to (or to about) 1×10⁴ M⁻¹ sec⁻¹, optionally from (or from about) 1×10⁶ to (or to about) 5×10³ M⁻¹ sec⁻¹ as determined by surface plasmon resonance.
Optionally, the first binding site has a KD according to (a) and (d), a K_{d} according to (b) and (e), and a Kₐ according to (c) and (f).

### Second Binding Site Kinetics

(a′) The second antigen binding site specifically binds human SA (SA=said second antigen) with a dissociation constant (KD) from (or from about) 0.1 to (or to about) 10000 nM, optionally from (or from about) 1 to (or to about) 6000 nM, as determined by surface plasmon resonance;
(b′) The second antigen binding site specifically binds human SA with an off-rate constant (K_{d}) from (or from about) 1.5×10⁻⁴ to (or to about) 0.1 sec⁻¹, optionally from (or from about) 3×10⁻⁴ to (or to about) 0.1 sec⁻¹ as determined by surface plasmon resonance; and
(c′) The second antigen binding site specifically binds human SA with an on-rate constant (Kₐ) from (or from about) 2×10⁶ to (or to about) 1×10⁴ M⁻¹ sec⁻¹, optionally from (or from about) 1×10⁶ to (or to about) 2×10⁴ M⁻¹ sec⁻¹ as determined by surface plasmon resonance;
optionally also:—
(d′) The second antigen binding site specifically binds Cynomolgus monkey SA with a dissociation constant (KD) from (or from about) 0.1 to (or to about) 10000 nM, optionally from (or from about) 1 to (or to about) 6000 nM, as determined by surface plasmon resonance;
(e′) The second antigen binding site specifically binds Cynomolgus monkey SA with an off-rate constant (K_{d}) from (or from about) 1.5×10⁻⁴ to (or to about) 0.1 sec⁻¹, optionally from (or from about) 3×10⁻⁴ to (or to about) 0.1 sec⁻¹ as determined by surface plasmon resonance; and
(f′) The second antigen binding site specifically binds Cynomolgus monkey SA with an on-rate constant (Kₐ) from (or from about) 2×10⁶ to (or to about) 1×10⁴ M⁻¹ sec⁻¹, optionally from (or from about) 1×10⁶ to (or to about) 5×10³ M⁻¹ sec⁻¹ as determined by surface plasmon resonance. Optionally, the second binding site has a KD according to (a′) and (d′), a K_{d} according to (b′) and (e′), and a Kₐ according to (c′) and (f′).
14. The method of any preceding paragraph, wherein each of the first and second antigen binding sites is selected from the group consisting of an scFv, Nanobody™, dAb, duocalin, DARpin, avimer, adnectin and fynomer.
15. The method of any preceding paragraph, wherein the size of the bridging agent is no more than 125 kDa.
In an example, the size is no more than 115, 110, 100, 90, 80, 70 or 60 kDa.
This is advantageous for providing a human serum half-life that has useful benefits of the present invention, as described above.
16. The method of any preceding paragraph, wherein the size of the bridging agent is no more than 80 kDa (eg, no more than 50 or 55 kDa).
17. The method of any preceding paragraph, wherein the bridging agent is or comprises a BiTE™ antibody, bispecific-scFv, trispecific-scFv, Tandab™, dAb nanobody (eg, dimer or trimer), dAb multimer (eg, dimer or trimer), diabody, tetrabody or DART™.

In an example, the bridging agent comprises one, two or more Fabs to provide the binding sites.

### Antigen Binding Sites

In an example, the or each antigen binding site (or ligand-binding moiety when according to the 5^{th} or 6^{th} configuration) is selected from the group consisting of an antibody variable domain (eg, a VL or a VH, an antibody single variable domain (domain antibody or dAb), a camelid VHH antibody single variable domain, a shark immunoglobulin single variable domain (NA V), a Nanobody™ or a camelised VH single variable domain); a T-cell receptor binding domain; an immunoglobulin superfamily domain; an agnathan variable lymphocyte receptor (J Immunol; 2010 August I; 185(3):1367-74; “Alternative adaptive immunity in jawless vertebrates; Herrin B R & Cooper M D.); a fibronectin domain (eg, an Adnectin™); an scFv; an (scFv)₂; an sc-diabody; an scFab; a centyrin and an antigen binding site derived from a scaffold selected from CTLA-4 (Evibody™); a lipocalin domain; Protein A such as Z-domain of Protein A (eg, an Affibody™ or SpA); an A-domain (eg, an Avimer™ or Maxibody™); a heat shock protein (such as and epitope binding domain derived from GroEI and GroES); a transferrin domain (eg, a trans-body); ankyrin repeat protein (eg, a DARPin™); peptide aptamer; C-type lectin domain (eg, Tetranectin™); human γ-crystallin or human ubiquitin (an affilin); a PDZ domain; scorpion toxin; and a kunitz type domain of a human protease inhibitor.

Further sources of antigen binding sites are variable domains and VH/VL pairs of antibodies disclosed in WO2007024715 at page 40, line 23 to page 43, line 23. This specific disclosure is incorporated herein by reference as though explicitly written herein to provide basis for epitope binding moieties for use in the present invention and for possible inclusion in claims herein. A “domain” is a folded protein structure which has tertiary structure independent of the rest of the protein. Generally, domains are responsible for discrete functional properties of proteins and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

The phrase “immunoglobulin single variable domain” refers to an antibody variable domain (VH, VHH, VL) that specifically binds an antigen or epitope independently of a different V region or domain. An immunoglobulin single variable domain can be present in a format (e.g., homo- or hetero-multimer) with other, different variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” which is capable of binding to an antigen as the term is used herein. An immunoglobulin single variable domain may be a human antibody variable domain, but also includes single antibody variable domains from other species such as rodent (for example, as disclosed in WO 00/29004), nurse shark and Camelid VHH immunoglobulin single variable domains. Camelid VHH are immunoglobulin single variable domain polypeptides that are derived from species including camel, llama, alpaca, dromedary, and guanaco, which produce heavy chain antibodies naturally devoid of light chains. Such VHH domains may be humanised according to standard techniques available in the art, and such domains are still considered to be “domain antibodies” according to the invention. As used herein “VH includes camelid VHH domains. NA V are another type of immunoglobulin single variable domain which were identified in cartilaginous fish including the nurse shark. These domains are also known as Novel Antigen Receptor variable region (commonly abbreviated to V(NAR) or NARV). For further details see Mol. Immunol. 44, 656-665 (2006) and US20050043519A. CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies. For further details see Journal of Immunological Methods 248 (1-2), 31-45 (2001). Lipocalins are a family of extracellular proteins which transport small hydrophobic molecules such as steroids, bilins, retinoids and lipids. They have a rigid β-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomisation of surface residues. For further details see Protein Eng. Des. Sel. 17, 455-462 (2004) and EP1641818A1. Avimers™ are multidomain proteins derived from the A-domain scaffold family. The native domains of approximately 35 amino acids adopt a defined disulphide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999). Designed Ankyrin Repeat Proteins (DARPins™) are derived from ankyrin which is a family of proteins that mediate attachment of integral membrane proteins to the cytoskeleton. A single ankyrin repeat is a 33 residue motif consisting of two a-helices and a β-turn. They can be engineered to bind different target antigens by randomising residues in the first a-helix and a β-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1. Fibronectin is a scaffold which can be engineered to bind to antigen. Adnectins™ consist of a backbone of the natural amino acid sequence of the 10th domain of the 15 repeating units of human fibronectin type III (FN3). Three loops at one end of the β-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges—examples of microproteins include KalataBI and conotoxin and knottins. The microproteins have a loop which can be engineered to include upto 25 amino acids without affecting the overall fold of the microprotein. For further details of engineered knottin domains, see WO2008098796. Other epitope binding moieties and domains include proteins which have been used as a scaffold to engineer different target antigen binding properties include human γ-crystallin and human ubiquitin (affilins), kunitz type domains of human protease inhibitors, PDZ-domains of the Ras-binding protein AF-6, scorpion toxins (charybdotoxin), C-type lectin domain (tetranectins) are reviewed in Chapter 7—Non-Antibody Scaffolds from Handbook of Therapeutic Antibodies (2007, edited by Stefan Dubel) and Protein Science 15:14-27 (2006). Antigen binding sites or ligand-binding moieties of the bridging agent of the present invention could be derived from any of these alternative protein domains.

18. The method of any preceding paragraph, wherein the CAL comprises a hinge region and/or a linker between the second antigen and the transmembrane domain.
19. The method of any preceding paragraph, wherein the target cell is a human cell.
20. The method of any preceding paragraph, wherein the bridging agent comprises a third antigen binding site.
21. The method of paragraph 20, wherein the third antigen is different from the first antigen.
22. The method of paragraph 20 or 21, wherein the third antigen is a tumour associated antigen (TAA), eg, a cell surface TAA comprised by the target cell.
23. The method of any preceding paragraph, wherein the first and/or third antigen is present more commonly on cancer cells than on normal cells.
24. The method of any preceding paragraph, wherein the target cell is a tumour cell and the signaling up-regulates cytotoxic activity (eg, ADCC) or proliferation of the immune cell.
25. The method of paragraph 24, wherein the signaling upregulates said cytotoxicity and the tumour cell is killed or tumour cell proliferation is down-regulated.
26. The method of any preceding paragraph, wherein the target cell is a leukaemic cell, lymphoma cell, adenocarcinoma cell or cancer stem cell.
27. The method of any preceding paragraph, wherein the first antigen is a tumour associated antigen (TAA).
28. The method of any preceding paragraph, wherein the first antigen is human CD19 (and optionally the target cell is a leukaemic or lymphoma cell), EpCAM (and optionally the target cell is a lung cancer cell, gastrointestinal cancer cell, an adenocarcinoma, cancer stem cell), CD20 (and optionally the target cell is a leukaemic cell), MCSP (and optionally the target cell is a melanoma cell), CEA, EGFR, EGFRvIII, sialyl Tn, CD133, CD33 (and optionally the target cell is a leukaemic cell, eg, AML cell), PMSA, WT1, CD22, L1CAM, ROR-1, MUC-16, CD30, CD47, CD52, gpA33, TAG-72, mucin, CIX, GD2, GD3, GM2, CD123, VEGFR, integrin, cMET, Her1, Her2, Her3, MAGE1, MAGE A3 TCR, NY-ESO-1, IGF1R, EPHA3, CD66e, EphA2, TRAILR1, TRAILR2, RANKL, FAP, Angiopoietin, mesothelin, Glycolipid F77 or tenascin.
29. The method of any preceding paragraph, wherein the first antigen binding site comprises the variable domains of an antibody selected from the group consisting of the CD19 binding site of blinatumomab or antibody HD37; EpCAM binding site of Catumaxomab; CD19 binding site of AFM11; CD20 binding site of Lymphomun; Her2 binding site of Ertumaxomab; CEA binding site of AMG211 (MEDI-565, MT111); PSMA binding site of Pasotuxizumab; EpCAM binding site of solitomab; VEGF or angiopoietin 2 binding site of RG7221 or RG7716; Her1 or Her3 binding site of RG7597; Her2 or Her3 binding site of MM111; IGF1R or Her3 binding site of MM141; CD123 binding site of MGD006; gpa33 binding site of MGD007; CEA binding site of TF2; CD30 binding site of AFM13; CD19 binding site of AFM11; and Her1 or cMet binding site of LY3164530.
30. The method of any preceding paragraph, wherein the bridging agent is blinatumomab or a CD3/CD19-binding derivative thereof (optionally wherein the target cell is an acute lymphoblastic leukaemia (ALL) B-cell); AMG211 or a CD3/CEA-binding derivative thereof (MEDI-565, MT111; optionally wherein the target cell is a Gastrointestinal cancer cell); Pasotuxizumab or a CD3/PMSA-binding derivative thereof (optionally wherein the target cell is a prostate cancer cell); solitomab or a CD3/EpCAM-binding derivative thereof (optionally wherein the target cell is a cancer cell); or AFM11 or a CD3/CD19-binding derivative thereof (and optionally wherein the target cell is an ALL cell or Non-Hodgkins Lymphoma cell).
31. The method of any one paragraphs 1 to 27, the first antigen binding site comprises the variable domains of an antigen binding site of an antibody selected from the group consisting of ReoPro™; Abciximab; Rituxan™; Rituximab; Zenapax™; Daclizumab; Simulect™; Basiliximab; Synagis™; Palivizumab; Remicade™; Infliximab; Herceptin™; Trastuzumab; Mylotarg™; Gemtuzumab; Campath™; Alemtuzumab; Zevalin™; Ibritumomab; Humira™; Adalimumab; Xolair™; Omalizumab; Bexxar™; Tositumomab; Raptiva™; Efalizumab; Erbitux™; Cetuximab; Avastin™; Bevacizumab; Tysabri™; Natalizumab; Actemra™; Tocilizumab; Vectibix™; Panitumumab; Lucentis™; Ranibizumab; Soliris™; Eculizumab; Cimzia™; Certolizumab; Simponi™; Golimumab, Ilaris™; Canakinumab; Stelara™; Ustekinumab; Arzerra™; Ofatumumab; Prolia™; Denosumab; Numax™; Motavizumab; ABThrax™; Raxibacumab; Benlysta™; Belimumab; Yervoy™; Ipilimumab; Adcetris™; Brentuximab; Vedotin™; Perjeta™; Pertuzumab; Kadcyla™; Ado-trastuzumab; Gazyva™ and Obinutuzumab.
32. The method of any preceding paragraph, wherein the target cell is a blood cell.
In an alternative, the target cell is a stem cell or bone marrow cell of a human or animal.
   33. The method of any preceding paragraph, wherein the target cell is a B- or T-cell.
   34. The method of any one of paragraphs 1 to 21 and 31 to 33, wherein the first antigen is an autoimmune disease target and the signaling down-regulates cytotoxic activity or proliferation of the immune cell.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia greata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythaematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

35. The method of any preceding paragraph, wherein the second antigen is an immune cell (eg, human T-cell or NK-cell) extracellular antigen.
In an example, the antigen is comprised by a cell-type that is the same as the type of cell of the invention, eg, the CAL-cell of the invention is a T-cell, NK cell or TIL and the second antigen is a cell surface antigen of T-cells, NK cells or TILS respectively.
36. The method of any preceding paragraph, wherein the second antigen is a protein antigen and the immune cell comprises a first nucleotide sequence that is an endogenous sequence that expresses an amino acid sequence (eg, a CD3 extracellular domain sequence) that is identical to the amino acid sequence of the second antigen comprised by the CAL.
Thus the second antigen is self and recognized by the bridging agent. When the human patient receives the cell, if the patient also expresses the second antigen, this reduces the risk of immune rejection of the CAL-cell by the human.
In an example, the second antigen is provided by a synthetic protein sequence.
37. The method of any preceding paragraph, wherein the second antigen is provided by a human CD3 or human CD16 (eg, CD16A) extracellular domain sequence.
In an example, the second binding site comprises the variable domains of a CD16A binding site of the Tandab™ AFM12 or AFM13.
In an example, the CAL-cell does not express said second antigen from an endogenous nucleotide sequence, eg, wherein the sequence is knocked out or inactivated in the cell genome. In an example, the second antigen is provided by a CD3 or CD16 (eg, CD16A) extracellular domain sequence and the endogenous genome of the CAL-T cell comprises a modification that renders TCR signaling non-functional. For example, the endogenous corresponding CD3 or CD16A extracelluar domain exon sequence has been knocked out or inactivated. In an example, the CD3 extracellular domain is a CD3γ, CD3δ or CDE domain. The skilled addressee will know routine methods for knocking out or modifying sequences precisely in a cell genome, eg by use of homologous recombination and/or CRISPR/Cas (eg, Cas9) nuclease cutting.
38. The method of paragraph 37, wherein the CD3 extracellular domain is a CD3γ, CD3δ or CDE domain.
Optionally, the CD3 extracellular domain is a CD3γ domain. Optionally, the CD3 extracellular domain is a CD3δ domain. Optionally, the CD3 extracellular domain is a CD3E domain.
39. The method of any preceding paragraph, wherein the second target binding site comprises the variable domains of an anti-CD3 binding site of an antibody selected from the group consisting of antibody L2K-07, antibody OKT3™, muromonab-CD3, otelixizumab, teplizumab, visilizumab, catumaxomab, ertumaxomab and foralumab.
40. The method of any preceding paragraph, wherein the extracellular moiety does not comprise non-self epitopes.
This can reduce the risk of rejection when the recipient patient also expresses the domains of the extracellular moiety. In an example, “self” is determined by the phenotype of the patient, eg, human recipient of an immune cell and bridging agent of the invention and/or the phenotype of an ancestor cell from which the immune cell is derived (eg, an ancestor cell obtained from said patient).
41. The method of any preceding paragraph, wherein
A. the second antigen of the CAL is encoded in the cell by a non-endogenous nucleotide (S1) sequence comprising a human single nucleotide polymorphism (SNP1) that encodes an amino acid residue (R1) of the antigen;
B. the genome of the cell comprises a second nucleotide sequence (S2) comprising SNP1 and encoding an amino acid sequence that is identical to the amino acid sequence of the second antigen and comprises R1; or encoding an amino acid sequence that is a naturally-occurring variant of the amino acid sequence of the second antigen and comprises R1; and
C. wherein S2 is an endogenous genomic sequence of the cell and SNP1 is a non-synonymous SNP.
   This provides matching benefits for increased chances of compatibility between the cell and the engineered transmembrane protein (eg, CAL) of the invention, as further discussed herein.
   In an example, the amino acid sequence of the variant is at least 80, 90 or 95% identical to the sequence of the second antigen.
   The term “non-synonymous” SNP is explained above.
   42. The method of any preceding paragraph, wherein
A. the second antigen of the CAL is encoded in the cell by a non-endogenous nucleotide (S1) sequence comprising a human single nucleotide polymorphism (SNP1) that encodes an amino acid residue (R1) of the antigen; and
B. wherein the CAL-cell is comprised by an allogeneic transplant, wherein the method comprises administering the transplant to a human in step (c), wherein the CAL-cell and target cell are combined, wherein the genome of the human comprises said SNP1 before said administration.
   This provides matching benefits as discussed herein.
   A “transplant,” as used herein, refers to cells, tissue, or an organ that is introduced into an individual. The source of the transplanted material can be cultured cells, cells from another individual, or cells from the same individual (e.g., after the cells are cultured in vitro).
   43. The method of paragraph 41 or 42, wherein the second antigen of the CAL is a CD3γ extracellular domain and R1 is selected from the group consisting of I53, N53 and T53 (position numbers correspond to positions of SEQ ID NO: 1).
   44. The method of paragraph 43, wherein the second antigen comprises I53.
   The inventor found that this position shows natural variation in humans and the 153 variant is the most common variation as indicated by Ensembl; thus the inventor realized that this embodiment provides an embodiment that will be matched to most patients receiving the CAL-cell of the invention.
   45. The method of paragraph 41 or 42, wherein the second antigen of the CAL is a CD3δ extracellular domain and R1 is selected from the group consisting of N38 and K38 (position numbers correspond to positions of SEQ ID NO: 3).
   46. The method of paragraph 45, wherein the second antigen comprises N38.
   The inventor found that this position shows natural variation in humans and the N38 variant is the most common variation as indicated by Ensembl; thus inventor realized this embodiment provides an embodiment that will be matched to most patients receiving the CAL-cell of the invention.
   47. The method of paragraph 41 or 42, wherein the second antigen of the CAL is a CD3E extracellular domain and R1 is selected from the group consisting of A108, E108 and V108 (position numbers correspond to positions of SEQ ID NO: 5).
   48. The method of paragraph 47, wherein the second antigen comprises A108.
   The inventor found that this position shows natural variation in humans and the A108 variant is the most common variation as indicated by Ensembl; thus inventor realized this embodiment provides an embodiment that will be matched to most patients receiving the CAL-cell of the invention.
   49. The method according to any one of paragraphs 42 to 48, wherein the CAL-cell is comprised by an allogeneic transplant, wherein the method comprises administering the transplant to a human in step (c), wherein the CAL-cell and target cell are combined, wherein the human comprises said SNP1 before the transplant is administered.
   50. The method according to any one of paragraphs 1 to 48, wherein the CAL-cell is an engineered progeny of an ancestor cell from a human, wherein the method comprises administering the engineered CAL-cell to the human in step (c), wherein the CAL-cell and target cell are combined.
   51. The method of any preceding paragraph, wherein
A. the first signaling domain (SD1) is encoded in the cell by a third nucleotide sequence (S3) comprising a human single nucleotide polymorphism (SNP2) that encodes an amino acid residue (R2) of the signaling domain;
B. the genome of the cell comprises a fourth nucleotide sequence (S4) comprising SNP2 and encoding a signaling domain (SD2), wherein SD2 is identical to SD1 and comprises R2 or (ii) a naturally-occurring variant of SD1 and comprises R2; and
C. wherein S4 is an endogenous genomic sequence of the cell and SNP2 is a non-synonymous SNP.
   52. The method of paragraph 51, wherein each of SD1 and SD2 comprises an immunoreceptor tyrosine-based activation motif (ITAM) comprising R2.
   Given the importance of ITAMs in cell signalling, this aspect of the invention matches the SNP in the ITAM so that it matches the natural situation for functioning with the cell's endogenous signalling machinery.
   53. The method of paragraph 52, wherein the ITAMs of the signaling domains are identical.
   54. The method of any preceding paragraph, wherein the intracellular moiety comprises a further intracellular signaling domain (SD3) that is encoded in the cell by a fifth nucleotide sequence (S5), S5 comprising a human single nucleotide polymorphism (SN P3) that encodes an amino acid residue (R3) of SD3; wherein the genome of the cell comprises a sixth nucleotide sequence (S6) encoding a signaling domain (SD4), wherein SD4 is identical to SD3 and comprises R3; or is a naturally-occurring variant of SD3 and comprises R3; and wherein S6 is an endogenous genomic sequence of the cell and SNP3 is a non-synonymous SNP.
   55. The method of paragraph 54, wherein SD1 and SD3 of the receptor are different.
   56. The method of paragraph 54 or 55, wherein each of SD3 and SD4 comprises an ITAM comprising R3.
   57. The method of paragraph 56, wherein the ITAMs of SD3 and SD4 are identical.
   58. The method of any one of paragraphs 54 to 57, wherein SD3 and SD4 are identical.
   59. The method of any one of paragraphs 54 to 58, wherein SD3 is human.
   60. The method of any one of paragraphs 54 to 59, wherein each of SD3 is a CD3ζ (CD3-zeta) domain, CD3η (CD3-eta) domain, FcεRlγ domain, CD64 domain, CD16 domain, CD27 domain, CD28 domain, ICOS domain, OX40 domain, CD40 domain or 4-1BB domain.
   CD137 is a member of the tumour necrosis factor (TNF) receptor family. Its alternative names are tumour necrosis factor receptor superfamily member 9 (TNFRSF9), 4-1BB and induced by lymphocyte activation (ILA). It is currently of interest to immunologists as a co-stimulatory immune checkpoint molecule.
   61. The method of any preceding paragraph, wherein the first signaling domain is a CD3 intracellular domain selected from a CD3ζ (CD3-zeta) domain (wherein the second antigen is not a CD3 domain), a CD3η (CD3-eta) domain (wherein the second antigen is not a CD3 domain), a FcεRlγ domain, CD64 domain, CD16 domain (wherein the second antigen is not a CD16 domain), CD27 domain, CD28 domain, ICOS domain, OX40 domain, CD40 domain or 4-1BB domain.
   In any aspect herein, the first signaling domain is selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3 and a ligand that specifically binds with CD83.
   62. The method of paragraph 61, wherein the first signaling domain is a CD3 intracellular domain selected from a CD3ζ (CD3-zeta) domain and a CD3η (CD3-eta) domain, and comprises one, two or three amino acid motifs selected from (a) SEQ ID NO: 10 optionally with up to 10, 9, 8, 7, 6 or five (eg, up to 5) amino acid differences, but wherein the tyrosines are conserved; (b) SEQ ID NO: 11 optionally with up to 10, 9, 8, 7, 6 or five (eg, up to 5) amino acid differences, but wherein the tyrosines are conserved; and (c)) SEQ ID NO: 12 optionally with up to 10, 9, 8, 7, 6 or five (eg, up to 5) amino acid differences, but wherein the tyrosines are conserved.
   63. The method of paragraph 62, wherein the first signaling domain comprises motif (a) and wherein the motif differs from SEQ ID NO: 10 by up to five changes at residues selected from the group consisting of A61, P62, A63, Q66, G67, N69, Q70, N73, R79, E82, D84, V85, D87 and K88 (position numbering according to positions in SEQ ID NO: 7).
   This is based on the inventor's analysis of natural human genomic polymorphism in the CD3 zeta sequence and the inventor's realization that these positions are variant naturally, and so permissive for change. Using similar analysis, the inventor also devised other possible variation and matching aspects as in the following paragraphs and the Examples.
   64. The method of paragraph 63, wherein the changes are selected from the group consisting of A61V, A61P, P62S, P62A, A63P, Q66H, G67S, N69T, Q70Y, Q70L, Q70P, Q70W, N73Y, R79L, R79G, E82K, D84G, D84A, D84Y, V851, D87G and K88R (position numbering according to positions in SEQ ID NO: 7).
   The inventor found that these are naturally-occurring variations (and thus are tolerated in humans).
   65. The method of any one of paragraphs 62 to 64, wherein the first signaling domain comprises motif (b) and wherein the motif differs from SEQ ID NO: 11 by up to five changes at residues selected from the group consisting of P100, Q101, R103, K104, N105, P106, E108, L110, A120, A122 and M128 (position numbering according to positions in SEQ ID NO: 7).
   66. The method of paragraph 65, wherein the changes are selected from the group consisting of P100L, Q101L, Q101P, R103K, K104E, N105K, P106R, E108A, L110Q, A120V, A122V and M128T (position numbering according to positions in SEQ ID NO: 7).
   67. The method of any one of paragraphs 62 to 66, wherein the first signaling domain comprises motif (c) and wherein the motif differs from SEQ ID NO: 12 by up to five changes at residues selected from the group consisting of E131, R132, R133, K136, G137, G140, L145, A148, T152, A155, L156 (position numbering according to positions in SEQ ID NO: 7).
   68. The method of paragraph 67, wherein the changes are selected from the group consisting E131K, R132H, R132C, R133Q, R133W, K136N, G137E, G140D, L145F, A148D, T1521, A155T, L156P (position numbering according to positions in SEQ ID NO: 7).
   69. The method of any one of paragraphs 61 to 68, wherein the first signaling domain is a CD3 intracellular domain selected from a CD3ζ (CD3-zeta) domain and a CD3η (CD3-eta) domain, and comprises one, more or all amino acid residues selected from the group consisting of S58, Y64, Y72, Y83, Y111, Y123, Y142 and Y153 (position numbering according to positions in SEQ ID NO: 7).
   The inventor realized that such residues are involved in signaling and recognition in the intracellular pathways, and thus conservation of these residues is useful for good signaling using the transmembrane protein (eg, CAL) of the invention.
   70. The method of any one of paragraphs 61 to 69, wherein the first signaling domain is a CD3 intracellular domain selected from a CD3ζ (CD3-zeta) domain and a CD3η (CD3-eta) domain, and comprises a residue encoded by a SNP selected from the group consisting of r5368651001, r5372651048, r5767112686, r5765877580, r5751145956, r5772867144, r555893506, r5761710510, rs776601547, rs768607376, rs193922741, rs193922740, rs193922739, rs780188126, rs772128174, rs757978223, rs779397562, rs749926653, rs181746205, r5181746205, rs753572867, r5371709798, rs145407267, rs143180729, rs148513413, rs367690333, rs144963570, rs367690333, rs144963570, r5770320255, r5139926301, rs56297636, r5760895755, r5112890541, r5370910340, r5145505909, r5754935006, r5751583971, r5766541481, r5763074967, r5745871212, r5372665461, r5764185491, r5756340039, r5773572491, r5201594815, r5781510519, r5147527561, r5751981677, rs763532939, rs753278244, r5771873949, r5186004179, r5186004179, rs762773775, r5748158220, r5776703680, r5561262982, r5758846009, r5746262183, r5376046446, r5201937405 and r5752198795; and wherein the genome of the immune cell comprises an endogenous nucleotide sequence comprising said selected SNP.
   The inventor realized that these SNPs encode residues at positions in naturally-occurring CD3-zeta and CD3-eta that are permissive for change. It is useful, for example where the cell's endogenous genome also comprises the SNP, in order to provide matching benefits as discussed herein.
   71. The method of any preceding paragraph, wherein the first or third signaling domain is a CD28 intracellular domain comprising at least 13 amino acid residues selected from the group consisting of R180, 5181, K182, R183, 5184, R185, L186, D190, Y191, N193, P196, P199, T202, K204, Q207, F215, A217 and Y218 (position numbers correspond to positions of SEQ ID NO: 13).
   The inventor realized that these are naturally-occurring variations in the CD28 intracelluar domain, and thus form a basis for matching as per the matching aspect of the invention. It is useful, for example where the cell's endogenous genome also encodes the selected residue, in order to provide matching benefits as discussed herein.
   72. The method of paragraph 71, wherein the CD28 domain comprises all residues of said group.
   73. The method of paragraph 71 or 72, wherein the CD28 domain comprises amino acid residues Y191 and Y209 (position numbers correspond to positions of SEQ ID NO: 13).
   As described further in the Examples, the inventor realized that these positions appear in conserved CD28 intracellular motifs (YMNM and PYAP) that are important for intracellular signaling.
   74. The method of any one of paragraphs 71 to 73 wherein the CD28 domain comprises a YMNM motif (corresponding to Y191-M192-N193-M194 of SEQ ID NO: 13) and/or a PYAP motif (corresponding to P208-Y209-A210-P211 of SEQ ID NO: 13).
   75. The method of any preceding paragraph, wherein the CAL-immune cell is a CAL-T-cell (eg, CD8⁺ T-cell or CD4⁺ T-cell, eg, an activated T-cell), NK cell, tumour-infiltrating lymphocyte (TIL, eg, a pre-REP TIL), memory T-cell, T_{SCM}, T_{CM} or T_{EM}.

Within the overall memory T cell population, several distinct subpopulations have been described and can be recognised by the differential expression of chemokine receptor CCR7 and L-selectin (CD62L). Stem memory T_{SCM} cells, like naive cells, are CD45RO−, CCR7+, CD45RA+, CD62L+(L-selectin), CD27+, CD28+ and IL-7Ra+, but they also express large amounts of CD95, IL-2Rβ, CXCR3, and LFA-1, and show numerous functional attributes distinctive of memory cells. Central memory T_{CM} cells express L-selectin and the CCR7, they secrete IL-2, but not IFNγ or IL-4. Effector memory T_{EM} cells, however, do not express L-selectin or CCR7 but produce effector cytokines like IFNγ and IL-4. Memory T-cells, such as T_{SCM} may be particularly useful for establishing a sustained population of engineered immune cells in the human.

Any stem cell herein can, in an example, be a T_{SCM}, T_{CM} or T_{EM} cell, eg, a human T_{SCM}, T_{CM} or T_{EM} cell.

76. The method of any preceding paragraph, wherein the CAL-immune cell is a progeny of a cell of a human suffering from an autoimmune disease, an inflammatory disease, a viral infection or a cancer, eg, wherein the human is suffering from lymphoblastic leukaemia, ALL (eg, T-ALL), CLL (eg, B-cell chronic lymphocytic leukaemia) or non-Hodgkin's lymphoma.
77. The method of any preceding paragraph, wherein the CAL-immune cell has been engineered for enhanced signaling, wherein the signaling is selected from CD28, 4-1BB, OX40, ICOS and CD40 signaling.
78. The method of any preceding paragraph, wherein step (c) comprises mixing the cells together before combining the mixed cells with the bridging agent.
79. The method of any one of paragraphs 1 to 77, comprising mixing the target cell and bridging agent together before combing the target cell and agent with the immune cell.
80. The method of any preceding paragraph, wherein the method is carried out with a plurality of said target cells, a plurality of said CAL-immune cells and multiple copies of said bridging agent, wherein in step (c) the target cells, CAL-cells and agent copies are combined and activity of said immune cells is thereby regulated.
81. The method of paragraph 80, wherein the amount of agent is reduced after step (c).
82. The method of paragraph 80, wherein more of said agent is combined after step (c).
83. The method of any one of paragraphs 80 to 82 carried out in a human or animal.
84. The method of any one of paragraphs 80 to 82 carried out using target and CAL-cells in vitro.
85. The method of any one of paragraphs 80 to 84 for treating or reducing the risk of a disease or condition in a human comprising target cells, wherein step (c) comprises simultaneously or sequentially administering the bridging agent copies and the CAL-cells (or ancestor cells thereof) to the human, wherein the bridging agent binds to the first and second antigens to target the CAL-cells to the target cells, thereby triggering intracellular signaling in the CAL-cells to regulate CAL-cell activity, whereby the disease or condition is treated or the risk of the disease or condition is reduced.
86. The method of paragraph 85, wherein the disease or condition is a cancer, inflammatory disease, autoimmune disease or a viral infection in the human.
87. The method of paragraph 85 or 86, wherein target cells (eg, tumour cells) are killed.
88. The method of paragraph 86 or 87, wherein each target cell is a tumour cell and the method treats or reduces the risk of cancer, or treats or reduces the risk of cancer progression in the human.
89. The method of any one of paragraphs 85 to 88, wherein the human has cancer.
In an example, the cancer is a haematological cancer. In an example, the human has a cancer of B-cell origin. In an example, the human has a cancer of T-cell origin.
For example the cancer is lung cancer, melanoma, breast cancer, prostate cancer, colon cancer, renal cell carcinoma, ovarian cancer, neuroblastoma, rhabdomyosarcoma, leukaemia and lymphoma. Preferred cancer targets for use with the present invention are cancers of B cell origin, particularly including acute lymphoblastic leukaemia, B-cell chronic lymphocytic leukaemia or B-cell non-Hodgkin's lymphoma.
   90. The method of paragraph 89, wherein the cancer is a cancer of T-cell or B-cell origin, eg, lymphoblastic leukaemia, ALL (eg, T-ALL), CLL (eg, B-cell chronic lymphocytic leukaemia) or non-Hodgkin's lymphoma.
   91. The method of any one of paragraphs 85 to 90, wherein the CAL-cell is a progeny of an immune cell of said human, eg, wherein the human is suffering from lymphoblastic leukaemia, Diffuse Large B-cell Lymphoma (DLBCL), ALL (eg, T-ALL or B-ALL), CLL (eg, B-cell chronic lymphocytic leukaemia) or non-Hodgkin's lymphoma.
   92. The method of any one of paragraphs 85 to 91, wherein each said cell is an autologous cell (eg, T-cell) of said human or is a progeny of such an autologous cell.
As used herein, the term “autologous” is meant to refer to any material derived from the same individual to which it is later to be re-introduced into the individual.
“Allogeneic” refers to a graft derived from a different animal of the same species.
   93. The method of paragraph 92, wherein each CAL-cell is derived from a blood or tumour sample of the human and activated and expanded in vitro before step (c).
“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.
   94. The method of paragraph 80, wherein the human has an autoimmune disease, wherein the CAL-cells are anergic, or have reduced proliferation and/or cytotoxic activity when bound to target cells by the bridging agent copies, whereby the CAL-cells compete with endogenous immune cells of said human that up-regulate said autoimmune disease.
   95. The method of any one of paragraphs 85 to 94, wherein administration is by CAL-cell infusion into the blood of the patient.
   96. The method of any one of paragraphs 85 to 95, comprising expanding the CAL-immune cells to produce an expanded immune cell population that is combined with target cells in step (c).
   97. The method of any preceding paragraph, comprising activating the engineered immune cell(s) to produce an activated immune cell population that is combined with target cell(s) in step (c).
   98. A chimaeric antigen ligand (CAL)-immune cell for targeted binding to an antigen-specific agent,
A. wherein the agent is a multi-specific antigen binding fragment comprising
   i. a first antigen binding site that specifically binds a first target antigen; and
   ii. a second antigen binding site that specifically binds a second target antigen;
B. wherein the CAL-immune cell comprises a transmembrane ligand, the ligand comprising an engineered combination of
   iii. an extracellular moiety comprising the second antigen, wherein the second antigen is linked to a transmembrane domain; and
   iv. an intracellular moiety comprising a first signalling domain for intracellular signalling when the agent binds to the second antigen;
C. wherein when the CAL-immune cell and bridging agent are combined with a target cell, the target cell comprising said first target antigen, wherein the first antigen is an extracellular antigen,
   v. the bridging agent binds to the first and second antigens to target the immune cell to the target cell,
   vi. thereby triggering intracellular signalling in the immune cell to regulate immune cell activity.
      The features of methods 1 to 97 can apply mutatis mutandis to the features of paragraphs 98 onwards.
      In an alternative, paragraph 98 provides the following alternative clauses VIII to XV. Hence, features of paragraphs 99 onwards below are combinable with any of clauses VIII to XV. Reference to “paragraph 98” below can in the alternative be read as any one of clauses VIII to XV; “first antigen binding site” in any one of paragraphs 99 onwards can be read as “first binding moiety” as recited in any of any of clauses VIII to XV; “second antigen binding site” in any one of paragraphs 99 onwards can be read as “second binding moiety” as recited in any of any of clauses VIII to XV; “first antigen” in any one of paragraphs 99 onwards can be read as “fourth binding moiety” as recited in any of any of clauses VIII to XV; “second antigen” in any one of paragraphs 99 onwards can be read as “third binding moiety” as recited in any of any of clauses VIII to XV; and CAL in any one of paragraphs 99 onwards can be read as the “transmembrane protein”, “CAR” or “CAL” of any of clauses VIII to XV.
      VIII. An immune cell for targeted binding to an antigen-specific agent,
A. wherein the agent is a multi-specific binding fragment comprising
   i. a first binding moiety; and
   ii. a second binding moiety;
B. wherein the immune cell expresses a transmembrane protein comprising an engineered combination of
   iii. an extracellular part comprising a third binding moiety that is linked to a transmembrane domain; wherein the second and third moieties form a specific binding pair (SBP1) wherein one moiety specifically binds to the other moiety; and
   iv. an intracellular part comprising a first signaling domain for intracellular signaling when the second and third moieties bind together;
C. wherein when the immune cell and bridging agent are combined with a target cell, the target cell comprising a fourth binding moiety, wherein the fourth moiety is extracellular,
   v. the first and fourth moieties form a specific binding pair (SBP2) wherein one moiety specifically binds to the other moiety to target the immune cell to the target cell;
   vi. the second and third moieties bind together thereby triggering intracellular signaling in the immune cell to regulate immune cell activity; and
D. wherein the molecular weight of the bridging agent is no more than 125 kDa.
   IX. The immune cell of clause VIII in combination with the bridging agent.
   X. The immune cell of clause VIII of IX, wherein the first and second binding moieties are ligand binding sites.
   XI. The immune cell of X, wherein the first and second binding moieties are first and second antigen binding sites respectively (eg, VH/VL antigen binding sites).
   XII. The immune cell of any one of clauses VIII to XI, wherein each of SBP1 and SB2 is selected from the group consisting of:—
   (a) an antigen and an antigen binding site (eg, an antigen and a VH/VL antigen binding site; or a superantigen and an antibody variable domain); and
   (b) a receptor and a ligand.
   XIII. The immune cell of any one of clauses VIII to XII, wherein the transmembrane protein is a chimaeric antigen ligand (CAL), wherein the third moiety is an antigen and the second moiety is an antigen binding site (eg, an scFv); and optionally the first moiety is an antigen binding site (eg, an scFv) and the fourth moiety is an antigen.
   XIV. The immune cell of any one of clauses VIII to XII, wherein the transmembrane protein is a chimaeric antigen receptor (CAR), wherein the third moiety is an antigen binding site and the second moiety is an antigen; and optionally the first moiety is an antigen binding site (eg, an scFv) and the fourth moiety is an antigen.
   XV. The immune cell of any one of clauses VIII to XIV, wherein the molecular weight of the bridging agent is no more than 115 kDa, eg, from 55 to 115 kDa, or from 60 to 100 kDa.
   Generally-applicable features:—
   99. The CAL-cell of paragraph 98, wherein the CAL comprises a hinge region and/or a linker between the second antigen and the transmembrane domain.
   100. The CAL-cell of paragraph 99, wherein the hinge region is CD8α hinge region.
   In an example, the transmembrane domain is a CD8α transmembrane domain. In an example, the second antigen and/or first signaling domain are human; optionally the transmembrane domain and/or hinge is human.
   101. The CAL-cell of any one of paragraphs 98 to 100, wherein the signalling upregulates CAL-cell cytotoxicity.
   102. The CAL-cell of any one of paragraphs 98 to 101, wherein the signalling upregulates CAL-cell ADCC activity.
   103. The CAL-cell of any one of paragraphs 98 to 102, wherein the first antigen is a tumour associated antigen (TAA).
   104. The CAL-cell of any one of paragraphs 98 to 103, wherein the first antigen is human CD19 (and optionally the target cell is a leukaemic or lymphoma cell), EpCAM (and optionally the target cell is a lung cancer cell, gastrointestinal cancer cell, an adenocarcinoma, cancer stem cell), CD20 (and optionally the target cell is a leukaemic cell), MCSP (and optionally the target cell is a melanoma cell), CEA, EGFR, EGFRvIII, HER2, sialyl Tn, CD133, CD33 (and optionally the target cell is a leukaemic cell, eg, AML cell), PMSA, CD30, CD47, CD52, gpA33, TAG-72, mucin, CIX, GD2, GD3, GM2, CD123, VEGFR, integrin, cMET, Her1, Her2, Her3, IGF1R, EPHA3, CD66e, EphA2, TRAILR1, TRAILR2, RANKL, FAP, Angiopoietin or tenascin.
   105. The CAL-cell of any one of paragraphs 98 to 104, wherein the first antigen binding site comprises the variable domains of an antibody selected from the group consisting of the CD19 binding site of blinatumomab or antibody HD37; EpCAM binding site of Catumaxomab; CD19 binding site of AFM11; CD20 binding site of Lymphomun; Her2 binding site of Ertumaxomab; CEA binding site of AMG211 (MEDI-565, MT111); PSMA binding site of Pasotuxizumab; EpCAM binding site of solitomab; VEGF or angiopoietin 2 binding site of RG7221 or RG7716; Her1 or Her3 binding site of RG7597; Her2 or Her3 binding site of MM111; IGF1R or Her3 binding site of MM141; CD123 binding site of MGD006; gpa33 binding site of MGD007; CEA binding site of TF2; CD30 binding site of AFM13; CD19 binding site of AFM11; and Her1 or cMet binding site of LY3164530.
   106. The CAL-cell of any one of paragraphs 98 to 105, wherein the bridging agent is blinatumomab or a CD3/CD19-binding derivative thereof (optionally wherein the target cell is an acute lymphoblastic leukaemia (ALL) B-cell); AMG211 or a CD3/CEA-binding derivative thereof (MEDI-565, MT111; optionally wherein the target cell is a Gastrointestinal cancer cell); Pasotuxizumab or a CD3/PMSA-binding derivative thereof (optionally wherein the target cell is a prostate cancer cell); solitomab or a CD3/EpCAM-binding derivative thereof (optionally wherein the target cell is a cancer cell); or AFM11 or a CD3/CD19-binding derivative thereof (and optionally wherein the target cell is an ALL cell or Non-Hodgkins Lymphoma cell).
   107. The CAL-cell of any one paragraphs 98 to 106, the first antigen binding site comprises the variable domains of an antigen binding site of an antibody selected from the group consisting of ReoPro™; Abciximab; Rituxan™; Rituximab; Zenapax™; Daclizumab; Simulect™; Basiliximab; Synagis™; Palivizumab; Remicade™; Infliximab; Herceptin™; Trastuzumab; Mylotarg™; Gemtuzumab; Campath™; Alemtuzumab; Zevalin™; Ibritumomab; Humira™; Adalimumab; Xolair™; Omalizumab; Bexxar™; Tositumomab; Raptiva™; Efalizumab; Erbitux™; Cetuximab; Avastin™; Bevacizumab; Tysabri™; Natalizumab; Actemra™; Tocilizumab; Vectibix™; Panitumumab; Lucentis™; Ranibizumab; Soliris™; Eculizumab; Cimzia™; Certolizumab; Simponi™; Golimumab, Ilaris™; Canakinumab; Stelara™; Ustekinumab; Arzerra™; Ofatumumab; Prolia™; Denosumab; Numax™; Motavizumab; ABThrax™; Raxibacumab; Benlysta™; Belimumab; Yervoy™; Ipilimumab; Adcetris™; Brentuximab; Vedotin™; Perjeta™; Pertuzumab; Kadcyla™; Ado-trastuzumab; Gazyva™ and Obinutuzumab.
   108. The CAL-cell of any one of paragraphs 98 to 102, wherein the first antigen is an autoimmune disease target and the signalling down-regulates cytotoxic activity or proliferation of the immune cell.
   109. The CAL-cell of any one of paragraphs 98 to 108, wherein the second antigen is an immune cell (eg, human T-cell or NK-cell) extracellular antigen.
   110. The CAL-cell of any one of paragraphs 98 to 109, wherein the second antigen is a protein antigen and the immune cell comprises a first nucleotide sequence that is an endogenous sequence that expresses an amino acid sequence (eg, a CD3 extracellular domain sequence) that is identical to the amino acid sequence of the second antigen comprised by the CAL.
   111. The CAL-cell of any one of paragraphs 98 to 110, wherein the second antigen is provided by a human CD3 or human CD16 extracellular domain sequence.
   112. The CAL-cell of paragraph 111, wherein the CD3 extracellular domain is a CD3γ, CD3δ or CDE domain.
   113. The CAL-cell of any one of paragraphs 98 to 112, wherein the second target binding site comprises the variable domains of an anti-CD3 binding site of an antibody selected from the group consisting of antibody L2K-07, antibody OKT3™, muromonab-CD3, otelixizumab, teplizumab, visilizumab, catumaxomab, ertumaxomab and foralumab.
   114. The CAL-cell of any one of paragraphs 98 to 113, wherein the extracellular moiety does not comprise non-self epitopes.
   115. The CAL-cell of any one of paragraphs 98 to 114, wherein
a. the second antigen of the CAL is encoded in the cell by a non-endogenous nucleotide (S1) sequence comprising a human single nucleotide polymorphism (SNP1) that encodes an amino acid residue (R1) of the antigen;
b. the genome of the cell comprises a second nucleotide sequence (S2) comprising SNP1 and encoding an amino acid sequence that is identical to the amino acid sequence of the second antigen and comprises R1; or encoding an amino acid sequence that is a naturally-occurring variant of the amino acid sequence of the second antigen and comprises R1; and
c. wherein S2 is an endogenous genomic sequence of the cell and SNP1 is a non-synonymous SNP.
   116. The CAL-cell of any one of paragraphs 98 to 115, wherein the CAL-cell is for use in a method of treating or reducing the risk of a disease or condition (eg, a cancer) in a human, wherein the method comprises administering the CAL-cell and said bridging agent to the human; wherein the CAL-cell and a target cell of the human are combined and bridged by the bridging agent, thereby up-regulating signalling in the CAL-cell to enhance target cell cytoxicity (eg, ADCC-mediated killing activity) of the CAL-cell, thereby treating or reducing the risk of said disease or condition in the human.
   In methods herein, an effective amount of CAL-cells and bridging agent are administered. An “effective amount” as used herein, means an amount which provides a therapeutic or prophylactic benefit.
   In an embodiment of the method of the invention, the method treats or reduces the risk of cancer in a patient (eg, a human), wherein the patient has undergone lymphodepletion before administration of the immune cells of the invention to the patient.
   117. The CAL-cell of any one of paragraphs 98 to 115, wherein the CAL-cell is for use in a method of treating or reducing the risk of a disease or condition (eg, an autoimmune disease, GvHD or allogenic transplant rejection) in a human, wherein the method comprises administering the CAL-cell and said bridging agent to the human; wherein the CAL-cell and a target cell of the human are combined and bridged by the bridging agent, thereby up-regulating signalling in the CAL-cell to reduce cytoxicity (eg, ADCC-mediated killing activity) of the CAL-cell, thereby treating or reducing the risk of said disease or condition in the human.
   118. The CAL-cell of paragraph 117, wherein said bridging triggers CAL-cell anergy in the human.
   119. A transplant (eg, an allogenic transplant) comprising a plurality of CAL-cells according to paragraph 117 or 118, wherein the transplant is for competing with endogenous disease- or condition-mediating immune cells of the human (eg, mediated by T-cell activity) to treat or reduce said disease or condition.
For example, the CAL-cells effectively “dilute” the prevalence of disease-mediating endogenous immune cells at sites of tissue damage or other disease activity. By reducing the local concentration of said disease-mediating endogenous immune cells in this way, the undesirable activity (eg, autoimmune, GvHD or rejection activity) of endogenous immune cells, such as T-cells, may be dampened down in the human. In this case, in one embodiment it is advantageous that the CAL-cells (eg, CAL-T cells) become anergic, whereby such anergic cells “dilute” activity of endogenous T-cells and thus dampen down the disease or condition or reduce the risk of the disease or condition in the human.
In another example, the immune cells of the invention become activated to kill target cells (eg, T-cells) of the patient to treat or prevent the disease or condition.
To “treat” a disease as the term is used herein, means to reduce the frequency or severity of at least one sign or symptom of a disease or condition experienced by a subject.
   120. The CAL-cell or transplant of any one of paragraphs 116 to 119 when dependent from paragraph 115, wherein the genome of the human comprises said SNP1 before said administration.
   In this way, the cell or transplant is matched in the extracellular moiety to increase compatibility with the recipient human.
   121. The CAL-cell or transplant of any one of paragraphs 98 to 120, wherein the target cell is a human cell.
   122. The CAL-cell or transplant of any one of paragraphs 98 to 121, wherein the target cell is a tumour cell and the signalling up-regulates cytotoxic activity (eg, ADCC) or proliferation of the immune cell.
   123. The CAL-cell or transplant of any one of paragraphs 98 to 122, wherein the target cell is a leukaemic cell, lymphoma cell, adenocarcinoma cell or cancer stem cell.
   124. The CAL-cell or transplant of any one of paragraphs 98 to 123, wherein the target cell is a blood cell.
   125. The CAL-cell or transplant of any one of paragraphs 98 to 123, wherein the target cell is a B- or T-cell.
   126. The CAL-cell or transplant of paragraph 115 or any one of paragraphs 116 to 125 when dependent from paragraph 115, wherein the second antigen of the CAL is a CD3γ extracellular domain and R1 is selected from the group consisting of 111, M11, A22, T22, 153, N53, T53, V131, F131, R166, G166, Y171 and H171 (position numbers correspond to positions of SEQ ID NO: 1).
   As with aspects above, the inventor realized that such positions are permissive for CD3γ variation naturally occurring in humans, and thus the inventor identified these as candidate positions for matching according to the invention.
   127. The CAL-cell or transplant of paragraph 126, wherein the second antigen comprises 111, A22, 153, N53, V131, R166 and Y171.
   The inventor realised that these variations are most common in humans (as indicated by Ensembl) and thus this feature is likely to be compatible with most humans and most human cells.
   128. The CAL-cell or transplant of paragraph 115 or any one of paragraphs 116 to 125 when dependent from paragraph 115, wherein the second antigen of the CAL is a CD3δ extracellular domain and R1 is selected from the group consisting of Q18, K18, N38 and K38 (position numbers correspond to positions of SEQ ID NO: 3).
   As with aspects above, the inventor realized that such positions are permissive for CD3δ variation naturally occurring in humans, and thus the inventor identified these as candidate positions for matching according to the invention.
   129. The CAL-cell or transplant of paragraph 128, wherein the second antigen comprises Q18 and N38.
   The inventor realised that these variations are most common in humans (as indicated by Ensembl) and thus this feature is likely to be compatible with most humans and most human cells.
   130. The CAL-cell or transplant of paragraph 115 or any one of paragraphs 116 to 125 when dependent from paragraph 115, wherein the second antigen of the CAL is a CD3E extracellular domain and R1 is selected from the group consisting of D71, N71, H71, Y71, A108, E108, V108, A157 and V157 (position numbers correspond to positions of SEQ ID NO: 5).
   As with aspects above, the inventor realized that such positions are permissive for CD3E variation naturally occurring in humans, and thus the inventor identified these as candidate positions for matching according to the invention.
   131. The CAL-cell or transplant of paragraph 130, wherein the second antigen comprises D71, A108 and A157.
   The inventor realised that these variations are most common in humans (as indicated by Ensembl) and thus this feature is likely to be compatible with most humans and most human cells.
   132. The CAL-cell or transplant of any one of paragraphs 116 to 131, wherein each CAL-cell is an engineered progeny of an ancestor cell from the human, wherein the method comprises administering the engineered CAL-cell or transplant to the human, wherein the CAL-cell(s) and target cell are combined.
   133. The CAL-cell or transplant of any one of paragraphs 98 to 132, wherein
a. the first signalling domain (SD1) is encoded in each CAL-cell by a third nucleotide sequence (S3) comprising a human single nucleotide polymorphism (SNP2) that encodes an amino acid residue (R2) of the signalling domain;
b. the genome of the cell comprises a fourth nucleotide sequence (S4) comprising SNP2 and encoding a signalling domain (SD2), wherein SD2 is identical to SD1 and comprises R2 or (ii) a naturally-occurring variant of SD1 and comprises R2; and
c. wherein S4 is an endogenous genomic sequence of the cell and SNP2 is a non-synonymous SNP.
   134. The CAL-cell or transplant of paragraph 133, wherein each of SD1 and SD2 comprises an immunoreceptor tyrosine-based activation motif (ITAM) comprising R2.
   135. The CAL-cell or transplant of paragraph 134, wherein the ITAMs of the signalling domains are identical.
   136. The CAL-cell or transplant of any one of paragraphs 98 to 135, wherein the intracellular moiety comprises a further intracellular signalling domain (SD3) that is encoded in the cell by a fifth nucleotide sequence (S5), S5 comprising a human single nucleotide polymorphism (SNP3) that encodes an amino acid residue (R3) of SD3; wherein the genome of the cell comprises a sixth nucleotide sequence (S6) encoding a signalling domain (SD4), wherein SD4 is identical to SD3 and comprises R3; or is a naturally-occurring variant of SD3 and comprises R3; and wherein S6 is an endogenous genomic sequence of the cell and SNP3 is a non-synonymous SNP.
   137. The CAL-cell or transplant of paragraph 136, wherein SD1 and SD3 of the receptor are different.
   138. The CAL-cell or transplant of paragraph 136 or 137, wherein each of SD3 and SD4 comprises an ITAM comprising R3.
   139. The CAL-cell or transplant of paragraph 138, wherein the ITAMs of SD3 and SD4 are identical.
   140. The CAL-cell or transplant of any one of paragraphs 136 to 139, wherein SD3 and SD4 are identical.
   141. The CAL-cell or transplant of any one of paragraphs 136 to 140, wherein SD3 is human.
   142. The CAL-cell or transplant of any one of paragraphs 136 to 141, wherein each of SD3 is a CD3ζ (CD3-zeta) domain, CD3η (CD3-eta) domain, FcεRlγ domain, CD64 domain, CD16 (eg, CD16A) domain, CD27 domain, CD28 domain, ICOS domain, OX40 domain, CD40 domain or 4-1BB domain.
   143. The CAL-cell or transplant of any one of paragraphs 98 to 142, wherein the first signalling domain is a CD3 intracellular domain selected from a CD3ζ (CD3-zeta) domain (wherein the second antigen is not a CD3 domain), a CD3η (CD3-eta) domain (wherein the second antigen is not a CD3 domain), a FcεRlγ domain, CD64 domain, CD16 domain (wherein the second antigen is not a CD16 domain), CD27 domain, CD28 domain, ICOS domain, OX40 domain, CD40 domain or 4-1BB domain.
   144. The CAL-cell or transplant of paragraph 143, wherein the first signalling domain is a CD3 intracellular domain selected from a CD3ζ (CD3-zeta) domain and a CD3η (CD3-eta) domain, and comprises one, two or three amino acid motifs selected from (a) SEQ ID NO: 10 optionally with up to 10, 9, 8, 7, 6 or five (eg, up to 5) amino acid differences, but wherein the tyrosines are conserved; (b) SEQ ID NO: 11 optionally with up to 10, 9, 8, 7, 6 or five (eg, up to 5) amino acid differences, but wherein the tyrosines are conserved; and (c)) SEQ ID NO: 12 optionally with up to 10, 9, 8, 7, 6 or five (eg, up to 5) amino acid differences, but wherein the tyrosines are conserved.
   145. The CAL-cell or transplant of paragraph 144, wherein the first signalling domain comprises motif (a) and wherein the motif differs from SEQ ID NO: 10 by up to five changes at residues selected from the group consisting of A61, P62, A63, Q66, G67, N69, Q70, N73, R79, E82, D84, V85, D87 and K88.
   146. The CAL-cell or transplant of paragraph 145, wherein the changes are selected from the group consisting of A61V, A61P, P62S, P62A, A63P, Q66H, G67S, N69T, Q70Y, Q70L, Q70P, Q70W, N73Y, R79L, R79G, E82K, D84G, D84A, D84Y, V851, D87G and K88R.
   147. The CAL-cell or transplant of any one of paragraphs 144 to 146, wherein the first signalling domain comprises motif (b) and wherein the motif differs from the positions SEQ ID NO: 12 by up to five changes at residues selected from the group consisting of P100, Q101, R103, K104, N105, P106, E108, L110, A120, A122 and M128.
   148. The CAL-cell or transplant of paragraph 147, wherein the changes are selected from the group consisting of P100L, Q101L, Q101P, R103K, K104E, N105K, P106R, E108A, L110Q, A120V, A122V and M128T.
   149. The CAL-cell or transplant of any one of paragraphs 144 to 148, wherein the first signalling domain comprises motif (c) and wherein the motif differs from SEQ ID NO: 13 by up to five changes at residues selected from the group consisting of E131, R132, R133, K136, G137, G140, L145, A148, T152, A155, L156.
   150. The CAL-cell or transplant of paragraph 149, wherein the changes are selected from the group consisting E131K, R132H, R132C, R133Q, R133W, K136N, G137E, G140D, L145F, A148D, T1521, A155T, L156P.
   151. The CAL-cell or transplant of any one of paragraphs 98 to 150, wherein the first signalling domain is a CD3 intracellular domain selected from a CD3ζ (CD3-zeta) domain and a CD3η (CD3-eta) domain, and comprises one, more or all amino acid residues selected from the group consisting of S58, Y64, Y72, Y83, Y111, Y123, Y142 and Y153 (position numbers correspond to positions of SEQ ID NO: 7).
   152. The CAL-cell or transplant of any one of paragraphs 98 to 151, wherein the first signalling domain is a CD3 intracellular domain selected from a CD3ζ (CD3-zeta) domain and a CD3η (CD3-eta) domain, and comprises a residue encoded by a SNP selected from the group consisting of r5368651001, r5372651048, r5767112686, r5765877580, r5751145956, r5772867144, r555893506, rs761710510, rs776601547, rs768607376, rs193922741, rs193922740, rs193922739, rs780188126, r5772128174, rs757978223, rs779397562, rs749926653, r5181746205, r5181746205, rs753572867, r5371709798, r5145407267, r5143180729, r5148513413, r5367690333, r5144963570, r5367690333, r5144963570, r5770320255, r5139926301, rs56297636, r5760895755, r5112890541, r5370910340, r5145505909, r5754935006, r5751583971, r5766541481, r5763074967, r5745871212, r5372665461, rs764185491, rs756340039, rs773572491, rs201594815, rs781510519, rs147527561, rs751981677, rs763532939, rs753278244, r5771873949, r5186004179, r5186004179, rs762773775, r5748158220, r5776703680, r5561262982, r5758846009, r5746262183, r5376046446, r5201937405 and rs752198795; and wherein the genome of the immune cell comprises an endogenous nucleotide sequence comprising said selected SNP.
   153. The CAL-cell or transplant of any one of paragraphs 98 to 152, wherein the first or third signalling domain is a CD28 intracellular domain comprising at least 13 amino acid residues selected from the group consisting of R180, 5181, K182, R183, 5184, R185, L186, D190, Y191, N193, P196, P199, T202, K204, Q207, F215, A217 and Y218 (position numbers correspond to positions of SEQ ID NO: 13).
   154. The CAL-cell or transplant of paragraph 153, wherein the CD28 domain comprises all residues of said group.
   155. The CAL-cell or transplant of paragraph 153 or 154, wherein the CD28 domain comprises amino acid residues Y191 and Y209 (position numbers correspond to positions of SEQ ID NO: 13).
   156. The CAL-cell or transplant of any one of paragraphs 153 to 155 wherein the CD28 domain comprises a YMNM motif (corresponding to Y191-M192-N193-M194 of SEQ ID NO: full length CD28 seq) and/or a PYAP motif (corresponding to P208-Y209-A210-P211 of SEQ ID NO: 13).
   157. The CAL-cell or transplant of any one of paragraphs 98 to 156, wherein each CAL-immune cell is a CAL-T-cell (eg, CD8+ T-cell or CD4+ T-cell, eg, an activated T-cell), NK cell, tumour-infiltrating lymphocyte (TIL, eg, a pre-REP TIL), memory T-cell, TSCM, TCM or TEM.
   158. The CAL-cell or transplant of any one of paragraphs 98 to 157, wherein each CAL-immune cell is a progeny of a cell of a human suffering from an autoimmune disease, an inflammatory disease, a viral infection or a cancer, eg, wherein the human is suffering from lymphoblastic leukaemia, ALL (eg, T-ALL), CLL (eg, B-cell chronic lymphocytic leukaemia) or non-Hodgkin's lymphoma.
In one embodiment, the human is resistant to at least one chemotherapeutic agent.
In one embodiment, the chronic lymphocytic leukaemia is refractory CD 19+ leukaemia and lymphoma.
The invention also includes a method of generating a persisting population of genetically engineered T cells in a human diagnosed with cancer. In one embodiment, the method comprises administering to a human an T-cell of the invention (eg, a CAL T-cell), wherein the persisting population of genetically engineered T cells persists in the human for at least one month after administration. In one embodiment, the persisting population of genetically engineered T cells comprises a memory T-cell. In one embodiment, the persisting population of genetically engineered T-cells persists in the human for at least three months after administration. In another embodiment, the persisting population of genetically engineered T-cells persists in the human for at least four months, five months, six months, seven months, eight months, nine months, ten months, eleven months, twelve months, two years, or three years after administration.
In one embodiment, the chronic lymphocytic leukaemia is treated. The invention also provides a method of expanding a population of the engineered T-cells or NK cells in a human diagnosed with cancer.
Optionally, autologous lymphocyte infusion is used in the treatment. Autologous PBMCs are collected from a patient in need of treatment and T-cells are engineered to express the transmembrane protein of the invention, activated and expanded using the methods known in the art and then infused back into the patient simultaneously or sequentially with administration of the bridging agent.
   159. The CAL-cell or transplant of any one of paragraphs 98 to 158, wherein each CAL-immune cell has been engineered for enhanced signalling, wherein the signalling is selected from CD28, 4-1BB, OX40, ICOS and CD40 signalling.
   160. The CAL-cell or transplant of any one of paragraphs 98 to 159, wherein each CAL-cell is derived from a blood or tumour sample of a human (eg, a cancer patient) and the cell is an activated cell.
   161. The CAL-cell or transplant of any one of paragraphs 98 to 160 in combination with the bridging agent.
   162. The CAL-cell or transplant of paragraph 161, wherein the bridging agent has a human serum half-life that is less than the human serum half-life of IgG.
   163. The CAL-cell or transplant of paragraph 161 or 162, wherein the bridging agent has a human serum half-life of no more than 5 days.
   164. The CAL-cell or transplant of paragraph 163, wherein the half-life is less than 1 day.
   165. The CAL-cell or transplant of any one of paragraphs 161 to 164, wherein the first binding site is an antibody VH/VL binding site.
   166. The CAL-cell or transplant of any one of paragraphs 161 to 165, wherein the second binding site is an antibody VH/VL binding site.
   167. The CAL-cell or transplant of any one of paragraphs 161 to 166, wherein the binding affinity (KD) of the first binding site for the first antigen is at least 5-, 10- or 20-fold lower than the affinity of the second binding site for the second antigen.
   168. The CAL-cell or transplant of any one of paragraphs 161 to 167, wherein the first binding site has a binding affinity (KD) for the first antigen of 10 nM or less as determined by surface plasmon resonance (SPR); and the second binding site has a binding affinity (KD) for the second antigen of 50 nM or more (eg, up to 1 mM) as determined by SPR.
   169. The CAL-cell or transplant of any one of paragraphs 161 to 168, wherein the first binding site has a binding affinity (KD) for the first antigen of 2 nM or less as determined by SPR and the second binding site has a binding affinity (KD) for the second antigen of 60 nM or more (eg, up to 1 mM) as determined by SPR.
   170. The CAL-cell or transplant of any one of paragraphs 161 to 169, wherein the first binding site has a binding affinity (KD) for the first antigen of 100 nM or less as determined by surface plasmon resonance (SPR).
   171. The CAL-cell or transplant of any one of paragraphs 161 to 170, wherein the first binding site has a binding off-rate for the first antigen of K_{off}=10⁻³ sec⁻¹ or less as determined by SPR.
   172. The CAL-cell or transplant of any one of paragraphs 161 to 172, wherein the second binding site has a binding affinity (KD) for the second antigen of 100 nM or less as determined by surface plasmon resonance (SPR).
   173. The CAL-cell or transplant of any one of paragraphs 161 to 172, wherein the binding affinity (KD) of the first binding site for the first antigen is higher than the affinity (KD) of the second binding site for the second antigen, wherein the affinity for the second antigen is less than 100 nM.
   174. The CAL-cell or transplant of any one of paragraphs 161 to 173, wherein the second binding site has a binding off-rate for the second antigen of K_{off}=10⁻³ sec⁻¹ or less as determined by SPR.
   175. The CAL-cell or transplant of any one of paragraphs 161 to 174, wherein each of the first and second antigen binding sites is selected from the group consisting of an scFv, Nanobody™, dAb, duocalin, DARpin, avimer, adnectin and fynomer.
   176. The CAL-cell or transplant of any one of paragraphs 161 to 175, wherein the size of the bridging agent is no more than 100 kDa.
   177. The CAL-cell or transplant of any one of paragraphs 161 to 176, wherein the size of the bridging agent is no more than 80 kDa (eg, no more than 50 or 55 kDa).
   178. The CAL-cell or transplant of any one of paragraphs 161 to 177 wherein the bridging agent is or comprises a BiTE™ antibody, bispecific-scFv, trispecific-scFv, Tandab™, dAb nanobody (eg, dimer or trimer), dAb multimer (eg, dimer or trimer), diabody, tetrabody or DART™.
   179. The CAL-cell or transplant of any one of paragraphs 161 to 178, wherein the bridging agent comprises a third antigen binding site.
   180. The CAL-cell or transplant of paragraph 179, wherein the third antigen is different from the first antigen.
   181. The CAL-cell or transplant of paragraph 179 or 180, wherein the third antigen is a tumour associated antigen (TAA), eg, a cell surface TAA comprised by the target cell.
   182. The CAL-cell or transplant of any one of paragraphs 161 to 180, wherein the first and/or third antigen is present more commonly on cancer cells than on normal cells.
   183. A CAL-immune cell, CAL-T cell, CAL-NK cell or CAL-TIL, wherein the CAL is as defined in any one of paragraphs 98 to 182.
   A CAR-immune cell, CAR-T cell, CAR-NK cell or CAR-TIL, wherein the CAR is as defined in any one of paragraphs 98 to 182 (with reference to any one of clauses VIII to XV).
   184. A population of CAL-immune cells, CAL-T cells, a population of CAL-NK cells or a population of CAL-TILs, wherein the cells are according to paragraph 183.
   A population of CAR-immune cells, CAR-T cells, a population of CAR-NK cells or a population of CAR-TILs, wherein the cells are according to paragraph 183.
   185. The cell or population of paragraph 183 or 184 for use in a method of treating or reducing the risk of a disease or condition in a human, wherein the method is according to any one of paragraphs 116 to 120.
   186. The cell or population of any one of paragraphs 98 to 185 comprised by a medical IV container, infusion device or syringe.
   187. A mammalian stem cell comprising a nucleotide sequence encoding an engineered transmembrane protein recited in any preceding paragraph.
   188. The cell of paragraph 187, wherein the engineered protein is a CAL.
   189. The cell of paragraph 187, wherein the engineered protein is a CAR. 190. The cell of any one of paragraphs 187 to 189, wherein the cell is pluripotent or multipotent.
The stem cell cannot develop into a human. In an embodiment, the stem cell cannot develop into a human embryo or zygote.
   191. The cell of any one of paragraphs 187 to 190, wherein the cell is a bone marrow stem cell.
   192. The cell of any one of paragraphs 187 to 191, wherein the cell is a haematopoietic stem cell.
   193. The cell of any one of paragraphs 187 to 192, wherein the cell is a non-human stem cell.
   194. The cell of any one of paragraphs 187 to 193, wherein the cell is ex vivo.
   195. A population of cells comprising a plurality of stem cells according to any one of paragraphs 187 to 194.
   196. The method of any one of paragraphs 1 to 97, wherein step (c) comprises administering to the human the bridging agent and the stem cell of any one of paragraphs 187 to 194, wherein the stem cell develops into said immune cell expressing the engineered transmembrane protein (eg, CAL or CAR), wherein the immune cell is combined with the target cell in the human.
   197. The method of paragraph 196, wherein the method comprises administering the population of paragraph 195, wherein said stem cells develop into a plurality of immune cells expressing the engineered transmembrane protein, wherein the immune cells are combined with target cells in the human.
   198. The cell or population of any one of paragraphs 187 to 195 for use in the method of paragraph 196 or 197 for treating or reducing the risk of a disease or condition in the human, eg, a cancer or autoimmune disease or condition.

Precision Immunotherapy: Domain Variation & Further Matching Aspects

It is recognized that individual humans differ in their sequence and recently several individuals have had their genomes sequenced, for instance James Watson and Craig Venter. Comparison of the genome sequence of individuals has revealed differences in their sequences in both coding and non-coding parts of the genome. Some of these variations in humans are significant and contribute to phenotypic differences between individuals. In extreme cases these will result in genetic disease. The 1000 Genomes Project has the objective of cataloguing sequences in the human genome, involving sequencing the genomes of a very large sampling of individuals from diverse art-recognized human ethnic populations.

Evidence is gathering that correlates intracellular signalling protein polymorphisms with various disease states that find application in the invention. For example, reference is made to: Genes Immun. 2015 March; 16(2):142-50. doi: 10.1038/gene.2014.73. Epub 2015 Jan. 8, “Genetic association of CD247 (CD3) with SLE in a large-scale multiethnic study”, Martins M et al; Rheumatology (Oxford). 2013 September; 52(9):1551-5. doi: 10.1093/rheumatology/ket119. Epub 2013 Mar. 22, “CD247 variants and single-nucleotide polymorphisms observed in systemic lupus erythematosus patients”, Takeuchi T & Suzuki K; “Polymorphisms in CD28, CTLA-4, CD80 and CD86 genes may influence the risk of multiple sclerosis and its age of onset”, Wagner M et al, J Neuroimmunol. 2015 Nov. 15; 288:79-86. doi: 10.1016/j.jneuroim.2015.09.004. Epub 2015 Sep. 18; “CTLA-4 and CD28 genes' polymorphisms and renal cell carcinoma susceptibility in the Polish population—a prospective study”, Tupikowski K et al, Tissue Antigens. 2015 November; 86(5):353-61. doi: 10.1111/tan.12671. Epub 2015 Sep. 25; and “CTLA-4, CD28, and ICOS gene polymorphism associations with non-small-cell lung cancer”, Karabon L, Hum Immunol. 2011 October; 72(10):947-54. doi: 10.1016/j.humimm.2011.05.010. Epub 2011 May 24.

Through the application of human genetic variation analysis and rationally-designed sequence selection the present invention provides for improved human patient therapy based on human variation in protein components of CARs and CALs. Importantly, the invention enables tailored medicines that address individual human patient genotypes or phenotypes.

The inventor's analysis of large numbers of naturally-occurring genomic human sequences reveals that there is significant variation across diverse human populations and provides for the ability for correlation between individual human patients and tailored medical approaches addressing the target. The technical applications of these findings, as per the present invention, thus contribute to better treatment and prophylaxis in humans and provides for patient benefit by enabling personalized medicines and therapies. This provides advantages of better prescribing, less wastage of medications and improved chances of drug efficacy in patients.

With this, the inventor realised that there is significant industrial and medical application for the invention in terms of guiding the choice of protein domains for CARs and CALs for administration to human patients for therapy and/or prophylaxis of diseases and conditions. In this way, the patient receives immunotherapy that is tailored to their needs—as determined by the patient's genetic or phenotypic makeup. Hand-in-hand with this, the invention provides for the genotyping and/or phenotyping of patients in connection with such treatment, thereby allowing a proper match of drug to patient. This increases the chances of medical efficacy, reduces the likelihood of inferior treatment using drugs that are not matched to the patient (eg, poor efficacy and/or side-effects) and avoids pharmaceutical mis-prescription and waste.

As described above, and as further explained in Example 1, an embodiment of the invention provides for matching of sequences used for engineering the transmembrane protein (eg, CAL or CAR) with the endogenous (ie, naturally-occurring) genotype of the recipient cell or human patient. In an aspect of this, the inventor has matched the engineered protein domain(s) to mirror natural variation found in human populations and found in the recipient human cell and patient genome. This is based partly on the realisation that naturally-tolerated amino acid variation (and corresponding non-synonymous SNPs) in humans will have co-evolved to work efficiently with the other components of the intracellular signalling machinery. As shown by the publications immediately above, mutation in signalling proteins can lead to undesirable effects, probably due in part to inferior signalling. The invention aims to match the engineered protein to more closely mirror endogenous proteins and genotypes of human cells and patients used in the invention.

As well as matching with intracellular signalling machinery, in another aspect the invention realises that the extracellular part of the engineered transmembrane protein will be exposed at the immune cell surface to the immune system of a recipient patient. Thus, the matching embodiment of the invention also realises the desirability of making the extracellular part of the transmembrane protein look as “self” as possible to the recipient human patient. Thus, in aspects one or more polymorphisms in the extracellular part (eg, in the second antigen of the CAL or in the hinge) is matched to polymorphism found naturally in the patient. In examples, the inventor has identified common individual polymorphisms or groups of polymorphisms that should be useful for a population of human cells and patients that match with such common polymorphisms.

As an extension of this, the invention identifies “universal frameworks” for domains of the transmembrane proteins of the invention. This is based on the identification of groups of residues in specific domains that are naturally permissive for variation in human populations; the invention has identified collections of such variations that each represent the most common polymorphism in humans and thus we believe will find utility in producing “universal CARs” and “universal CALs” that can be used with many human cells and human patients as they will match many natural polymorphisms in such cells and patients.

Thus, the invention provides the following specific aspects of this embodiment of the invention employing genomic and phenotypic matching.
1. A human immune cell comprising an engineered transmembrane protein, wherein the protein comprises
   A. an extracellular moiety comprising one or more ligand binding domains or one or more ligand domains;
   B. a transmembrane moiety; and
   C. an intracellular moiety comprising a first signaling domain (SD1);
      wherein
   D. the SD1 of the engineered protein is encoded in the cell by a first nucleotide sequence (S1) comprising a human single nucleotide polymorphism (SNP1) that encodes an amino acid residue (R1) of SD1;
   E. the genome of the cell comprises a second nucleotide sequence (S2) comprising SNP1 and encoding a second signaling domain (SD2), wherein the second signaling domain is (i) identical to SD1 and comprises R1 or (ii) a naturally-occurring variant of SD1 and comprises R1; and
   F. wherein S2 is an endogenous genomic sequence of the cell and SNP1 is a non-synonymous SNP.
      Additionally or alternatively, aspect 1 provides:—
      A human immune cell comprising an engineered transmembrane protein,

wherein the protein comprises
A. an extracellular moiety comprising a first antigen or ligand domain;
B. a transmembrane moiety; and
C. an intracellular moiety comprising a first signaling domain;
   wherein
D. the first antigen or ligand domain of the engineered protein is encoded in the cell by a first nucleotide sequence (S1) comprising a human single nucleotide polymorphism (SNP1) that encodes an amino acid residue (R1) of the antigen or ligand domain;
E. the genome of the cell comprises a second nucleotide sequence (S2) comprising SNP1 and encoding a second antigen or ligand domain, wherein the second antigen or ligand domain is (i) identical to the first antigen or ligand domain respectively and comprises R1 or (ii) a naturally-occurring variant of the first antigen or ligand domain respectively and comprises R1; and
F. wherein S2 is an endogenous genomic sequence of the cell and SNP1 is a non-synonymous SNP.
   Additionally or alternatively, aspect 1 provides:—
A human immune cell for use used in a method of treating or reducing the risk of a disease or condition (eg, as disclosed herein, eg, a cancer or autoimmune disease), wherein the method comprises administering the immune cell to a human patient, the immune cell comprising an engineered transmembrane protein, wherein the protein comprises
   A. an extracellular moiety comprising one or more ligand binding domains or one or more ligand domains;
   B. a transmembrane moiety; and
   C. an intracellular moiety comprising a first signaling domain (SD1);
wherein
   D. SD1 of the engineered protein is encoded in the cell by a first nucleotide sequence (S1) comprising a human single nucleotide polymorphism (SNP1) that encodes an amino acid residue (R1) of SD1;
   E. the genome of the human comprises a second nucleotide sequence (S2) comprising SNP1 and encoding a second signaling domain (SD2), wherein SD2 is (i) identical to SD1 and comprises R1 or (ii) a naturally-occurring variant of SD1 and comprises R1;
   F. wherein S2 is an endogenous genomic sequence of the human and SNP1 is a non-synonymous SNP; and
   G. wherein the human genome comprises S2 before said administration of the immune cell; and
   H. wherein the method treats or the risk of the disease or condition in the human.
      Additionally or alternatively, aspect 1 provides:—
      A human immune cell for use used in a method of treating or reducing the risk of a disease or condition (eg, as disclosed herein, eg, a cancer or autoimmune disease), wherein the method comprises administering the immune cell to a human patient, the immune cell comprising an engineered transmembrane protein,

wherein the protein comprises
A. an extracellular moiety comprising a first antigen or ligand domain;
B. a transmembrane moiety; and
C. an intracellular moiety comprising a first signaling domain;
   wherein
D. the first antigen or ligand domain of the engineered protein is encoded in the cell by a first nucleotide sequence (S1) comprising a human single nucleotide polymorphism (SNP1) that encodes an amino acid residue (R1) of the antigen or ligand domain;
E. the genome of the human comprises a second nucleotide sequence (S2) comprising SNP1 and encoding a second antigen or ligand domain, wherein the second antigen or ligand domain is (i) identical to the first antigen or ligand domain respectively and comprises R1 or (ii) a naturally-occurring variant of the first antigen or ligand domain respectively and comprises R1;
F. wherein S2 is an endogenous genomic sequence of the human and SNP1 is a non-synonymous SNP; and
G. wherein the human genome comprises S2 before said administration of the immune cell; and
H. wherein the method treats or the risk of the disease or condition in the human.
   In an example, S1 is at least 80, 90 or 95% identical to S2.

In an example, the extracellular moiety comprises an antigen binding domain. In an example, the transmembrane protein is a CAR, eg, any CAR disclosed herein.

In an example, the extracellular moiety comprises an antigen or a ligand (eg, a receptor ligand). In an example, the transmembrane protein is a CAL, eg, any CAL disclosed herein.

In an example, the immune cell is used in a method of treating or reducing the risk of a disease or condition (eg, as disclosed herein), wherein the method comprises administering the immune cell to a human patient, wherein the genome of the patient comprises S2 and/or SNP1 before said administration, wherein the disease or condition is treated or prevented in the human. In an example, the immune cell of the invention is for use in such a method.

As transmembrane proteins of the invention herein are “engineered”, this means that they are not naturally found in humans or human cells, or cells or mammals into which they are introduced.
2. The cell of aspect 1, wherein each said signaling domain is an intracellular domain selected from the group consisting of a CD3ζ (CD3-zeta) domain, CD3η (CD3-eta) domain, FcεRlγ domain, CD64 domain, CD16 domain, CD27 domain, CD28 domain, ICOS domain, OX40 (CD134) domain, CD40L domain and 4-1BB (CD137) domain.
3. The cell of any preceding aspect, wherein the first signaling domain is a CD3 intracellular domain selected from a CD3ζ (CD3-zeta) domain and a CD3η (CD3-eta) domain, and comprises at least 50 amino acid residues selected from the group consisting of V53, K54, F55, R57, S58, D60, Y64, Q65, Q68, L71, E74, L75, N76, L77, G78, R80, E81, Y83, L86, R89, G91, P94, E95, G98, K99, R102, Q107, G109, Y111, N112, E113, L114, Q115, K116, D117, K118, M119, E121, A122, Y123, 5124, E125, 1126, G127, G130, R134, G135, H138, D139, L141, Y142, Q143, G144, 5146, T147, T149, K150, D151, D154, H157, M158, Q159, L161 and P162 (position numbers correspond to positions of SEQ ID NO: 7).
4. The cell of aspect 3, wherein the first signaling domain is a CD3ζ (CD3-zeta) domain comprising all residues of said group.
5. The cell of aspect 3 or 4, wherein the first signaling domain comprises amino acid residues Y64, Y72 and Y83; Y64 and Y72; Y72 and Y83; Y111 and Y123; Y142 and Y153; Y64, Y72, Y83, Y111, Y123, Y142 and Y153; or Y64, Y72, Y111, Y123, Y142 and Y153; or Y72, Y83, Y111, Y123, Y142 and Y153 (position numbers correspond to positions of SEQ ID NO: 7).
6. The cell of any preceding aspect, wherein the first signaling domain is a CD3 intracellular domain selected from a CD3ζ (CD3-zeta) domain and a CD3η (CD3-eta) domain, and comprises SEQ ID NO: 9.
   This is a universal framework according to the invention (explained above and further in Example 1).
7. A human immune cell comprising an engineered transmembrane protein,
   wherein the protein comprises
      A. an extracellular moiety comprising one or more ligand binding domains or one or more ligand domains;
      B. a transmembrane moiety; and
      C. an intracellular moiety comprising a first signaling domain;
      D. wherein the first signaling domain is a CD3 intracellular domain selected from a CD3 (CD3-zeta) domain and a CD3η (CD3-eta) domain, and comprises at least 50 amino acid residues selected from the group consisting of V53, K54, F55, R57, S58, D60, Y64, Q65, Q68, L71, E74, L75, N76, L77, G78, R80, E81, Y83, L86, R89, G91, P94, E95, G98, K99, R102, Q107, G109, Y111, N112, E113, L114, Q115, K116, D117, K118, M119, E121, A122, Y123, 5124, E125, 1126, G127, G130, R134, G135, H138, D139, L141, Y142, Q143, G144, S146, T147, T149, K150, D151, D154, H157, M158, Q159, L161 and P162 (position numbers correspond to positions of SEQ ID NO: 1); and
      E. wherein the genome of the cell comprises an endogenous nucleotide sequence encoding a second signaling domain, wherein the second domain is a CD3ζ (CD3-zeta) domain or a CD3η (CD3-eta) domain comprising at least 40 (eg, 45 or all) of said selected residues.

This is based on the identification by the inventor of groups of residues in CD3 and CD3η domains that are naturally permissive for variation in human populations; the invention has identified collections of such variations that each represent the most common polymorphism in humans and thus we believe can be used with many human cells and human patients as they will match many natural polymorphisms in such cells and patients.

Additionally or alternatively, aspect 7 provides:—

A human immune cell for use used in a method of treating or reducing the risk of a disease or condition (eg, as disclosed herein, eg, a cancer or autoimmune disease), wherein the method comprises administering the immune cell to a human patient, the immune cell comprising an engineered transmembrane protein, wherein the protein comprises
A. an extracellular moiety comprising a first antigen or ligand domain;
B. a transmembrane moiety; and
C. an intracellular moiety comprising a first signaling domain;
   wherein
D. wherein the first signaling domain is a CD3 intracellular domain selected from a CD3 (CD3-zeta) domain and a CD3η (CD3-eta) domain, and comprises at least 50 amino acid residues selected from the group consisting of V53, K54, F55, R57, S58, D60, Y64, Q65, Q68, L71, E74, L75, N76, L77, G78, R80, E81, Y83, L86, R89, G91, P94, E95, G98, K99, R102, Q107, G109, Y111, N112, E113, L114, Q115, K116, D117, K118, M119, E121, A122, Y123, 5124, E125, 1126, G127, G130, R134, G135, H138, D139, L141, Y142, Q143, G144, S146, T147, T149, K150, D151, D154, H157, M158, Q159, L161 and P162 (position numbers correspond to positions of SEQ ID NO: 1); and
E. wherein the genome of the human comprises an endogenous nucleotide sequence encoding a second signaling domain, wherein the second domain is a CD3ζ (CD3-zeta) domain or a CD3η (CD3-eta) domain comprising at least 40 (eg, 45 or all) of said selected residues;
F. wherein the method treats or the risk of the disease or condition in the human.
8. The cell of aspect 7, wherein the first signaling domain comprises amino acid residues Y64, Y72 and Y83; Y64 and Y72; Y72 and Y83; Y111 and Y123; Y142 and Y153; Y64, Y72, Y83, Y111, Y123, Y142 and Y153; or Y64, Y72, Y111, Y123, Y142 and Y153; or Y72, Y83, Y111, Y123, Y142 and Y153 (position numbers correspond to positions of SEQ ID NO:7).
9. The cell of aspect 7 or 8, wherein the first signaling domain is a CD3ζ (CD3-zeta) domain comprising SEQ ID NO: 9; and the second domain is a CD3ζ (CD3-zeta) domain comprising SEQ ID NO: 9.
10. The cell of any one of aspects 7 to 9, wherein the first and second signaling domains are CD3 (CD3-zeta) domains and optionally each domain comprises said at least 50 selected residues.
11. The cell of any preceding aspect, wherein each of the first and second signaling domains comprises an immunoreceptor tyrosine-based activation motif (ITAM) comprising R1.
12. The cell of aspect 11, wherein the ITAMs of the signaling domains are identical.
13. The cell of any preceding aspect, wherein the first and second signaling domains are identical.
14. The cell of any preceding aspect, wherein the first signaling domain is human.
15. The cell of any preceding aspect, wherein the intracellular moiety comprises a further intracellular signaling domain (third signaling domain, SD3) that is encoded in the cell by a third nucleotide sequence (S3), S3 comprising a human single nucleotide polymorphism (SNP2) that encodes an amino acid residue (R2) of SD3; wherein the genome of the cell comprises a fourth nucleotide sequence (S4) encoding a fourth signaling domain (SD4), wherein SD4 is (iii) identical to SD3 and comprises R2 or (iv) a variant of SD3 and comprises R2; and wherein S4 is an endogenous genomic sequence of the cell and SNP2 is a non-synonymous SNP.
   In an additional or alternative aspect, the intracellular moiety comprises a further intracellular signaling domain (third signaling domain, SD3) that is encoded in the human genome by a third nucleotide sequence (S3), S3 comprising a human single nucleotide polymorphism (SNP2) that encodes an amino acid residue (R2) of 5D3; wherein the genome of the human comprises a fourth nucleotide sequence (S4) encoding a fourth signaling domain (SD4), wherein SD4 is (iii) identical to SD3 and comprises R2 or (iv) a variant of SD3 and comprises R2; and wherein S4 is an endogenous genomic sequence of the human and SNP2 is a non-synonymous SNP.
16. The cell of aspect 15, wherein SD1 and SD3 domains are different.
17. The cell of aspect 15 or 16, wherein each of SD3 and SD4 comprises an ITAM comprising R2.
18. The cell of aspect 17, wherein the ITAMs of the SD3 and SD4 are identical.
19. The cell of any one of aspects 15 to 18, wherein SD3 and SD4 are identical.
20. The cell of any one of aspects 15 to 19, wherein SD3 is human.
21. The cell of any one of aspects 15 to 20, wherein each of SD3 and SD4 is a CD3ζ (CD3-zeta) domain, CD3η (CD3-eta) domain, FcεRlγ domain, CD64 domain, CD16 domain, CD27 domain, CD28 domain, ICOS domain, OX40 domain, CD40 domain or 4-1BB domain.
22. The cell of any one of aspects 15 to 21, wherein SD1 is a CD3 intracellular domain selected from a CD3ζ (CD3-zeta) domain and a CD3η (CD3-eta) domain, and the third domain is a FcεRlγ domain, CD64 domain, CD16 domain, CD27 domain, CD28 domain, ICOS domain, OX40 domain, CD40 domain or 4-1BB domain.
23. The cell of any preceding aspect, wherein SD1 is a CD3 intracellular domain selected from a CD3 (CD3-zeta) domain and a CD3η (CD3-eta) domain and is the C-terminal domain of the transmembrane protein.
24. The cell of any preceding aspect, wherein SD1 is a CD3 intracellular domain selected from a CD3 (CD3-zeta) domain and a CD3η (CD3-eta) domain, and comprises one, two or three amino acid motifs selected from (a) SEQ ID NO: 10 optionally with up to 10, 9, 8, 7, 6 or five (eg, up to 5) amino acid differences, but wherein the tyrosines are conserved; (b) SEQ ID NO: 11 optionally with up to 10, 9, 8, 7, 6 or five (eg, up to 5) amino acid differences, but wherein the tyrosines are conserved; and (c)) SEQ ID NO: 12 optionally with up to 10, 9, 8, 7, 6 or five (eg, up to 5) amino acid differences, but wherein the tyrosines are conserved.
25. The cell of aspect 24, wherein SD1 comprises motif (a) and wherein the motif differs from NO: 10 by a change of up to five residues selected from the group consisting of A61, P62, A63, Q66, G67, N69, Q70, N73, R79, E82, D84, V85, D87 and K88.
26. The cell of aspect 25, wherein the changes are selected from the group consisting of A61V, A61P, P62S, P62A, A63P, Q66H, G67S, N69T, Q70Y, Q70L, Q70P, Q70W, N73Y, R79L, R79G, E82K, D84G, D84A, D84Y, V851, D87G and K88R.
27. The cell of any one of aspects 24 to 26, wherein SD1 comprises motif (b) and wherein the motif differs from SEQ ID NO: 11 by a change of up to five residues selected from the group consisting of P100, Q101, R103, K104, N105, P106, E108, L110, A120, A122 and M128.
28. The cell of aspect 27, wherein the changes are selected from the group consisting of P100L, Q101L, Q101P, R103K, K104E, N105K, P106R, E108A, L110Q, A120V, A122V and M128T.
29. The cell of any one of aspects 24 to 28, wherein SD1 comprises motif (c) and wherein the motif differs from SEQ ID NO: 12 by a change of up to five residues selected from the group consisting of E131, R132, R133, K136, G137, G140, L145, A148, T152, A155, L156.
30. The cell of aspect 29, wherein the changes are selected from the group consisting E131K, R132H, R132C, R133Q, R133W, K136N, G137E, G140D, L145F, A148D, T1521, A155T, L156P.
31. The cell of any preceding aspect, wherein SD1 is a CD3 intracellular domain selected from a CD3 (CD3-zeta) domain and a CD3η (CD3-eta) domain, the first signaling domain comprising a plurality (eg, 3) ITAMs, wherein the second signaling domain comprises identical corresponding ITAMs.
32. The cell of any preceding aspect, wherein SD1 is a CD3 intracellular domain selected from a CD3 (CD3-zeta) domain and a CD3η (CD3-eta) domain, and comprises one, more or all amino acid residues selected from the group consisting of S58, Y64, Y72, Y83, Y111, Y123, Y142 and Y153 (position numbers correspond to positions of SEQ ID NO: 7.
33. The cell of aspect 32, wherein SD1 comprises one or both of C72 and N153.
34. The cell of any preceding aspect, wherein SD1 is a CD3 intracellular domain selected from a CD3 (CD3-zeta) domain and a CD3η (CD3-eta) domain, and comprises a residue (said R1) selected from the group consisting of R52, S56, A59, A61, P62, A63, Q66, G67, N69, Q70, Y72, N73, R79, E82, D84, V85, D87, K88, R90, R92, D93, M96, G97, P100, Q101, R103, K104, N105, P106, E108, L110, A120, A122, M128, K129, E131, R132, R133, K136, G137, G140, L145, A148, T152, Y153, A155, L156, A160, P163 and R164 (position numbers correspond to positions of SEQ ID NO: 7).
35. The cell of any preceding aspect, wherein SD1 is a CD3 intracellular domain selected from a CD3 (CD3-zeta) domain and a CD3η (CD3-eta) domain, and comprises a residue (said R1) that is Y72 or Y153 (position numbers correspond to positions of SEQ ID NO: 7).
36. The cell of any preceding aspect, wherein SD1 is a CD3 intracellular domain selected from a CD3 (CD3-zeta) domain and a CD3η (CD3-eta) domain, and comprises a residue (said R1) encoded by a SNP (SNP1) selected from the group consisting of r5368651001, r5372651048, r5767112686, r5765877580, r5751145956, r5772867144, r555893506, r5761710510, r5776601547, rs768607376, rs193922741, rs193922740, rs193922739, rs780188126, rs772128174, rs757978223, rs779397562, rs749926653, rs181746205, rs181746205, rs753572867, rs371709798, rs145407267, rs143180729, rs148513413, rs367690333, rs144963570, rs367690333, rs144963570, rs770320255, rs139926301, rs56297636, rs760895755, rs112890541, rs370910340, rs145505909, rs754935006, rs751583971, rs766541481, rs763074967, rs745871212, rs372665461, rs764185491, rs756340039, rs773572491, rs201594815, rs781510519, rs147527561, rs751981677, rs763532939, rs753278244, rs771873949, rs186004179, rs186004179, rs762773775, rs748158220, rs776703680, rs561262982, rs758846009, rs746262183, rs376046446, rs201937405 and rs752198795.
37. The cell of any preceding aspect, wherein SD1 is a CD3 intracellular domain selected from a CD3 (CD3-zeta) domain and a CD3η (CD3-eta) domain, and comprises a residue (said R1) selected from the group consisting of R52, S56, A59, A61, P62, A63, Q66, G67, N69, Q70, Y72, N73, R79, E82, D84, V85, D87, K88, R90, R92, D93, M96, G97, P100, Q101, R103, K104, N105, P106, E108, L110, A120, A122, M128, K129, E131, R132, R133, K136, G137, G140, L145, A148, T152, Y153, A155, L156, A160, P163 and R164 (position numbers correspond to positions of SEQ ID NO: 7); and optionally wherein SD2 also comprises said selected residue (R1).
38. The cell of any preceding aspect, wherein SD1 or SD3 is a CD28 intracellular domain comprising at least 13 amino acid residues selected from the group consisting of R180, 5181, K182, R183, 5184, R185, L186, D190, Y191, N193, P196, P199, T202, K204, Q207, F215, A217 and Y218 (position numbers correspond to positions of SEQ ID NO: 13).
39. The cell of aspect 38, wherein the CD28 domain comprises all residues of said group.
40. The cell of aspect 38 or 39, wherein the CD28 domain comprises amino acid residues Y191 and Y209 (position numbers correspond to positions of SEQ ID NO: 13).
41. The cell of any one of aspects 38 to 40, wherein the CD28 domain comprises a YMNM motif (corresponding to Y191-M192-N193-M194 of SEQ ID NO: 13) and/or a PYAP motif (corresponding to P208-Y209-A210-P211 of SEQ ID NO: 13).
42. The cell of any preceding aspect, wherein the CD domain comprises SEQ ID NO: 15.
43. A human immune cell comprising an engineered transmembrane protein,
   wherein the protein comprises
      A. an extracellular moiety comprising a first antigen or ligand domain;
      B. a transmembrane moiety; and
      C. an intracellular moiety comprising a first signaling domain (SD1);
      D. wherein SD1 is a CD28 intracellular domain comprising at least 13, 14, 15, 16, 17 or 18 amino acid residues selected from the group consisting of R180, 5181, K182, R183, 5184, R185, L186, D190, Y191, N193, P196, P199, T202, K204, Q207, F215, A217 and Y218 (position numbers correspond to positions of SEQ ID NO: 13); and
      E. wherein the genome of the cell comprises an endogenous nucleotide sequence encoding a second signaling domain (SD2), wherein SD2 is a CD28 intracellular domain comprising at least 10 (or 11, 12 or 13) of said selected residues.

Additionally or alternatively, aspect 7 provides:—

A human immune cell for use used in a method of treating or reducing the risk of a disease or condition (eg, as disclosed herein, eg, a cancer or autoimmune disease), wherein the method comprises administering the immune cell to a human patient, the immune cell comprising an engineered transmembrane protein, wherein the protein comprises
A. an extracellular moiety comprising a first antigen or ligand domain;
B. a transmembrane moiety; and
C. an intracellular moiety comprising a first signaling domain (SD1);
   wherein
D. wherein SD1 is a CD28 intracellular domain comprising at least 13, 14, 15, 16, 17 or 18 amino acid residues selected from the group consisting of R180, 5181, K182, R183, 5184, R185, L186, D190, Y191, N193, P196, P199, T202, K204, Q207, F215, A217 and Y218 (position numbers correspond to positions of SEQ ID NO: 13);
E. wherein the genome of the human comprises an endogenous nucleotide sequence encoding a second signaling domain (SD2), wherein SD2 is a CD28 intracellular domain comprising at least 10 (or 11, 12 or 13) of said selected residues; and
F. wherein the method treats or the risk of the disease or condition in the human.
44. The cell of aspect 43, wherein SD1 comprises amino acid residues Y191 and Y209 (position numbers correspond to positions of SEQ ID NO: 13).
45. The cell of aspect 43 or 44, wherein SD1 comprises SEQ ID NO: 15; and the second domain is a CD28 domain comprising SEQ ID NO: 15.
46. The cell of any one of aspects 43, 44 or 45, wherein SD1 and SD2 each domain comprises said at least 13 selected residues.
47. The cell of any one of aspects 43 to 46, wherein the intracellular moiety comprises a further signaling domain (third signaling domain, SD3) that is encoded in the cell by a third nucleotide sequence (S3), S3 comprising a human single nucleotide polymorphism (SNP2) that encodes an amino acid residue (R2) of SD3; wherein the genome of the cell or human comprises a fourth nucleotide sequence (S4) encoding a fourth signaling domain (SD4), wherein SD4 is (iii) identical to SD3 and comprises R2 or (iv) a variant of SD3 and comprises R2; and wherein SD4 is an endogenous genomic sequence of the cell or human respectively.
48. The cell of aspect 47, wherein SD1 and SD3 domains are different.
49. The cell of aspect 47 or 48, wherein SD3 and SD4 are identical.
50. The cell of any one of aspects 47 to 49, wherein SD3 is human.
51. The cell of any one of aspects 47 to 50, wherein each of SD3 and SD4 is an intracellular domain selected from the group consisting of a CD3ζ (CD3-zeta) domain, CD3η (CD3-eta) domain, FcεRlγ domain, CD64 domain, CD16 domain, CD27 domain, ICOS domain, OX40 domain, CD40 domain and 4-1BB domain.
52. The cell of any one of aspects 47 to 51, wherein SD1 is the C-terminal domain of the receptor or the N-terminal-most intracellular domain of the receptor.
53. The cell of any preceding aspect, wherein SD1 or SD3 domain is a CD28 intracellular domain comprising a residue (said R1 or R2) selected from the group consisting of L187, H188, 5189, M192, M194, T195, R197, R198, G200, P201, R203, H205, Y206, P208, Y209, A210, P211, P212, R213, D214, A216, R219 and 5220 (position numbers correspond to positions of SEQ ID NO: 13).
54. The cell of any preceding aspect, wherein SD1 or SD3 is a CD28 intracellular domain comprising a residue (said R1 or R2) that is M192, M194, P208, Y209, A210 or P211 (position numbers correspond to positions of SEQ ID NO: 13).
55. The cell of any one of aspects, wherein SD1 or SD3 is a CD28 intracellular domain, and comprises a residue (said R1 or R2) encoded by a SNP (SNP1 or SNP2) selected from the group consisting of r5139881881, r5139881881, r5751945323, rs753396357, r5754453810, r5200221759, rs562969933, r5765515314, r5145761335, r5199647272, r5200751829, r5201547332, rs200642723, rs367908475, rs199549636, rs199549636, rs749688881, rs769098383, rs572738990, rs200606770, rs371850110, rs201773411, rs762I44222, rs770610915, r5199777674, r5201909740, r5200016310, r5200936737, r5201598596 and rs762747357.
56. The cell of any preceding aspect when dependent on aspect 15 and/or 47, wherein SD3 and SD4 are human 4-1BB domains and R2 is selected from the group consisting of R215, Q215, W215, R217, G217, K218, N218, Y222, C222, P227, 5227, M229, 1229, V232, A232, Q236, H236, D239, C241, Y241, R244, Q244, E247, G247, E250, G250, G252, E252, V252, R252, C253 and 5253 (position numbers correspond to positions of SEQ ID NO: 16).
57. The cell of aspect 56, wherein SD3 comprises at least 10, 11, 12, 13, 14 or all of the residues selected from the group consisting of R215, R217, K218, Y222, P227, M229, V232, Q236, D239, C241, R244, E247, E250, G252 and C253 (position numbers correspond to positions of SEQ ID NO: 16).
58. The cell of any preceding aspect when dependent on aspect 15 and/or 47, wherein SD3 and SD4 are human 4-1BB domains, wherein SD3 comprises Q236 and D239 (position numbers correspond to positions of SEQ ID NO: 16).
   These are conserved TRAF binding residues, as explained in Example 1.
59. The cell of any preceding aspect, when dependent on aspect 15 and/or 47, wherein SD3 comprises SEQ ID NO: 18.
   This is a universal 4-1BB framework of the invention, which retains conserved, naturally-occurring residues and the TRAF binding motifs.
60. A human immune cell comprising an engineered transmembrane protein, wherein the protein comprises
   F. an extracellular moiety comprising a first antigen or ligand domain;
   G. a transmembrane moiety; and
   H. an intracellular moiety comprising a first signaling domain (SD1);
   I. wherein SD1 is a 4-1BB intracellular domain comprising at least 10, 11, 12, 13, 14 or all of the residues selected from the group consisting of R215, R217, K218, Y222, P227, M229, V232, Q236, D239, C241, R244, E247, E250, G252 and C253 (position numbers correspond to positions of SEQ ID NO: 16); and
   J. wherein the genome of the cell comprises an endogenous nucleotide sequence encoding a second signaling domain (SD2), wherein SD2 is a 4-1BB intracellular domain comprising at least 8 (or 9 or 10) of said selected residues.

Additionally or alternatively, aspect 7 provides:—

A human immune cell for use used in a method of treating or reducing the risk of a disease or condition (eg, as disclosed herein, eg, a cancer or autoimmune disease), wherein the method comprises administering the immune cell to a human patient, the immune cell comprising an engineered transmembrane protein, wherein the protein comprises
G. an extracellular moiety comprising a first antigen or ligand domain;
H. a transmembrane moiety; and
I. an intracellular moiety comprising a first signaling domain (SD1);
   wherein
J. wherein SD1 is a 4-1BB intracellular domain comprising at least 10, 11, 12, 13, 14 or all of the residues selected from the group consisting of R215, R217, K218, Y222, P227, M229, V232, Q236, D239, C241, R244, E247, E250, G252 and C253 (position numbers correspond to positions of SEQ ID NO: 16); and
K. wherein the genome of the human comprises an endogenous nucleotide sequence encoding a second signaling domain (SD2), wherein SD2 is a 4-1BB intracellular domain comprising at least 8 (or 9 or 10) of said selected residues.
L. wherein the method treats or the risk of the disease or condition in the human.
61. The cell of aspect 60, wherein SD1 comprises amino acid residues Q236, D239, E247 and E250 (position numbers correspond to positions of SEQ ID NO: 16).
62. The cell of aspect 60 or 61, wherein SD1 comprises SEQ ID NO: 18; and the second domain is a 4-1BB domain comprising SEQ ID NO: 18.
63. The cell of any one of aspects 60, 61 or 62, wherein SD1 and SD2 each domain comprises said at least 13 selected residues.
64. The cell of any one of aspects 60 to 63, wherein the intracellular moiety comprises a further signaling domain (third signaling domain, SD3) that is encoded in the cell by a third nucleotide sequence (S3), S3 comprising a human single nucleotide polymorphism (SNP2) that encodes an amino acid residue (R2) of 5D3; wherein the genome of the cell or human comprises a fourth nucleotide sequence (S4) encoding a fourth signaling domain (SD4), wherein SD4 is (iii) identical to SD3 and comprises R2 or (iv) a variant of SD3 and comprises R2; and wherein SD4 is an endogenous genomic sequence of the cell or human respectively.
65. The cell of aspect 64, wherein SD1 and SD3 domains are different.
66. The cell of aspect 64 or 65, wherein SD3 and SD4 are identical.
67. The cell of any one of aspects 64 to 66, wherein SD3 is human.
68. The cell of any one of aspects 64 to 67, wherein each of SD3 and SD4 is an intracellular domain selected from the group consisting of a CD3ζ (CD3-zeta) domain, CD3η (CD3-eta) domain, FcεRlγ domain, CD64 domain, CD16 domain, CD27 domain, ICOS domain, OX40 domain, CD40 domain and CD28 domain.
69. The cell of any one of aspects 60 to 68, wherein SD1 is the C-terminal domain of the receptor or the N-terminal-most intracellular domain of the receptor.
70. The cell of any preceding aspect, wherein SD1 or SD3 domain is a 4-1BB intracellular domain comprising a residue (said R1 or R2) selected from the group consisting of R215, R217, K218, Y222, P227, M229, V232, Q236, D239, C241, R244, E247, E250, G252 and C253 (position numbers correspond to positions of SEQ ID NO: 16).
71. The cell of any preceding aspect, wherein SD1 or SD3 is a 4-1BB intracellular domain comprising a residue (said R1 or R2) that is Q236, D239, E247 or E250 (position numbers correspond to positions of SEQ ID NO: 16).
72. The cell of any one of aspects, wherein SD1 or SD3 is a 4-1BB intracellular domain, and comprises a residue (said R1 or R2) encoded by a SNP (SNP1 or SNP2) selected from the group consisting of r5753016242, r5143524950, r5780812476, rs755927735, r5144908104, rs533883433, r5367584804, r5141498457, r5751542955, r5764017912, r5752191416, r5554909019, r5759184548, r5776878260, r5113310001, r5113310001, r5761088691 and r5772691718.
73. The cell of any preceding aspect, wherein the extracellular moiety comprises an antigen-binding site comprising an antibody VH domain, wherein the VH domain is derived from the recombination of a human VH gene segment with a DH and a JH gene segments, wherein the VH gene segment comprises a SNP (SNP3) that is comprised by the genome of the cell or human.
74. The cell of any preceding aspect, wherein the extracellular moiety comprises an antigen-binding site comprising an antibody VL domain, wherein the VL domain is derived from the recombination of a human VL gene segment and a JL gene segment, wherein the VL gene segment comprises a SNP (SNP4) that is comprised by the genome of the cell or human.
75. The cell of any one of aspects 1 to 73, wherein the extracellular moiety comprises an antigen-binding site comprising a TCR Vα domain, wherein the Vα domain is derived from the recombination of a human Vα gene segment with a Jα gene segment, wherein the Vα gene segment comprises a SNP (SNPS) that is comprised by the genome of the cell or human.
76. The cell of any one of aspects 1 to 73 and 74, wherein the extracellular moiety comprises an antigen-binding site comprising a TCR VB domain, wherein the Vβ domain is derived from the recombination of a human Vβ gene segment with Dβ and Jβ gene segments, wherein the Vβ gene segment comprises a SNP (SNP6) that is comprised by the genome of the cell or human.
77. The cell of any preceding aspect, wherein the extracellular moiety comprises a TCR Ca sequence that is encoded by a Ca gene segment sequence, wherein the Ca gene segment comprises a SNP (SNP7) that is comprised by the genome of the cell or human, wherein SNP7 encodes a Ca extracellular amino acid residue (R3) and R3 is comprised by the Ca sequence of the extracellular moiety.
78. The cell of any preceding aspect, wherein the extracellular moiety comprises a TCR Cβ sequence that is encoded by a Cβ gene segment sequence, wherein the Cβ gene segment comprises a SNP (SNP8) that is comprised by the genome of the cell or human, wherein SNP8 encodes a Cβ extracellular amino acid residue (R4) and R4 is comprised by the Cβ| sequence of the extracellular moiety.
79. The cell of any preceding aspect, wherein the cell is a T-cell (eg, CD8⁺ T-cell, eg, an activated T-cell), NK cell, tumour-infiltrating lymphocyte (TIL, eg, a pre-REP TIL), stem cell, memory stem cell, bone marrow cell, bone marrow stem cell, haematopoietic stem cell, memory T-cell, T_{SCM}, T_{CM} or T_{EM}.
80. The cell of any preceding aspect, wherein the cell has been engineered for enhanced signaling, wherein the signaling is selected from CD28, 4-1BB, OX40, ICOS and CD40 signalling.
81. A population of immune cells comprising a plurality of cells according to any preceding aspect.
82. A cell or population of any preceding aspect for treating a cancer, inflammatory disease, autoimmune disease or a viral infection in a human.
   In an embodiment, the cancer, disease or condition is any cancer, disease or condition disclosed herein.
83. The cell or population of aspect 82, wherein the cancer is a cancer of T-cell or B-cell origin, eg, lymphoblastic leukemia, ALL (eg, T-ALL), CLL (eg, B-cell chronic lymphocytic leukemia) or non-Hodgkin's lymphoma.
84. The cell or population of aspect 82 or 89, wherein each said cell is an autologous cell (eg, T-cell) of said human or is a progeny of such an autologous cell.
85. The cell or population of aspect 84, wherein each autologous cell is derived from a blood or tumor sample of the human and activated and expanded in vitro.
86. A method of producing a cell according to any preceding aspect, the method comprising
   i. obtaining a human immune cell (eg, a memory T-cell, NK cell, bone marrow cell, stem cell or TIL);
   ii. obtaining an expressible nucleotide sequence encoding said engineered transmembrane protein, wherein the protein comprises said residue (R1) encoded by a SNP (SNP1) that is comprised by the genome of the cell; and
   iii. Introducing the nucleotide sequence into the cell for expression of the receptor.
87. The method of aspect 86, further comprising culturing, differentiating and/or activating the cell produced by step (iii), thereby producing a population of cells expressing the receptor.
88. The method of aspect 86 or 87, wherein the cell of step (i) is obtained from a human suffering from a cancer, inflammatory disease, autoimmune disease or a viral infection.
89. A method of treating a cancer, inflammatory disease, autoimmune disease or a viral infection in a human, the method comprising administering a cell or population of any preceding aspect or produced by the method of aspect 87 or 88 to the human, wherein cell killing is achieved and the cancer, disease or condition is treated.
   In an example, the severity or progression of the cancer, disease or infection in the human is reduced.
90. The method of aspect 89, wherein the administered cell is a progeny of a cell that was obtained from the human and used in step (i) of the method of aspect 86, 87 or 88.
91. A cell or population of any preceding aspect for use in a method of aspect 89 or 90 to treat or reduce the risk or progression of a cancer, inflammatory disease, autoimmune disease or a viral infection in a human.
92. A medical V bag or injection device comprising a cell or population of any one of aspects 1 to 85 and 91.
93. A mammalian stem cell comprising a nucleotide sequence encoding an engineered transmembrane protein recited in any preceding aspect.
94. The cell of aspect 93, wherein the engineered protein is a CAL.
95. The cell of aspect 93, wherein the engineered protein is a CAR.
96. The cell of any one of aspects 93 to 95, wherein the cell is pluripotent or multipotent.
   The stem cell cannot develop into a human. In an embodiment, the stem cell cannot develop into a human embryo or zygote.
97. The cell of any one of aspects 93 to 96, wherein the cell is a bone marrow stem cell.
98. The cell of any one of aspects 93 to 97, wherein the cell is a haematopoietic stem cell.
99. The cell of any one of aspects 93 to 98, wherein the cell is a non-human stem cell.
100. The cell of any one of aspects 93 to 99, wherein the cell is ex vivo.
101. A population of cells comprising a plurality of stem cells according to any one of aspects 93 to 100.
102. The method of any preceding aspects, wherein the method comprising the immune cell and a bridging agent to the human to treat or prevent a disease or condition in the human.
103. The method of aspect 102 comprising administering the stem cell of any one of aspects 93 to 100 to the human, wherein the stem cell develops into said immune cell expressing the engineered transmembrane protein (eg, CAL or CAR), wherein the immune cell is combined with a target cell in the human, wherein the engineered immune cell is activated and the target cell killed.
104. The method of aspect 103, wherein the method comprises administering the population of paragraph 101, wherein said stem cells develop into a plurality of immune cells expressing the engineered transmembrane protein, wherein the immune cells are combined with target cells in the human and engineered immune cells are activated and target cells killed, whereby the disease or condition is treated or prevented.
105. The cell or population of any one of aspects 93 to 101 for use in the method of aspect 103 or 104 for treating or reducing the risk of a disease or condition in the human, eg, a cancer or autoimmune disease or condition.

As set out in the Examples, the inventor has designed universal intracellular signalling domain frameworks: SEQ ID NOs: 9, 15 and 18. To this end, the invention also provides the following aspects:—
1. A CAL-immune or CAR-immune cell whose genome comprises one more or all of (i) to (iii):—
   (i) an endogenous nucleotide sequence encoding a CD3 zeta intracellular domain comprising SEQ ID NO: 9; and a nucleotide sequence encoding a CD3 zeta intracellular domain of the CAL or CAR which comprises SEQ ID NO: 9;
   (ii) an endogenous nucleotide sequence encoding a CD28 intracellular domain comprising SEQ ID NO: 15; and a nucleotide sequence encoding a CD28 intracellular domain of the CAL or CAR which comprises SEQ ID NO: 15; and/or
   (iii) an endogenous nucleotide sequence encoding a 4-1BB intracellular domain comprising SEQ ID NO: 18; and a nucleotide sequence encoding a 4-1BB intracellular domain of the CAL or CAR which comprises SEQ ID NO: 18.
2. An autologous or allogeneic cell transplant for administration to a human patient to treat or prevent a disease or condition (eg, a cancer), the transplant comprising a plurality of CAL-immune or CAR-immune cells, wherein the cells are progeny of one or more ancestor cells obtained from a human donor, wherein the genome of each said cell of the transplant comprises one more or all of (i) to (iii):—
   (i) a nucleotide sequence encoding a CD3 zeta intracellular domain of the CAL or CAR which comprises SEQ ID NO: 9, wherein the genome(s) of the ancestor cell(s) comprise an endogenous nucleotide sequence encoding a CD3 zeta intracellular domain which comprises SEQ ID NO: 9;
   (ii) a nucleotide sequence encoding a CD28 intracellular domain of the CAL or CAR which comprises SEQ ID NO: 15, wherein the genome(s) of the ancestor cell(s) comprise an endogenous nucleotide sequence encoding a CD28 intracellular domain which comprises SEQ ID NO: 15; or
   (iii) a nucleotide sequence encoding a 4-1BB intracellular domain of the CAL or CAR which comprises SEQ ID NO: 18, wherein the genome(s) of the ancestor cell(s) comprise an endogenous nucleotide sequence encoding a 4-1BB intracellular domain which comprises SEQ ID NO: 18.
3. The transplant of aspect 2, wherein each said cell of the transplant comprises said endogenous sequence(s).
   In an example, the CAL or CAR comprises SEQ ID NO: 9 and said transplant comprises an endogenous CD3 zeta intracellular domain sequence encoding SEQ ID NO: 9; and/or the CAL or CAR comprises SEQ ID NO: 15 and said transplant comprises an endogeous CD28 intracellular domain sequence encoding SEQ ID NO: 15; and/or the CAL or CAR comprises SEQ ID NO: 18 and said transplant comprises an endogenous 4-1BB intracellular domain sequence encoding SEQ ID NO: 18.
   In an alternative, one or more of such endogenous sequences may be knocked out (ie, rendered non functional or non expressible) in said transplant cells or immune cell(s). Yet, the cells will still express signaling machinery that in the donor functions with intracellular domains of the CAR or CAL comprising one or more of SEQ ID NOs: 9, 15 and 18. Such knock-outs may be of use to focus the signaling to the CAR or CAL of the cell and not the endogenous signalling domains.
4. The cell or transplant of any one of aspects 1 to 3, for use in a method of treating or preventing a disease or condition (eg, a cancer) in a human patient, wherein the germline genome of the patient comprises said intracellular domain endogenous sequence(s), optionally wherein the germline genome encodes CD3 zeta, CD28 and 4-1BB intracellular domains respectively comprising SEQ ID NOs: 9, 15 and 18.
   In an example, the CAL or CAR comprises SEQ ID NO: 9 and said germline genome comprises an endogenous CD3 zeta intracellular domain sequence encoding SEQ ID NO: 9; and/or the CAL or CAR comprises SEQ ID NO: 15 and said germline genome comprises an endogenous CD28 intracellular domain sequence encoding SEQ ID NO: 15; and/or the CAL or CAR comprises SEQ ID NO: 18 and said germline genome comprises an endogenous 4-1BB intracellular domain sequence encoding SEQ ID NO: 18.
5. The transplant of aspect 2, 3 or 4, for treating or preventing a disease or condition (eg, a cancer) in a human patient, wherein the patient is said donor.
6. The cell or transplant of any one of aspects 1 to 5, wherein the CAL or CAR comprises an extracellular CD3 (eg, CD3 delta) or CD16 (eg, CD16A) extracellular domain. Optionally, the CD3 extracellular domain is a CD3γ, CD3δ or CDE domain.
   The invention also provides the following concepts:—
1. A chimaeric antigen ligand (CAL), wherein the CAL comprises an engineered polypeptide comprising (in N- to C-terminal direction) a CD3 extracellular domain (eg, a CD3δ or CD3E extracellular domain); an optional hinge (eg, a CD8α hinge); a transmembrane domain (eg, a CD8α or CD28 transmembrane domain); and a CD3 intracellular signalling domain; wherein when the CAL is comprised by an immune cell membrane and the CAL engages a bridging agent, intracellular signaling is triggered in the immune cell to regulate immune cell activity.
   Optionally, the transmembrane domain is is not a CD3 domain.
   In an example, the CAL comprises a human CD3δ extracellular domain. In an example, the CAL comprises a human CD8α hinge. In an example, the CAL comprises a human CD8α or CD28 transmembrane domain. In an example, the CAL comprises a human CD3 domain. The provision of a CD3 extracellular domain in an engineered polypeptide also comprising a CD3 intracellular signalling domain is a non-naturally-occurring configuration.
   Optionally, each said CD3 domain may be according to any CD3 domain described herein eg, comprising one or more CD3 domain SNPs described herein.

In an alternative, Concept 1 provides:—
A CAL polypeptide complex comprising at least first and second polypeptides, wherein
(i) the first polypeptide comprises (in N- to C-terminal direction) a CD3 extracellular domain (eg, a CD3δ or CD3E extracellular domain); an optional hinge (eg, a CD8α hinge) and a transmembrane domain (eg, a CD36, CD3E, CD8α or CD28 transmembrane domain); and
(ii) the second polypeptide comprises (in N- to C-terminal direction) a transmembrane domain (eg, a CD3, CD8α or CD28 transmembrane domain); and a CD3 intracellular signalling domain; wherein one or both or said polypeptides comprises an intracellular signalling domain that is not a CD3 domain (eg, a 4-1BB and/or CD28 intracellular signalling domain); and
wherein when the CAL is comprised by an immune cell membrane and the CAL engages a bridging agent, intracellular signaling is triggered in the immune cell to regulate immune cell activity.
In an example, the CAL comprises (a) a CD36, CD3E or CD3γ chain and (b) a CD3 chain; wherein one or both or said chains comprises an intracellular signalling domain that is not a CD3 domain (eg, a 4-1BB and/or CD28 intracellular signalling domain). For example, one or both chains each comprises one or more intracellular signalling domains each selected from the group consisting of a CD27 domain, CD28 domain, ICOS domain, OX40 domain, CD40 domain, 4-1BB domain, a FcεRlγ domain, CD64 domain and a CD16 domain, eg, comprising a CD28 intracellular signalling domain and intracellular signalling 4-1BB domain. In an example, the CAL comprises a CD3δ chain (eg, one said chain); optionally also the CAL comprises a CD3E chain (eg, 2 said chains) and/or the CAL comprises a CD3γ chain (eg, one said chain); optionally the CAL comprises a CD3 chain (eg, 2 said chains). In an example, the chain of (a) comprises a human extracellular CD3 domain (eg, a CD3δ domain). In an example, the CAL comprises a complex of one CD3δ chain, 2 CD3E chains, one CD3γ chain and two CD3 chains wherein at least one of said chains (eg, both CD3 chains) comprises a respective said intracellular signalling domain that is not a CD3 domain (eg, a 4-1BB and/or CD28 intracellular signalling domain). The CAL may, for example, comprise a naturally-occurring CD3 chain complex, comprising a complex of CD3 γ, δ, ε and chains but wherein one or more chains thereof (eg, one or more ζ chains or δ chain(s) thereof) comprises a said non-CD3 intracellular signalling domain (eg, a 4-1BB or CD28 intracellular signalling domain).
In an aspect, the invention provides an engineered CD3 chain comprising one or more intracellular signalling domains that are not CD3 domains (eg, each is a 4-1BB and/or CD28 intracellular signalling domain). For example, each non-CD3 domain is selected from the group consisting of a CD27 domain, CD28 domain, ICOS domain, OX40 domain, CD40 domain, 4-1BB domain, a FcεRlγ domain, CD64 domain and a CD16 domain, eg, comprising a CD28 intracellular signalling domain and intracellular signalling 4-1BB domain. Optionally, the CD3 chain comprises a CD3 (eg, CD3δ or CD3E) extracellular domain or a CD16 (eg, CD16A) extracellular domain. The extracellular domain may be a human domain.
In an example, all CD3 chains or domains are human, eg, derived from the same human genome (eg, the genome of a patient or prospective recipient of the CAL), eg, derived from a human donor cell or tissue (eg, bone marrow or haematopoietic stem cell sample).
In an example, the CAL of the invention is comprised by an immune cell (eg, T-, NK or TIL cell) membrane.
2. The CAL of concept 1, wherein the CD3 extracellular domain is a human CD3 extracellular domain.
3. The CAL of concept 1 or 2, comprising one or more further intracellular signalling domains, each selected from the group consisting of a CD27 domain, CD28 domain, ICOS domain, OX40 domain, CD40 domain, 4-1BB domain, a FcεRlγ domain, CD64 domain and a CD16 domain, eg, comprising a CD28 intracellular signalling domain and intracellular signalling 4-1BB domain.
   In an example, the engineered polypeptide comprises in N- to C-terminal direction said CD3 intracellular signalling domain and one or more of said further intracellular signalling domains (eg, 4-1BB and/or CD28).
   In an example, the engineered polypeptide comprises in N- to C-terminal direction one or more of said further intracellular signalling domains (eg, 4-1BB and/or CD28) and said CD3 intracellular signalling domain.
   In an example, the engineered polypeptide comprises in N- to C-terminal direction a said further intracellular signalling domain (eg, 4-1BB or CD28), said CD3 intracellular signalling domain and one or more of said further intracellular signalling domains (eg, 4-1BB and/or CD28).
4. A chimaeric antigen ligand (CAL)-immune cell (eg, a human T-, NK or TIL cell), wherein the cell expresses a CAL according to any one of concepts 1 to 3.
5. A nucleic acid encoding a CAL according to any one of concepts 1 to 3.
6. A method of producing a CAL, the method comprising expressing a CAL according to any one of concepts 1 to 3 in a cell (eg, a human T-, NK or TIL cell).
   In certain aspects of the invention, the bridging agent is a multispecific agent that is capable of binding the first target antigen (eg, a TAA expressed on a cancer cell, eg, CD19) and also capable of binding a second target antigen that is naturally surface expressed by cells of humans or a patient to which the bridging agent may be administered. In this case, for example, where the cells are immune cells (eg, T-, NK or TIL cells) the bridging agent when not bound to a CAL-immune cell of the invention, is capable to bind a wild-type (ie, endogenous) immune cell of humans or said patient. For example, the bridging agent is capable of binding a CD3 extracellular domain (eg, a CD3δ extracellular domain), wherein the bridging agent is capable of triggering intracellular signalling when bound to the extracellular domain (second target antigen) of a CAL-immune cell of the invention, or alternatively when bound to an endogenous T-cell of a patient the bridging agent is capable of triggering intracellular signalling in the endogenous T-cell. For example, the bridging agent may be a BiTE™. For example, the bridging agent is blinatumomab or catumaxomab. In these aspects of the invention, therefore the invention advantageously comprises two ways of triggering immune cell signalling in patients to which CAL-immune cells (eg, T-cells) have been administered.

### EXAMPLES

### Example 1: Analysis of Domain Variation

On the basis of information from Ensembl, analysis of naturally-occurring variation in human immune cell proteins and domains was made. This provided information to aid purposive SNP and amino acid variation matching between engineered proteins (eg, CARs or CALs) and human cells, recipient or donor humans. Benefits of such matching according to the invention are discussed in more detail above. The inventor identified the following non-synonymous SNP variation, which has utility in the present invention.

### (a) Human CD3 Variation

TABLE 1
Selected Human CD3-Gamma Variation

| | | | MINOR | AMINO | AMINO |
|---|---|---|---|---|---|
| | | NUCLEOTIDE | ALLELE | ACID | ACID |
| SNP ID | CHROMOSOME:bp | VARIATION | FREQ | VARIATION | POSITION* |
| rs3753058 | 11:118350635 | G/T | 0.298 (T) | V/F | 131 |
| rs201529449 | 11:118352416 | C/A/G | 0.014 (A) | R/G | 166 |
| rs139781104 | 11:118349035 | G/A | 0.004 (A) | A/T | 22 |
| rs143990986 | 11:118344456 | C/G | 0.001 (G) | I/M | 11 |
| rs142915569 | 11:118349821 | T/A/C | 0.001 (C) | I/N | 53 |
| rs142915569 | 11:118349821 | T/A/C | 0.001 (C) | I/T | 53 |
| rs148191859 | 11:118352431 | T/C | 0.001 (C) | Y/H | 171 |

*With reference to the product of transcript ENST00000532917

TABLE 2
Selected Human CD3-Delta Variation

| | | NUCLE- | AMINO | |
|---|---|---|---|---|
| | | OTIDE | ACID | AMINO |
| | CHROMO- | VARI- | VARI- | ACID |
| SNP ID | SOME:bp | ATION | ATION | POSITION* |
| rs193284900 | 11:118340535 | A/T | N/K | 38 |
| rs141902449 | 11:118342556 | G/T | Q/K | 18 |

*With reference to the product of transcript ENST00000300692

TABLE 3
Selected Human CD3-Epsilon Variation

| | | | MINOR | AMINO | AMINO |
|---|---|---|---|---|---|
| | | NUCLEOTIDE | ALLELE | ACID | ACID |
| SNP ID | CHROMOSOME:bp | VARIATION | FREQ | VARIATION | POSITION* |
| rs35299792 | 11:118312837 | C/A/T | 0.006 (T) | A/E | 108 |
| rs35299792 | 11:118312837 | C/A/T | 0.006 (T) | A/V | 108 |
| rs140639753 | 11:118313824 | C/T | 0.002 (T) | A/V | 157 |
| rs148647954 | 11:118312725 | G/A/C/T | 0.001 (C) | D/N | 71 |
| rs148647954 | 11:118312725 | G/A/C/T | 0.001 (C) | D/H | 71 |
| rs148647954 | 11:118312725 | G/A/C/T | 0.001 (C) | D/Y | 71 |

*With reference to the product of transcript ENST00000361763

TABLE 4
Selected Human CD3 Zeta Variation
CD3-zeta: Uniprot P20963

| | | NUCLE- | AMINO | |
|---|---|---|---|---|
| | | OTIDE | ACID | AMINO |
| | CHROMO- | VARI- | VARI- | ACID |
| SNP ID | SOME:bp | ATION | ATION | POSITION |
| s368651001 | 1:167440671 | C/T | R/K | 52 |
| rs372651048 | 1:167439396 | C/A | S/I | 56 |
| rs767112686 | 1:167439388 | C/T | A/T | 59 |
| rs765877580 | 1:167439381 | G/A | A/V | 61 |
| rs751145956 | 1:167439382 | C/G | A/P | 61 |
| rs772867144 | 1:167439379 | G/A/C | P/S | 62 |
| rs772867144 | 1:167439379 | G/A/C | P/A | 62 |
| rs55893506 | 1:167439376 | C/G | A/P | 63 |
| rs761710510 | 1:167439365 | C/G | Q/H | 66 |
| rs776601547 | 1:167439364 | C/T | G/S | 67 |
| rs768607376 | 1:167439357 | T/G | N/T | 69 |
| rs193922741 | 1:167439353- | CTG/ATA | Q/Y | 70 |
| | 167439355 | | | |
| rs193922740 | 1:167439354 | T/A/G | Q/L | 70 |
| rs193922740 | 1:167439354 | T/A/G | Q/P | 70 |
| rs193922739 | 1:167439354- | TG/CA | Q/W | 70 |
| | 167439355 | | | |
| rs780188126 | 1:167439348 | T/C | Y/C | 72 |
| rs772128174 | 1:167439346 | T/A | N/Y | 73 |
| rs757978223 | 1:167438634 | C/A | R/L | 79 |
| rs779397562 | 1:167438635 | G/C | R/G | 79 |
| rs749926653 | 1:167438626 | C/T | E/K | 82 |
| rs181746205 | 1:167438619 | T/C/G | D/G | 84 |
| rs181746205 | 1:167438619 | T/C/G | D/A | 84 |
| rs753572867 | 1:167438620 | C/A | D/Y | 84 |
| rs371709798 | 1:167438617 | C/T | V/I | 85 |
| rs145407267 | 1:167438610 | T/C | D/G | 87 |
| rs143180729 | 1:167438607 | T/C | K/R | 88 |
| rs148513413 | 1:167438602 | G/A | R/C | 90 |
| rs367690333 | 1:167438595 | C/T | R/Q | 92 |
| rs144963570 | 1:167438596 | G/A | R/W | 92 |
| rs770320255 | 1:167438593 | C/G/T | D/H | 93 |
| rs770320255 | 1:167438593 | C/G/T | D/N | 93 |
| rs139926301 | 1:167438582 | C/A | M/I | 96 |
| rs56297636 | 1:167438581 | C/T | G/R | 97 |
| rs760895755 | 1:167438571 | G/A | P/L | 100 |
| rs112890541 | 1:167435433 | T/A/G | Q/L | 101 |
| rs112890541 | 1:167435433 | T/A/G | Q/P | 101 |
| rs370910340 | 1:167435427 | C/T | R/K | 103 |
| rs145505909 | 1:167435425 | T/C | K/E | 104 |
| rs754935006 | 1:167435420 | G/T | N/K | 105 |
| rs751583971 | 1:167435418 | G/C | P/R | 106 |
| rs766541481 | 1:167435412 | T/G | E/A | 108 |
| rs763074967 | 1:167435406 | A/T | L/Q | 110 |
| rs745871212 | 1:167434054 | G/A | A/V | 120 |
| rs372665461 | 1:167434048 | G/A/C | A/V | 122 |
| rs372665461 | 1:167434048 | G/A/C | A/G | 122 |
| rs764185491 | 1:167434030 | A/G | M/T | 128 |
| rs756340039 | 1:167434027 | T/C | K/R | 129 |
| rs773572491 | 1:167434022 | C/T | E/K | 131 |
| rs201594815 | 1:167433058 | C/T | R/H | 132 |
| rs781510519 | 1:167433059 | G/A | R/C | 132 |
| rs147527561 | 1:167433055 | C/T | R/Q | 133 |
| rs751981677 | 1:167433056 | G/A | R/W | 133 |
| rs763532939 | 1:167433045 | C/A | K/N | 136 |
| rs753278244 | 1:167433043 | C/T | G/E | 137 |
| rs771873949 | 1:167433034 | C/T | G/D | 140 |
| rs186004179 | 1:167431743 | G/A | L/F | 145 |
| rs762773775 | 1:167431733 | G/T | A/D | 148 |
| rs748158220 | 1:167431721 | G/A | T/I | 152 |
| rs776703680 | 1:167431719 | A/T | Y/N | 153 |
| rs561262982 | 1:167431713 | C/T | A/T | 155 |
| rs758846009 | 1:167431709 | A/G | L/P | 156 |
| rs746262183 | 1:167431697 | G/A | A/V | 160 |
| rs376046446 | 1:167431688 | G/C | P/R | 163 |
| rs201937405 | 1:167431685 | C/T | R/H | 164 |
| rs752198795 | 1:167431686 | G/A | R/C | 164 |

Positions with reference to the following, the amino acid and nucleotide sequences of which are incorporated herein by reference.

| Name | Transcript ID | bp | Protein | UniProt |
|---|---|---|---|---|
| CD247-002 | ENST00000362089 | 1609 | 164aa | P20963 |

TABLE 5
CD3-zeta topology

| | Residue | |
|---|---|---|
| | (position) | Length |
| Feature key | Numbers* | (amino acids) |
| Signal peptide | 1-21 | 21 |
| Extracellular domain | 22-30 | 9 |
| Transmembrane | 31-51 | 21 |
| Intracellular domain | 52-164 | 113 |

*with reference to Ensembl transcript ID ENST00000392122 and protein identified by Uniprot number P20963, the sequences of which are incorporated herein by reference in their entirety.

The following positions are phosphorylated following T-cell receptor triggering: S58, Y64, Y72, Y83, Y111, Y123, Y142 and Y153. Bold=ITAM residues: in a preferred embodiment of the invention the CD3-zeta signalling domain of the engineered protein, CAR or CAL comprises Y72 and Y152 at least, and preferably all 6 tyrosines.

ITAMs (each 29 amino acids)
Positions 61-89 (positions with reference to SEQ
ID NO: 7)
APAYQQGQNQLYNELNLGRREEYDVLDKR
Positions 100-128
PQRRKNPQEGLYNELQKDKMAEAYSEIGM
Positions 131-159
ERRRGKGHDGLYQGLSTATKDTYDALHMQ

With reference to position numbers in protein product of Ensembl transcript number ENST00000392122:—

The inventor identified the following variant positions for potential matching of one or more of these according to the invention (numbering according to positions in SEQ ID NO: 7).

R52, S56, A59, A61, P62, A63, Q66, G67, N69, Q70, Y72, N73, R79, E82, D84, V85, D87, K88, R90, R92, D93, M96, G97, P100, R102, 8103, 1104, N105, E107, L109, A119, A121, M127, K128, E130, R131, R132, K135, G136, G139, L144, A147, T151, Y152, A154, L155, A159, P162, and R163.

On this basis, the inventor identified a universal human CD3-zeta intracellular domain framework, wherein certain positions are constant for universal compatibility with most human patients and human cells and ITAM tyrosines are retained for use in intracellular signaling cascades. In this respect, see SEQ ID NO: 9, which shows the universal CD3-zeta framework intracellular domain of the invention (wherein X=any amino acid). The invention, thus provides, engineered protein, a CAR or a CAL comprising an intracellular CD3-zeta domain comprising SEQ ID NO: 9. In an example, the engineered protein, CAR or CAL is expressed by an immune cell, eg a T-cell, NK cell or TIL. In an example, the invention provides a method of treating or reducing the risk of a disease or condition (eg, as disclosed herein) in a human, the method comprising administering the immune cell (eg, CAR-cell or CAL-cell) to the human, wherein the human comprises a CD3-zeta intracellular domain nucleotide sequence that encodes SEQ ID NO: 9. Thus, the protein (eg, CAR or CAL) is matched for compatibility with the patient.

### (b) Human CD28 Variation

CD28 (Cluster of Differentiation 28; Uniprot P10747) is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular). CD28 is the receptor for CD80 (B7.1) and CD86 (B7.2) proteins.

### Topology:

transmembrane=positions 153-179;
intracellular domain=positions 180-220
(position numbering with reference to SEQ ID NO: 13).

CD28 possesses an intracellular domain with several residues that are critical for its effective signaling. The YMNM motif beginning at tyrosine 170 in particular is critical for the recruitment of SH2-domain containing proteins, especially PI3K, Grb2 and Gads. The Y170 residue is important for the induction of Bcl-xL via mTOR and enhancement of IL-2 transcription via PKCθ, but has no effect on proliferation and results a slight reduction in IL-2 production. The N172 residue (as part of the YMNM) is important for the binding of Grb2 and Gads and seems to be able to induce IL-2 mRNA stability but not NE-κB translocation. The induction of NE-κB seems to be much more dependent on the binding of Gads to both the YMNM and the two proline-rich motifs within the molecule. However, mutation of the final amino acid of the motif, M173, which is unable to bind PI3K but is able to bind Grb2 and Gads, gives little NE-κB or IL-2, suggesting that those Grb2 and Gads are unable to compensate for the loss of PI3K. IL-2 transcription appears to have two stages; a Y170-dependent, PI3K-dependent initial phase which allows transcription and a PI3K-independent second phase which is dependent on formation of an immune synapse, which results in enhancement of IL-2 mRNA stability. Both are required for full production of IL-2.

CD28 also contains two proline-rich motifs that are able to bind SH3-containing proteins. Itk and Tec are able to bind to the N-terminal of these two motifs which immediately succeeds the Y170 YMNM; Lck binds the C-terminal. Both Itk and Lck are able to phosphorylate the tyrosine residues which then allow binding of SH2 containing proteins to CD28. Binding of Tec to CD28 enhances IL-2 production, dependent on binding of its SH3 and PH domains to CD28 and PIP3 respectively. The C-terminal proline-rich motif in CD28 is important for bringing Lck and lipid rafts into the immune synapse via filamin-A. Mutation of the two prolines within the C-terminal motif results in reduced proliferation and IL-2 production but normal induction of Bcl-xL. Phosphorylation of a tyrosine within the PYAP motif (Y191 in the mature human CD28) forms a high affinity-binding site for the SH2 domain of the src kinase Lck which in turn binds to the serine kinase PKC-θ.

On this basis and natural SNP non-synonymous variation analysis, the inventor identified a universal human CD28 intracellular domain framework, wherein certain positions are constant for universal compatibility with most human patients and human cells and conserved YMNM and PYAP tyrosines are retained for use in intracellular signaling cascades. In this respect, see SEQ ID NO: 15, which shows the universal CD28 framework intracellular domain of the invention (wherein X=any amino acid). The invention, thus provides an engineered protein (eg, a CAR or a CAL) comprising an intracellular CD28 domain comprising SEQ ID NO:15. In an example, the protein (eg, CAR or CAL) is expressed by an immune cell, eg a T-cell, NK cell or TIL. In an example, the invention provides a method of treating or reducing the risk of a disease or condition (eg, as disclosed herein) in a human, the method comprising administering the immune cell (eg, CAR-cell or CAL-cell) to the human, wherein the human comprises a CD28 intracellular domain nucleotide sequence that encodes SEQ ID NO: 15. Thus, the protein, CAR or CAL is matched for compatibility with the patient.

TABLE 6
Selected Human CD28 Variation
TRANSMEMBRANE VARIATION

| | | NUCLE- | AMINO | |
|---|---|---|---|---|
| | | OTIDE | ACID | AMINO |
| | CHROMO- | VARI- | VARI- | ACID |
| SNP ID | SOME:bp | ATION | ATION | POSITION |
| rs202063928 | 2:203729702 | T/C | V/A | 155 |
| rs766949965 | 2:203729707 | G/A | V/M | 157 |
| rs754393901 | 2:203729710 | G/C | V/L | 158 |
| rs760037585 | 2:203729734 | T/G | Y/D | 166 |
| rs765629224 | 2:203729737 | A/G | S/G | 167 |
| rs751287036 | 2:203729742 | G/T | L/F | 168 |
| rs201163391 | 2:203729747 | T/C | V/A | 170 |
| rs780686361 | 2:203729756 | C/T | A/V | 173 |
| rs749985173 | 2:203729758 | T/G | F/V | 174 |
| rs202069447 | 2:203729761 | A/G | I/V | 175 |
| rs755572170 | 2:203729764 | A/T | I/F | 176 |
| rs780022417 | 2:203729768 | T/A | F/Y | 177 |
| rs763432798 | 2:203734784 | G/A | V/M | 179 |

INTRACELLAR DOMAIN VARIATION

| | | NUCLE- | AMINO | |
|---|---|---|---|---|
| | | OTIDE | ACID | AMINO |
| | CHROMO- | VARI- | VARI- | ACID |
| SNP ID | SOME:bp | ATION | ATION | POSITION |
| rs139881881 | 2:203734809 | T/A/C | L/Q | 187 |
| rs139881881 | 2:203734809 | T/A/C | L/P | 187 |
| rs751945323 | 2:203734812 | A/C | H/P | 188 |
| rs753396357 | 2:203734814 | A/C | S/R | 189 |
| rs754453810 | 2:203734824 | T/C | M/T | 192* |
| rs200221759 | 2:203734829 | A/G | M/V | 194* |
| rs562969933 | 2:203734832 | A/T | T/S | 195 |
| rs765515314 | 2:203734839 | G/A | R/H | 197 |
| rs145761335 | 2:203734841 | C/T | R/C | 198 |
| rs199647272 | 2:203734842 | G/A | R/H | 198 |
| rs200751829 | 2:203734847 | G/A | G/R | 200 |
| rs201547332 | 2:203734848 | G/T | G/V | 200 |
| rs200642723 | 2:203734850 | C/T | P/S | 201 |
| rs367908475 | 2:203734856 | C/T | R/C | 203 |
| rs199549636 | 2:203734857 | G/A/C | R/H | 203 |
| rs199549636 | 2:203734857 | G/A/C | R/P | 203 |
| rs749688881 | 2:203734862 | C/T | H/Y | 205 |
| rs769098383 | 2:203734863 | A/G | H/R | 205 |
| rs572738990 | 2:203734866 | A/G | Y/C | 206 |
| rs200606770 | 2:203734871 | C/T | P/S | 208** |
| rs371850110 | 2:203734874 | T/C | Y/H | 209** |
| rs201773411 | 2:203734878 | C/G | A/G | 210** |
| rs762144222 | 2:203734881 | C/T | P/L | 211** |
| rs770610915 | 2:203734883 | C/G | P/A | 212 |
| rs199777674 | 2:203734886 | C/T | R/C | 213 |
| rs201909740 | 2:203734889 | G/A | D/N | 214 |
| rs200016310 | 2:203734895 | G/A | A/T | 216 |
| rs200936737 | 2:203734904 | C/T | R/C | 219 |
| rs201598596 | 2:203734905 | G/A | R/H | 219 |
| rs762747357 | 2:203734908 | C/T | S/F | 220 |

*part of the YMNM motif
**part of the PYAP motif

### (c) Human 4-1BB Variation

CD137 is a member of the tumor necrosis factor (TNF) receptor family. Its alternative names are tumor necrosis factor receptor superfamily member 9 (TNFRSF9), 4-1BB and induced by lymphocyte activation (ILA). It is currently of interest to immunologists as a co-stimulatory immune checkpoint molecule. The best characterized activity of CD137 is its costimulatory activity for activated T cells. Crosslinking of CD137 enhances T cell proliferation, IL-2 secretion survival and cytolytic activity. Further, it can enhance immune activity to eliminate tumors in mice.

Ensembl transcript: ENST00000615230

### Topology: Transmembrane: 187-213

Intracellular domain: 214-255
(position numbers with respect to SEQ ID NO: 16)

TABLE 7
Selected Human 4-1BB (CD137) Variation
INTRACELLULAR DOMAIN VARIATION

| | | NUCLE- | AMINO | |
|---|---|---|---|---|
| | | OTIDE | ACID | AMINO |
| | CHROMO- | VARI- | VARI- | ACID |
| SNP ID | SOME:bp | ATION | ATION | POSITION |
| rs753016242 | 1:7933197 | C/T | R/Q | 215 |
| rs143524950 | 1:7933198 | G/A | R/W | 215 |
| rs780812476 | 1:7933192 | T/C | R/G | 217 |
| rs755927735 | 1:7933187 | C/G | K/N | 218 |
| rs144908104 | 1:7933176 | T/C | Y/C | 222 |
| rs533883433 | 1:7933162 | G/A | P/S | 227 |
| rs367584804 | 1:7920916 | C/T | M/I | 229 |
| rs141498457 | 1:7920908 | A/G | V/A | 232 |
| rs751542955 | 1:7920895 | T/G | Q/H | 236* |
| rs764017912 | 1:7920887 | T/A | D/V | 239* |
| rs752191416 | 1:7920881 | C/T | C/Y | 241 |
| rs554909019 | 1:7920872 | C/T | R/Q | 244 |
| rs759184548 | 1:7920863 | T/C | E/G | 247* |
| rs776878260 | 1:7920854 | T/C | E/G | 250* |
| rs113310001 | 1:7920848 | C/A/T | G/V | 252 |
| rs113310001 | 1:7920848 | C/A/T | G/E | 252 |
| rs761088691 | 1:7920849 | C/T | G/R | 252 |
| rs772691718 | 1:7920846 | A/T | C/S | 253 |

*TRAF2 binding sites

Reference is made to: Mol Cells. 2001 Dec. 31; 12(3):304-12; “A novel leucine-rich repeat protein (LRR-1): potential involvement in 4-1BB-mediated signal transduction”; Jang L K et al, which explains that 4-1BB, a member of the tumor necrosis factor receptor (TNFR) superfamily, is induced on CD4+ and CD8+ T cells upon engagement of the T cell receptor (TCR)/CD3 complex with the antigen bound to MHC. 4-1BB plays an important role in transmitting costimulatory signal during T cell activation. However, 4-1BB-mediatded signal transduction pathways were studied. The authors conducted the yeast two-hybrid screening to identify intracellular signaling molecules that associate with 4-1BB. A novel leucine-rich repeat (LRR)-containing protein, named LRR-1, was found to specifically interact with the cytoplasmic domain of 4-1BB. Overexpression of LRR-1 suppressed the activation of NF-KB induced by 4-1BB or TNF receptor-associated factor (TRAF) 2. In addition, LRR-1 down-regulated JNK1 activity was induced by 4-1BB. The authors conclude that these results indicate that LRR-1 negatively regulates the 4-1BB-mediated signaling cascades which result in the activation of NF-kappaB and JNK1.

Reference is also made to: Mol Cell Biol. 1998 January; 18(1): 558-565; “4-1BB and Ox40 Are Members of a Tumor Necrosis Factor (TNF)-Nerve Growth Factor Receptor Subfamily That Bind TNF Receptor-Associated Factors and Activate Nuclear Factor κB”; Robert Arch & Craig Thompson, which explains that TRAF binding domains in the cytoplasmic tails of 4-1BB and OX40 are conserved between species. (A) Alignment of the protein sequences of the cytoplasmic domains of 4-1BB and Ox40. The amino acid residues shown to be important for interaction of the TRAF molecules with either receptor were studied. In this report, the authors demonstrate that 4-1BB and OX40, two members of the TNF-NGF receptor family, can use TRAF molecules to trigger cytoplasmic signal transduction cascades. This report describes the ability of the cytoplasmic domains of two members of the TNF-NGF receptor family, 4-1BB and Ox40, to bind to proteins of the TRAF family of intracellular adapter molecules. Multimerization of the cytoplasmic domains of 4-1BB and Ox40 in transfected cells can activate the transcription factor NF-κ13 in a TRAF-dependent manner. Interestingly, increased expression of individual TRAF proteins can either positively or negatively affect the ability of these receptors to induce NF-κ13 activation. These data suggest that both the differential binding affinity and relative abundance of individual TRAF proteins can influence the cellular response to receptor cross-linking. These results provide a potential explanation for the variable effects that have been observed when members of TNF-NGF receptor family are cross-linked on activated T cells.

The inventors therefore realized the desirability of conserving TRAF binding sites in the 4-1BB intracellular domain used in embodiments of engineered proteins (eg, CARs and CALs) and the desirability of matching according to the invention so that the engineered protein (eg, CAR or CAL) domain is matched for use with the intracellular machinery of the human immune cell bearing the chimaeric receptor. The invention, thus provides an engineered transmembrane protein (CAR or a CAL) or immune cell of the invention comprising this, wherein the protein comprises an intracellular 4-1BB domain comprising Q236 and E247 (eg, comprising Q236-E237-E238-D239 and 247E-248E-249E-250E wherein position numbering is with reference to SEQ ID NO: 16).

On the basis of the SNP and variation analysis, the inventor identified a universal human 4-1BB intracellular domain framework, wherein certain positions are constant for universal compatibility with most human patients and human cells and TRAF binding residues are retained for use in intracellular signaling cascades. In this respect, see SEQ ID NO: 18, which shows the universal 4-1BB framework intracellular domain of the invention (wherein X=any amino acid). The invention, thus provides, engineered protein, a CAR or a CAL comprising an intracellular 4-1BB domain comprising SEQ ID NO: 18.

In an example, the engineered protein, CAR or CAL is expressed by an immune cell, eg a T-cell, NK cell or TIL. In an example, the invention provides a method of treating or reducing the risk of a disease or condition (eg, as disclosed herein) in a human, the method comprising administering the immune cell, CAR-cell or CAL-cell to the human, wherein the human comprises a 4-1BB intracellular domain nucleotide sequence comprising Q236 and E247 (eg, comprising Q236-E237-E238-D239 and 247E-248E-249E-250E wherein position numbering is with reference to SEQ ID NO:16) or comprising SEQ ID NO: 16. Thus, the protein, CAR or CAL is matched for compatibility with the human cell or patient.

### Example 2: CD3δ-CAL T-Cell Regulation by Blinatumomab

The method disclosed in the Examples of WO2015/058018 can be readily adapted to insert a CAL-encoding transgene into immune cells (instead of a CAR-encoding transgene as disclosed in that patent application).

The CAL transgene will comprise a nucleotide sequence encoding (in N- to C-terminal direction) the human CD3δ extracellular domain of SEQ ID NO: 4 herein, a CD8α hinge and transmembrane domain, a 4-1BB intracellular domain and a CD3ζ domain. Suitably, the CAL sequence can be a modification of the CD16F-BB-ζ or CD16V-BB-ζ constructs of WO2015/058018, with the CD3δ domain sequence in place of the CD16 sequence. The transgene will be inserted into cells obtained from a human donor suffering from B-cell precursor acute lymphoblastic leukemia (ALL), and an engineered T-cell population will be developed and expanded ex vivo. The CAL and transgene will be amino acid polymorphism matched in the extracellular and intracellular CAL CD3 and 4-1BB domains. Optionally, the endogenous CD3δ and/or CD3 gene will be knocked out in the engineered cells, eg, using Cas9 targeted inactivation of the genes. Additionally the endogenous TCR loci may be rendered inactive for TCR expression.

In one trial, engineered CAL T-cells will be infused back into the donor, followed by administration of blinatumomab as bridging agent. Bridging of the CAL-cells to CD19+B-cells will lead to cancer cell killing by activated CAL T-cells in vivo, thereby treating ALL in the donor.

In another trial, the CAL T-cells will be pre-incubated with blinatumomab before infusion into the donor.

The donor will be monitored and blinatumomab titrated to regulate CAL T-cell activity.

TABLE 8
SEQUENCES

| SEQ | | |
|---|---|---|
| ID | | |
| NO: | DESCRIPTION | SEQUENCE |
| 1 | Human CD3 gamma | MEQGKGLAVLILAIILLQGTLAQSIKGNHLVKVYDYQEDGSVLLTCDAEAK |
| | sequence | NITWFKDGKMIGFLTEDKKKWNLGSNAKDPRGMYQCKGSQNKSKPLQV |
| | | YYRMCQNCIELNAATISGFLFAEIVSIFVLAVGVYFIAGQDGVRQSRASDK |
| | | QTLLPNDQLYQPLKDREDDQYSHLQGNQLRRN |
| | | I = position 53 |
| 2 | Human CD3 gamma | QSIKGNHLVKVYDYQEDGSVLLTCDAEAKNITWFKDGKMIGFLTEDKKK |
| | extracellular domain | WNLGSNAKDPRGMYQCKGSQNKSKPLQVYYRMCQNCIELNAATIS |
| | sequence | |
| 3 | Human CD3 delta | MEHSTFLSGLVLATLLSQVSPFKIPIEELEDRVFVNCNTSITWVEGTVGTLLS |
| | sequence | DITRLDLGKRILDPRGIYRCNGTDIYKDKESTVQVHYRMCQSCVELDPATV |
| | | AGIIVTDVIATLLLALGVFCFAGHETGRLSGAADTQALLRNDQVYQPLRDR |
| | | DDAQYSHLGGNWARNK |
| | | N = position 38 |
| 4 | Human CD3 delta | FKIPIEELEDRVFVNCNTSITWVEGTVGTLLSDITRLDLGKRILDPRGIYRCN |
| | extracellular domain | GTDIYKDKESTVQVHYRMCQSCVELDPATVA |
| | sequence | |
| 5 | Human CD3 epsilon | MQSGTHWRVLGLCLLSVGVWGQDGNEEMGGITQTPYKVSISGTTVILTC |
| | sequence | PQYPGSEILWQHNDKNIGGDEDDKNIGSDEDHLSLKEFSELEQSGYYVCY |
| | | PRGSKPEDANFYLYLRARVCENCMEMDVMSVATIVIVDICITGGLLLLVYY |
| | | WSKNRKAKAKPVTRGAGAGGRQRGQNKERPPPVPNPDYEPIRKGQRDL |
| | | YSGLNQRRI |
| | | A = position 108 |
| 6 | Human CD3 epsilon | DGNEEMGGITQTPYKVSISGTTVILTCPQYPGSEILWQHNDKNIGGDEDD |
| | extracellular domain | KNIGSDEDHL |
| | sequence | SLKEFSELEQSGYYVCYPRGSKPEDANFYLYLRARVCENCMEMD |
| 7 | Human CD3 zeta | MKWKALFTAAILQAQLPITEAQSFGLLDPKLCYLLDGILFIYGVILTALFLRV |
| | sequence | KFSRSADAPAYQQGQNQLYNELNLGRREEYDVLDKRRGRDPEMGGKPQ |
| | | RRKNPQEGLYNELQKDKMAEAYSEIGMKGERRRGKGHDGLYQGLSTATK |
| | | DTYDALHMQALPPR |
| 8 | Human CD3 zeta | RVKFSRSADAPAYQQGQNQLYNELNLGRREEYDVLDKRRGRDPEMGGK |
| | intracellular domain | PQRRKNPQEGLYNELQKDKMAEAYSEIGMKGERRRGKGHDGLYQGLST |
| | sequence | ATKDTYDALHMQALPPR |
| 9 | Human CD3 zeta | XVKFXRSXDXXXYQXXQXXLYXELNLGXREXYXXLXXRXGXXPEXX |
| | intracellular domain | GKXXRXXXXQXGXYNELQKDKMXEXYSEIGXXGXXXRGXXHDXLYQ |
| | sequence Universal | GXSTXTKDXYDXXHMQXLPXX |
| | Framework | Wherein X = any amino acid |
| 10 | Human CD3 zeta | APAYQQGQNQLYNELNLGRREEYDVLDKR |
| | intracellular domain | |
| | ITAM | |
| 11 | Human CD3 zeta | PQRRKNPQEGLYNELQKDKMAEAYSEIGM |
| | intracellular domain | |
| | ITAM | |
| 12 | Human CD3 zeta | ERRRGKGHDGLYQGLSTATKDTYDALHMQ |
| | intracellular domain | |
| | ITAM | |
| 13 | Human CD28 | MLRLLLALNLFPSIQVTGNKILVKQSPMLVAYDNAVNLSCKYSYNLFSREFR |
| | | ASLHKGLDSAVEVCVVYGNYSQQLQVYSKTGFNCDGKLGNESVTFYLQNL |
| | | YVNQTDIYFCKIEVMYPPPYLDNEKSNGTIIHVKGKHLCPSPLFPGPSKPFW |
| | | VLVVVGGVLACYSLLVTVAFIIFWVR |
| | | SKRSRLLHSDYMNMTPRRPGPTRKHYQPYAPPRDFAAYRS |
| | | Intracellular domain in bold |
| | | YMNM motif and PYAP motifs underlined |
| | | (first underlined Y = Y170 which is comprised by the YMNM motif) |
| | | (second underlined Y = Y191 which is comprised by the PYAP motif) |
| 14 | Human CD28 | RSKRSRLLHSDYMNMTPRRPGPTRKHYQPYAPPRDFAAYRS |
| | intracellular domain | |
| 15 | Human CD28 | RSKRSRLXXXDYMNMTPXXPXXTXKXXQPYAPXXXFXAYXX |
| | intracellular domain | Where X = any amino acid |
| | Universal Framework | |
| 16 | Human 4-1BB | MGNSCYNIVATLLLVLNFERTRSLQDPCSNCPAGTFCDNNRNQICSPCPP |
| | | NSFSSAGGQRTCDICRQCKGVFRTRKECSSTSNAECDCTPGFHCLGAGCS |
| | | MCEQDCKQGQELTKKGCKDCCFGTFNDQKRGICRPWTNCSLDGKSVLV |
| | | NGTKERDVVCGPSPADLSPGASSVTPPAPAREPGHSPQIISFFLALTSTALL |
| | | FLLFFLTLRFSVVKRGRKKLLYIFKQPFMRPVQTTQEEDGCSCRFPEEEEG |
| | | GCEL |
| | | Intracellular domain bold |
| | | (TRAF binding sites in double underlined (Q236-E237-E238-D239 |
| | | and 247E-248E-249E-250E)) |
| 17 | Human 4-1BB | KRGRKKLLYIFKQPFMRPVQTTQEEDGCSCRFPEEEEGGCEL |
| | intracellular domain | |
| 18 | Human 4-1BB | KXGXXKLLXIFKQXFXRPXQTTQEEDGXSCXFPEEEEGXXEL |
| | intracellular domain | |
| | Universal Framework | Where X = any amino acid |
| 19 | blinatumomab | DIQLTQSPASLAVSLGQRATISCKASQSVDYDGDSYLNWYQQIPGQPPKLL |
| | | IYDASNLVSGIPPRFSGSGSGTDFTLNIHPVEKVDAATYHCQQSTEDPWTF |
| | | GGGTKLEIKGGGGSGGGGSGGGGSQVQLQQSGAELVRPGSSVKISCKAS |
| | | GYAFSSYWMNWVKQRPGQGLEWIGQIWPGDGDTNYNGKFKGKATLT |
| | | ADESSSTAYMQLSSLASEDSAVYFCARRETTTVGRYYYAMDYWGQGTTV |
| | | TVSSGGGGSDIKLQQSGAELARPGASVKMSCKTSGYTFTRYTMHWVKQR |
| | | PGQGLEWIGYINPSRGYTNYNQKFKDKATLTTDKSSSTAYMQLSSLTSEDS |
| | | AVYYCARYYDDHYCLDYWGQGTTLTVSSVEGGSGGSGGSGGSGGVDDI |
| | | QLTQSPAIMSASPGEKVTMTCRASSSVSYMNWYQQKSGTSPKRWIYDTS |
| | | KVASGVPYRFSGSGSGTSYSLTISSMEAEDAATYYCQQWSSNPLTFGAGT |
| | | KLELKHHHHHH |

1. A method of bridging an immune cell and a target cell, the method comprising combining:
a) a bridging agent having a molecular weight of less than 125 kDa and comprising:
i. a first binding moiety comprising a first antibody fragment; and
ii. a second binding moiety comprising a second antibody fragment;
b) an immune cell expressing a transmembrane protein comprising an engineered combination of:
iii. an extracellular part comprising a first antigen that is linked to a transmembrane domain; wherein the second binding moiety and the first antigen form a first specific binding pair wherein the second binding moiety specifically binds to the first antigen; and
iv. an intracellular part comprising a first signalling domain for intracellular signalling when the second binding moiety and first antigen bind together; and
c) a target cell comprising a second antigen, wherein the second antigen is extracellular, and wherein
v. the first binding moiety and the second antigens form a second specific binding pair
wherein the first binding moiety specifically binds to the second antigen;
whereby the bridging agent bridges the immune cell and the target cell.

2. The method of claim 1, wherein
(a) wherein the first and second binding moieties are linked scFvs; or
(b) each binding moiety is selected from the group consisting of an scFv, nanobody, and dAb.

3. The method of claim 1, wherein the bridging agent
(a) is a bi- or tri-specific antigen binding fragment comprising two or three scFvs respectively; or
(b) is or comprises a bispecific T-cell engager antibody, bispecific-scFv, trispecific scFv, tandab, dAb, nanobody multimer, nanobody dimer, nanobody timer, dAb multimer, dAb dimer, dAb trimer, diabody, triabody, tetrabody, or Dual-affinity Re-targeting Antibody (DART).

4. The method of claim 1, wherein the first antigen is an immune cell extracellular antigen, a human T extracellular antigen, or a human NK-cell extracellular antigen.

5. The method of claim 1, wherein the bridging agent comprises antigen binding fragments of blinatumomab or a CD3/CD19-binding derivative thereof; blinatumomab or a CD3/CD19-binding derivative thereof and wherein the target cell an acute lymphoblastic leukaemia (ALL) B-cell; AMG211 or a CD3/CEA-binding derivative thereof; AMG211 or a CD3/CEA-binding derivative thereof wherein the target cell is a gastrointestinal cancer cell; pasotuxizumab or a CD3/PMSA-binding derivative thereof; pasotuxizumab or a CD3/PMSA-binding derivative thereof wherein the target cell is a prostate cancer cell; solitomab or a CD3/EpCAM-binding derivative thereof; solitomab or a CD3/EpCAM-binding derivative thereof wherein the target cell is a cancer cell; AFM11 or a CD3/CD19-binding derivative thereof; or AFM11 or a CD3/CD19-binding derivative thereof wherein the target cell is an ALL cell or Non-Hodgkin's Lymphoma cell.

6. The method of claim 1, wherein the molecular weight of the bridging agent is from 60 to 100 kDa.

7. The method of claim 1, wherein the first antigen is a protein and wherein
(a) the immune cell comprises a first nucleotide sequence that is an endogenous sequence that expresses an amino acid sequence that is identical to the amino acid sequence of the first antigen; and/or
(b) the first antigen is encoded in the cell by a non-endogenous nucleotide sequence (S1) comprising a human single nucleotide polymorphism (SNP1) that encodes an amino acid residue (R1) of the first antigen; the genome of the cell comprises a second nucleotide sequence (S2) comprising SNP1 and (i) encoding an amino acid sequence that is identical to the amino acid sequence of the first antigen and comprises R1; or (ii) encoding an amino acid sequence that is a naturally-occurring variant of the amino acid sequence of the first antigen and comprises R1; and wherein S2 is an endogenous genomic sequence of the cell and SNP1 is a non-synonymous SNP.

8. The method of claim 1, wherein the first antigen is a human protein, the subject is a human and
the first antigen is encoded in the immune cell by a non-endogenous nucleotide sequence (S1) comprising a human single nucleotide polymorphism (SNP1) that encodes an amino acid residue (R1) of the first antigen.

9. The method of claim 1, wherein the immune cell is a Tumour-Infiltrating Lymphocyte (TIL), natural killer (NK) cell or T-cell.

10. The method of claim 1, wherein
A. the first signalling domain is a human CD3 intracellular domain selected from a CD3 (CD3-zeta) domain and a CD3η (CD3-eta) domain, and comprises (a) Y72, Y83, Y111, Y123, Y142 and Y153 (position numbers correspond to positions of SEQ ID NO: 7) or (b) one, two or three amino acid motifs selected from (i) SEQ ID NO: 10; (ii) SEQ ID NO: 11; and (iii) SEQ ID NO: 12;
B. the first signalling domain is a human CD28 intracellular domain (c) comprising at least 13 amino acid residues selected from the group consisting of R180, 5181, K182, R183, 5184, R185, L186, D190, Y191, N193, P196, P199, T202, K204, Q207, F215, A217 and Y218 (position numbers correspond to positions of SEQ ID NO: 13), or
C. the first signalling domain is a human CD28 intracellular domain (c) comprising at least 13 amino acid residues selected from the group consisting of R180, 5181, K182, R183, 5184, R185, L186, D190, Y191, N193, P196, P199, T202, K204, Q207, F215, A217 and Y218 (position numbers correspond to positions of SEQ ID NO: 13), and wherein the CD28 domain comprises (d) a YMNM motif (corresponding to Y191-M192-N193-M194 of SEQ ID NO: 13) and/or (e) a PYAP motif (corresponding to P208-Y209-A210-P211 of SEQ ID NO: 13).

11. The method of claim 11, wherein the target cell genome comprises:
D. an endogenous nucleotide sequence encoding a human CD3-zeta or CD3-eta intracellular domain comprising Y72, Y83, Y111, Y123, Y142 and Y153 (position numbers correspond to positions of SEQ ID NO: 7), when the first signalling domain comprises motif (a) according to claim 10A;
E. an endogenous nucleotide sequence encoding a human CD3-zeta or CD3-eta intracellular domain comprising SEQ ID NO: 10, when the first signalling domain comprises motif (b(i)) according to claim 10A;
F. an endogenous nucleotide sequence encoding a human CD3-zeta or CD3-eta intracellular domain comprising SEQ ID NO: 11, when the first signalling domain comprises motif (b)(ii) according to claim 10A;
G. an endogenous nucleotide sequence encoding a human CD3-zeta or CD3-eta intracellular domain comprising SEQ ID NO: 12, when the first signalling domain comprises motif (b)(iii) according to claim 10A;
H. an endogenous nucleotide sequence encoding a human CD28 intracellular domain comprising at least 13 amino acid residues selected from the group consisting of R180, 5181, K182, R183, S184, R185, L186, D190, Y191, N193, P196, P199, T202, K204, Q207, F215, A217 and Y218 (position numbers correspond to positions of SEQ ID NO: 13), when the first signalling domain is according to claim 10B(c);
I. an endogenous nucleotide sequence encoding a human CD28 intracellular domain comprising a YMNM motif (corresponding to Y191-M192-N193-M194 of SEQ ID NO: 13), when the first signalling domain is according to claim 10C(d): or
J. an endogenous nucleotide sequence encoding a human CD28 intracellular domain comprising a PYAP motif (corresponding to P208-Y209-A210-P211 of SEQ ID NO: 13), when the first signalling domain is according to claim 10C(e).

12. The method of claim 1,
wherein the genome of the immune cell comprises one, more, or all of A to C:
A. an endogenous nucleotide sequence encoding a CD3 zeta intracellular domain comprising SEQ ID NO: 9 or wherein the endogenous sequence has been inactivated or knocked-out; and a nucleotide sequence encoding a CD3 zeta intracellular domain of the transmembrane protein which comprises SEQ ID NO: 9;
B. an endogenous nucleotide sequence encoding a CD28 intracellular domain comprising SEQ ID NO: 15 or wherein the endogenous sequence has been inactivated or knocked-out; and a nucleotide sequence encoding a CD28 intracellular domain of the transmembrane protein which comprises SEQ ID NO: 15;
C. an endogenous nucleotide sequence encoding a 4-1BB intracellular domain comprising SEQ ID NO: 18 or wherein the endogenous sequence has been inactivated or knocked-out; and a nucleotide sequence encoding a 4-1BB intracellular domain of the transmembrane protein which comprises SEQ ID NO: 18.

13. The method of claim 12, wherein the method is carried out in a human and the germline genome of the human comprises one, more, or all of D to F:
D. an endogenous nucleotide sequence encoding a CD3 zeta intracellular domain comprising SEQ ID NO: 9; when the immune cell is according to claim 12A;
E. an endogenous nucleotide sequence encoding a CD28 intracellular domain comprising SEQ ID NO: 15; when the immune cell is according to claim 12B;
F. an endogenous nucleotide sequence encoding a 4-1BB intracellular domain comprising SEQ ID NO: 18; when the immune cell is according to claim 12C.

14. The method of claim 1, wherein
(a) the first antigen is selected from the group consisting of:
a CD3 extracellular domain, a CD16 extracellular domain, human IL-1A, human IL-1β, human IL-1RN, human IL-6, human BLys, human APRIL, human activin A, human TNF alpha, a human BMP, human BMP2, human BMP7, human BMP9, human BMP10, human GDF8, human GDF11, human RANKL, human TRAIL, human VEGFA, human VEGFB or human PGF;
(b) the first antigen comprises an extracellular CD3 domain and the second binding moiety comprises the anti-CD3 variable domains of an antibody selected from the group consisting of blinatumomab, pasotuximab, AFM11, solitumab, L2K-07, muromonab-CD3, otelixizumab, teplizumab, visilizumab, catumaxomab, ertumaxomab and foralumab; or
(c) the second antigen and the first binding moiety are selected from the group consisting of:
(i) the second antigen comprises an extracellular CD19 domain and the first binding moiety comprises an antigen binding fragment therefor, or wherein the first binding moiety comprises an antigen binding fragment comprising the anti-CD19 variable domains of an antibody selected from the group consisting of blinatumomab, HD37, and AFM11;
(ii) the second antigen comprises an extracellular EpCAM domain and the first binding moiety comprises an antigen binding fragment therefor, or wherein the first binding moiety comprises an antigen binding fragment comprising the anti-EpCAM variable domains of an antibody selected from the group consisting of catumaxomab or solitomab;
(iii) the second antigen comprises an extracellular PSMA domain and the first binding moiety comprises an antigen binding fragment therefor, or wherein the first binding moiety comprises an antigen binding fragment comprising the anti-PMSA variable domains of pasotuxizumab;
(iv) the second antigen comprises an extracellular Her1 domain and the first binding moiety comprises an antigen binding fragment therefor, or wherein the first binding moiety comprises an antigen binding fragment comprising the anti-Her1 variable domains of an antibody selected from the group consisting of RG7597, panitumumab, and LY3164530;
(v) the second antigen comprises an extracellular Her2 domain and the first binding moiety comprises an antigen binding fragment therefor, or wherein the first binding moiety comprises an antigen binding fragment comprising the anti-Her2 variable domains of an antibody selected from the group consisting of ertumaxomab, pertuzumab, trastuzumab, and MM111;
(vi) the second antigen comprises an extracellular Her3 domain and the first binding moiety comprises an antigen binding fragment therefor, or wherein the first binding moiety comprises an antigen binding fragment comprising the anti-Her3 variable domains of an antibody selected from the group consisting of RG7597, MM141, and MM111;
(vii) the second antigen comprises an extracellular CD20 domain and the first binding moiety comprises an antigen binding fragment therefor, or wherein the first binding moiety comprises an antigen binding fragment comprising the anti-CD20 variable domains of an antibody selected from the group consisting of lymphomum, ofatumumab, obinutuzumab, ibritumomab, tositumomab, and rituximab;
(viii) the second antigen comprises an extracellular angiopoietin-2 domain and the first binding moiety comprises an antigen binding fragment therefor, or wherein the first binding moiety comprises an antigen binding fragment comprising the anti-angipoietin-2 variable domains of an antibody selected from the group consisting of RG7221 and RG7716;
(ix) the second antigen comprises an extracellular CEA domain and the first binding moiety comprises an antigen binding fragment therefor, or wherein the first binding moiety comprises an antigen binding fragment comprising the anti-CEA variable domains of an antibody selected from the group consisting of TF2, AMG211, MEDI-565, and MT111;
(x) the second antigen comprises an extracellular IFG1R domain and the first binding moiety comprises an antigen binding fragment therefor, or wherein the first binding moiety comprises an antigen binding fragment comprising the anti-IFG1R variable domains of MM141;
(xi) the second antigen comprises an extracellular CD124 domain and the first binding moiety comprises an antigen binding fragment therefor, or wherein the first binding moiety comprises an antigen binding fragment comprising the anti-CD124 variable domains of MGD006;
(xii) the fourth binding moiety comprises an extracellular gpa33 domain and the first binding moiety comprises an antigen binding fragment therefor, or wherein the first binding moiety comprises an antigen binding fragment comprising the anti-gpa33 variable domains of MDF007;
(xiii) the second antigen comprises an extracellular CD30 domain and the first binding moiety comprises an antigen binding fragment therefor, or wherein the first binding moiety comprises an antigen binding fragment comprising the anti-CD30 variable domains of an antibody selected from the group consisting of AFM13 and brentuximab;
(xiv) the second antigen comprises an extracellular cMet domain and the first binding moiety comprises an antigen binding fragment therefor, or wherein the first binding moiety comprises an antigen binding fragment comprising the anti-cMet variable domains of LY3164530;
(xv) the second antigen comprises an extracellular glycoprotein IIb/IIIa domain and the first binding moiety comprises an antigen binding fragment therefor, or wherein the first binding moiety comprises an antigen binding fragment comprising the anti-glycoprotein IIb/IIIa variable domains of abciximab;
(xvi) the second antigen comprises an extracellular CD25 domain and the first binding moiety comprises an antigen binding fragment therefor, or wherein the first binding moiety comprises an antigen binding fragment comprising the anti-CD125 variable domains of an antibody selected from the group consisting of basiliximab and daclizumab;
(xvii) the second antigen comprises an extracellular respiratory syncytial virus F protein domain and the first binding moiety comprises an antigen binding fragment therefor, or wherein the first binding moiety comprises an antigen binding fragment comprising the anti-respiratory syncytial virus F protein variable domains of an antibody selected from the group consisting of palivizumab and motavizumab;
(xviii) the second antigen comprises an extracellular TNF-alpha domain and the first binding moiety comprises an antigen binding fragment therefor, or wherein the first binding moiety comprises an antigen binding fragment comprising the anti-TNF-alpha variable domains of an antibody selected from the group consisting of infliximab, certolizumab, golimumab, and adalimumab;
(xix) the second antigen comprises an extracellular CD33 domain and the first binding moiety comprises an antigen binding fragment therefor, or wherein the first binding moiety comprises an antigen binding fragment comprising the anti-CD33 variable domains of gemtuzumab;
(xx) the second antigen comprises an extracellular CD52 domain and the first binding moiety comprises an antigen binding fragment therefor, or wherein the first binding moiety comprises an antigen binding fragment comprising the anti-CD52 variable domains of alemtuzumab;
(xxi) the second antigen comprises an extracellular IgE domain and the first binding moiety comprises an antigen binding fragment therefor, or wherein the first binding moiety comprises an antigen binding fragment comprising the anti-IgE variable domains of omalizumab;
(xxii) the second antigen comprises an extracellular CD11a domain and the first binding moiety comprises an antigen binding fragment therefor, or wherein the first binding moiety comprises an antigen binding fragment comprising the anti-CD11a variable domains of efalizumab;
(xxiii) the second antigen comprises an extracellular EGFR domain and the first binding moiety comprises an antigen binding fragment therefor, or wherein the first binding moiety comprises an antigen binding fragment comprising the anti-EGFR variable domains of cetuximab;
(xxiv) the second antigen comprises an extracellular VEGF-A domain and the first binding moiety comprises an antigen binding fragment therefor, or wherein the first binding moiety comprises an antigen binding fragment comprising the anti-VEGF-A variable domains of bevacizumab and ranibizumab;
(xxv) the second antigen comprises an extracellular alpha-4 integrin domain and the first binding moiety comprises an antigen binding fragment therefor, or wherein the first binding moiety comprises an antigen binding fragment comprising the anti-alpha-4 integrin variable domains of natalizumab;
(xxvi) the second antigen comprises an extracellular IL-6R domain and the first binding moiety comprises an antigen binding fragment therefor, or wherein the first binding moiety comprises an antigen binding fragment comprising the anti-IL-6R variable domains of tocilizumab;
(xxvii) the second antigen comprises an extracellular complement protein C5 domain and the first binding moiety comprises an antigen binding fragment therefor, or wherein the first binding moiety comprises an antigen binding fragment comprising the anti-complement protein C5 domain variable domains of eculizumab;
(xxviii) the second antigen comprises an extracellular IL-1 domain and the first binding moiety comprises an antigen binding fragment therefor, or wherein the first binding moiety comprises an antigen binding fragment comprising the anti-IL-1 variable domains of canakinumab;
(xxix) the second antigen comprises an extracellular IL-12 or IL-23 domain and the first binding moiety comprises an antigen binding fragment therefor, or wherein the first binding moiety comprises an antigen binding fragment comprising the anti-IL-12 or anti-IL-23 variable domains of ustekinumab;
(xxx) the second antigen comprises an extracellular RANKL domain and the first binding moiety comprises an antigen binding fragment therefor, or wherein the first binding moiety comprises an antigen binding fragment comprising the anti-RANKL variable domains of denosumab;
(xxxi) the second antigen comprises an extracellular anthrax toxin protective antigen domain and the first binding moiety comprises an antigen binding fragment therefor, or wherein the first binding moiety comprises an antigen binding fragment comprising the anti-extracellular anthrax toxin protective antigen domain variable domains of raximbacumab;
(xxxii) the second antigen comprises an BLyS domain and the first binding moiety comprises an antigen binding fragment therefor, or wherein the first binding moiety comprises an antigen binding fragment comprising the anti-BLyS variable domains of belimumab; or
(xxxiii) the second antigen comprises an CTLA-4 domain and the first binding moiety comprises an antigen binding fragment therefor, or wherein the first binding moiety comprises an antigen binding fragment comprising the anti-CTLA-4 variable domains of ipilimumab;
(d) the second antigen comprises a polypeptide selected from the group consisting of:
a glioma-associated antigen, carcinoembryonic antigen (CEA), β-human chorionic gonadotropin, alphafetoprotein (AFP), lectin-reactive AFP, thyroglobulm, RAGE-1, MN-CA IX, human telomerase reverse transcriptase, RU1, RU2 (AS), intestinal carboxylate esterase, mut hsp70-2, M-CSF, prostase, prostate-specific antigen (PSA), PAP, NY-ESO-1, LAGE-la, p53, prostein, PSMA, survivin, telomerase, prostate-carcinoma tumour antigen-1 (PCTA-1), MAGE, ELF2M, neutrophil elastase, ephrinB2, CD22, insulin growth factor (IGF)-I, IGF-II, IGF-I receptor, mesothelin, MART-1, tyrosinase, GP 100, phosphatase (PAP), CD19, CD20, CD37, Pmel 17, TRP-1, TRP-2, MAGE-1, MAGE-3, BAGE, GAGE-1, GAGE-2, pi 5, ras, BCR-ABL, E2A-PRL, H4-RET, 1GH-IGK, MYL-RAR, Epstein Barr virus antigen (EBVA), human papillomavirus (HPV) antigen E6, HPV antigen E7, TSP-180, MAGE-4, MAGE-5, MAGE-6, RAGE, NY-ESO, pl 85 erbB2, pl 80 erbB3, c-met, nm-23H I, PSA, TAG-72, CA 19-9, CA 72-4, CAM 17.1, NuMa, K-ras, beta-Catenin, CDK4, Mum-1, p 15, p 16, 43-9F, 5T4(791Tgp72), alpha-fetoprotem, beta-HCG, BCA225, BTAA, CA 125, CA 10 15-3\CA 27.29\BCAA, CA 195, CA 242, CA-50, CAM43, CD68\ I, CO-029, FGF-5, G250, Ga733VEpCAM, HTgp-175, M344, MA-50, MG7-Ag, MOV 18, NB/70K, NY-CO-1, RCAS 1, SDCCAG16, TA-90\Mac-2 binding protein Acyclophilin C-associated protein, TAAL6, TAG72, TLP, TPS, EpCAM, MCSP, EGFR, EGFRvIII, sialyl Tn, CD133, CD33, CD30, CD47, CD52, gpA33, TAG-72, mucin, CIX, GD2, GD3, GM2, CD123, VEGFR, integrin, cMET, Her1, Her2, Her3, IGF1R, EPHA3, CD66e, EphA2, TRAILR1, TRAILR2, RANKL, FAP, Angiopoietin, tenascin, R0R1, mesothelin, CD33/IL3Ra, c-Met, Glycolipid F77, EGFRvIII, GD-2, NY-ESO-1 TCR, MAGE A3 TCR, angiopoietin-2, IFG1R, CD124,11b/11a, CD25, respiratory syncytial virus F protein, TNF-alpha, IgE, CD11a, VEGF-A, alpha-4 integrin, IL-6R, complement protein C5, IL-1, IL-12, IL-23, anthrax toxin protective antigen, BLyS, and CTLA-A;
(e) the second antigen and first binding moiety are selected from a group consisting of:
(i) the second antigen comprises human VEGFA, human VEGFB or human PGF and the first binding moiety comprises an anti-human VEGFA, human VEGVB or human PGF antigen binding fragment of aflibercept;
(ii) the second antigen comprises human TNF-alpha and the first binding moiety comprises an anti-TNF-alpha antigen binding fragment of ranibizumab, etanercept, or certolizumab;
(iii) the second antigen comprises human APRIL or human BLyS and the first binding moiety comprises an anti-BLyS antigen binding fragment of atacicept; or
(iv) the fourth binding moiety comprises human IL-1 and the first binding moiety comprises an anti-IL-1 antigen binding fragment of rilonacept,
(v) the second antigen comprises an extracellular CD3 domain and the first binding moiety comprises an antigen binding fragment comprising the anti-CD3 variable domains of an antibody selected from the group consisting of blinatumomab, pasotuximab, AFM11, solitumab, L2K-07, muromonab-CD3, otelixizumab, teplizumab, visilizumab, catumaxomab, ertumaxomab and foralumab;
(vi) the second antigen comprises an extracellular CD19 domain and the first binding moiety comprises an antigen binding fragment comprising the anti-CD19 variable domains of an antibody selected from the group consisting of blinatumomab, HD37, and AFM11;
(vii) the second antigen comprises an extracellular CEA domain and the first binding moiety comprises an anti-CEA antigen binding fragment comprising the variable domains of an antibody selected from the group consisting of TF2, AMG211, MEDI-565, and MT111;
(viii) the second antigen comprises an extracellular PSMA domain and the first binding moiety comprises an anti-PSMA antigen binding fragment comprising the variable domains of pasotuxizumab;
(ix) the second antigen comprises an extracellular EpCAM domain and the first binding moiety comprises an anti-EpCAM antigen binding fragment comprising the variable domains of an antibody selected from the group consisting of catumaxomab or solitomab.

15. The method of claim 1, wherein the bridging agent comprises a ZIP miniantibody; diabody; (scFv)2/BITE; Sc-Diabody; Barnase-barstar dimer; Minibody; (Fab)2; sc(Fab)2; scFv-Fc; Triabody; Trimerbody; Tribody; Tribi-minibody; Collabody; Barnase-barstar trimer; (scFv-TNFα)3; Tandab; [sc(Fv)2]2; Tetrabody; (scFv-p53)4; or Di-diabody.

16. The method of claim 1, wherein the first and second antibody fragments are each a ZIP scFv; Fab; dAb; or nanobody.


SEQUENCE LISTING (19 sequences)

SEQ ID NO: 1 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 2 - protein, 94 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATIS

SEQ ID NO: 3 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 4 - protein, 84 aa, Homo sapiens [sequence omitted]

SEQ ID NO: 5 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 6 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 7 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - protein, 113 aa, Homo sapiens
  misc_feature (1)..(1): Xaa can be any naturally occurring amino acid
  misc_feature (5)..(5): Xaa can be any naturally occurring amino acid
  misc_feature (8)..(8): Xaa can be any naturally occurring amino acid
  misc_feature (10)..(12): Xaa can be any naturally occurring amino acid
  misc_feature (15)..(16): Xaa can be any naturally occurring amino acid
  misc_feature (18)..(19): Xaa can be any naturally occurring amino acid
  misc_feature (22)..(22): Xaa can be any naturally occurring amino acid
  misc_feature (28)..(28): Xaa can be any naturally occurring amino acid
  misc_feature (31)..(31): Xaa can be any naturally occurring amino acid
  misc_feature (33)..(34): Xaa can be any naturally occurring amino acid
  misc_feature (36)..(37): Xaa can be any naturally occurring amino acid
  misc_feature (39)..(39): Xaa can be any naturally occurring amino acid
  misc_feature (41)..(42): Xaa can be any naturally occurring amino acid
  misc_feature (45)..(46): Xaa can be any naturally occurring amino acid
  misc_feature (49)..(50): Xaa can be any naturally occurring amino acid
  misc_feature (52)..(55): Xaa can be any naturally occurring amino acid
  misc_feature (57)..(57): Xaa can be any naturally occurring amino acid
  misc_feature (59)..(59): Xaa can be any naturally occurring amino acid
  misc_feature (69)..(69): Xaa can be any naturally occurring amino acid
  misc_feature (71)..(71): Xaa can be any naturally occurring amino acid
  misc_feature (77)..(78): Xaa can be any naturally occurring amino acid
  misc_feature (80)..(82): Xaa can be any naturally occurring amino acid
  misc_feature (85)..(86): Xaa can be any naturally occurring amino acid
  misc_feature (89)..(89): Xaa can be any naturally occurring amino acid
  misc_feature (94)..(94): Xaa can be any naturally occurring amino acid
  misc_feature (97)..(97): Xaa can be any naturally occurring amino acid
  misc_feature (101)..(101): Xaa can be any naturally occurring amino acid
  misc_feature (104)..(105): Xaa can be any naturally occurring amino acid
  misc_feature (109)..(109): Xaa can be any naturally occurring amino acid
  misc_feature (112)..(113): Xaa can be any naturally occurring amino acid
XVKFXRSXDXXXYQXXQXXLYXELNLGXREXYXXLXXRXGXXPEXXGKXXRXXXXQXGXYNELQKDKMXEXYSEIGXXGXXXRGXXHDXLYQGXSTXTKDXYDXXHMQXLPXX

SEQ ID NO: 10 - protein, 29 aa, Homo sapiens
APAYQQGQNQLYNELNLGRREEYDVLDKR

SEQ ID NO: 11 - protein, 29 aa, Homo sapiens
PQRRKNPQEGLYNELQKDKMAEAYSEIGM

SEQ ID NO: 12 - protein, 29 aa, Homo sapiens
ERRRGKGHDGLYQGLSTATKDTYDALHMQ

SEQ ID NO: 13 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 14 - protein, 41 aa, Homo sapiens
RSKRSRLLHSDYMNMTPRRPGPTRKHYQPYAPPRDFAAYRS

SEQ ID NO: 15 - protein, 41 aa, Homo sapiens
  misc_feature (8)..(10): Xaa can be any naturally occurring amino acid
  misc_feature (18)..(19): Xaa can be any naturally occurring amino acid
  misc_feature (21)..(22): Xaa can be any naturally occurring amino acid
  misc_feature (24)..(24): Xaa can be any naturally occurring amino acid
  misc_feature (26)..(27): Xaa can be any naturally occurring amino acid
  misc_feature (33)..(35): Xaa can be any naturally occurring amino acid
  misc_feature (37)..(37): Xaa can be any naturally occurring amino acid
  misc_feature (40)..(41): Xaa can be any naturally occurring amino acid
RSKRSRLXXXDYMNMTPXXPXXTXKXXQPYAPXXXFXAYXX

SEQ ID NO: 16 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 17 - protein, 42 aa, Homo sapiens
KRGRKKLLYIFKQPFMRPVQTTQEEDGCSCRFPEEEEGGCEL

SEQ ID NO: 18 - protein, 42 aa, Homo sapiens
  misc_feature (2)..(2): Xaa can be any naturally occurring amino acid
  misc_feature (4)..(5): Xaa can be any naturally occurring amino acid
  misc_feature (9)..(9): Xaa can be any naturally occurring amino acid
  misc_feature (14)..(14): Xaa can be any naturally occurring amino acid
  misc_feature (16)..(16): Xaa can be any naturally occurring amino acid
  misc_feature (19)..(19): Xaa can be any naturally occurring amino acid
  misc_feature (28)..(28): Xaa can be any naturally occurring amino acid
  misc_feature (31)..(31): Xaa can be any naturally occurring amino acid
  misc_feature (39)..(40): Xaa can be any naturally occurring amino acid
KXGXXKLLXIFKQXFXRPXQTTQEEDGXSCXFPEEEEGXXEL

SEQ ID NO: 19 - protein, 504 aa, Artificial Sequence
  BLINATUMOMAB
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH